(19) United States
(12) Patent Application Publication
(10) Pub. No.: US 2026/0083106 A1
(43) Pub. Date: Mar. 26, 2026

(54) HUMANIZED IMMUNODEFICIENT MOUSE MODELS

(71) Applicants: The Jackson Laboratory, Bar Harbor, ME (US); University of Massachusetts, Westborough, MA (US)

(72) Inventors: Leonard D. Shultz, Bar Harbor, ME (US); Michael A. Brehm, Westborough, MA (US); Dale L. Greiner, Westborough, MA (US); Charles P. Emerson, Westborough, MA (US); Katelyn DAMAN, Westborough, MA (US)

(73) Assignees: The Jackson Laboratory, Bar Harbor, ME (US); University of Massachusetts, Westborough, MA (US)

(21) Appl. No.: 19/109,249

(22) PCT Filed: Sep. 7, 2023
(86) PCT No.: PCT/US2023/073632

§ 371 Date: Mar. 6, 2025

Related U.S. Application Data
(60) Provisional application No. 63/404,597, filed on Sep. 8, 2022.

(51) Int. Cl. A01K 67/0271; A01K 67/0275; A61K 49/00; C07K 14/52; C07K 14/535; C07K 14/54

(52) U.S. Cl. CPC A01K 67/0271; A01K 67/0275; A61K 49/0008; C07K 14/52; C07K 14/535; C07K 14/5403; C07K 14/5418; C07K 14/5443; A01K 2207/12; A01K 2207/15; A01K 2217/052; A01K 2217/056; A01K 2227/105; A01K 2267/03

(57) ABSTRACT

The present disclosure provides, in some aspects, humanized immunodeficient mouse models that support long-term engraftment and function of human T cells, natural killer cells, and myeloid cells, without the need for conditioning.

### RELATED APPLICATION

This application claims the benefit under 35 U.S.C. § 119(e) of U.S. provisional application No. 63/404,597, filed on Sep. 8, 2022, which is incorporated by reference herein in its entirety.

### GOVERNMENT LICENSE RIGHTS

This invention was made with government support under AI132963 awarded by National Institutes of Health. The government has certain rights in the invention.

### REFERENCE TO AN ELECTRONIC SEQUENCE LISTING

The contents of the electronic sequence listing (J022770130W000-SEQ-EMB.xml; Size: 2,991 bytes; and Date of Creation: Sep. 5, 2023) is herein incorporated by reference in its entirety.

### BACKGROUND

Humanized mouse models are valuable preclinical tools that enable researchers to perform more translationally-relevant studies. These models are mice that contain functioning human genes, cells, tissues, or microbiota. There are three general ways to humanize a mouse: human immune system engraftment into an immunodeficient host, replacement of mouse genes with their human homologs, or transferring fecal microbiota from a human donor into a germ-free mouse.

Human immune system engraftment typically involves the engraftment of human hematopoietic stem cells (HSCs) or the engraftment of human peripheral blood mononuclear cells (PMBCs). To facilitate engraftment of the human HSCs, an immunodeficient mouse is first conditioned (e.g., irradiated or subjected to some other form of myeloablative therapy) to weaken its immune system. Additionally, the human HSCs are often manipulated to enriched for human CD34⁺ HSCs and to deplete CD3⁺ T cells. These treatments prevent or at least reduce the likelihood that the mouse will develop acute xenogeneic graft-versus-host disease (GvHD), a condition resulting in the human immune cells attacking the host mouse tissues, primarily mediated by the presence of human leukocyte antigen (HLA)-restricted T cells. For mouse models that aim to replicate human T cell and innate immunity, human PBMC engraftment is typically used; however, avoiding GvHD in these model systems has been challenging. Conditioning treatments used to suppress the mouse immune system simultaneously kill off the mature HLA-restricted T cells, so these treatments cannot be used with PBMC engraftment. Recipient mice engrafted with human PBMCs develop GvHD shortly after engraftment.

Thus, there are a number of limitations in the development of human immune systems in humanized mice that would allow their use for the study of human stem cell therapies. These limitations include difficulty in generating primary and recall human adaptive and innate cellular immune responses, including T cell priming and memory T cell generation, and low human NK cell activity. These observations exemplify a critical need for a more mature, fully functional human (adaptive and innate) immune system.

### SUMMARY

Immunodeficient mice engrafted with human immune cells are essential for evaluating the efficacy and safety of human cell therapy products that are engineered to avoid host immune rejection, for example. Typically, individual mouse models are used to evaluate specific immune biology for a specific cell type. This a la carte approach, however, results in greater overall direct mouse costs and leads to longer project timelines. Further, manipulation of the human cell samples and myeloablative conditioning of the immunodeficient mice often compromises the survival, proliferative capacity, and function of the engrafted human immune cells. Thus, new humanization methods and mouse models are needed. The present disclosure fulfills this need and advances the field by providing, in some aspects, a new humanized mouse model and humanization methods that can be used to evaluate multiple, complex aspects of human immune cell biology (e.g., human cell therapy products) in a single mouse strain, for example. The methods provided herein also aim to minimize processing of the human xenograft samples and manipulation of the immunodeficient mouse.

Thus, some aspects of the present disclosure provide a humanized immunodeficient mouse model that support long-term engraftment and function of human T cells, natural killer (NK) cells, and innate immune cells (e.g., myeloid cells such as basophils, dendritic cells, eosinophils, Langerhans cells, mast cells, monocytes and macrophages, and neutrophils), without the need for conditioning (e.g., myeloablative therapy). The humanized immunodeficient mouse models provided herein have a robust human (adaptive and innate) immune system, in part because they have little to no residual innate immunity and they transgenically express human-specific cytokines required for human T cell, NK cell and innate immune cell (e.g., myeloid cell) development and function.

In some aspects, the disclosure provides an immunodeficient non-obese diabetic (NOD) mouse comprising: an endogenous Il2rg allele comprising a null mutation; an endogenous H2-K allele comprising a null mutation; an endogenous H2-D allele comprising a null mutation; an endogenous H2-A allele comprising a null mutation; an endogenous Kit allele comprising a null mutation; an exogenous nucleic acid encoding human interleukin 7 (huIL7); an exogenous nucleic acid encoding human interleukin 15 (huIL15); an exogenous nucleic acid encoding human interleukin 3 (huIL3); an exogenous nucleic acid encoding human granulocyte-macrophage colony-stimulating factor (huGM-CSF); and an exogenous nucleic acid encoding human Steel factor (also referred to as human stem cell factor or human KIT ligand) (huSCF).

In some embodiments, the endogenous H2-K1 allele comprising a null mutation is a H2-K1^{tm1Bpe} allele. In some embodiments, the endogenous H2-D allele comprising a null mutation is a H2-D1^{tm1Bpe} allele. In some embodiments, the endogenous H2-A allele comprising a null mutation is an H2-Ab1^{em1Mvw} allele.

In some embodiments, the endogenous Interleukin 2 Receptor Gamma (Il2rg) allele comprising a null mutation is an Il2rg^{tm1Wjl} allele. In some embodiments, the endogenous Il2rg allele comprising a null mutation is an Il2rg^{tm1Sug} allele.

In some embodiments, the immunodeficient mouse further comprises an endogenous Protein Kinase, DNA-Activated, Catalytic Subunit (Prkdc) allele comprising a null mutation. In some embodiments, the mutation is a severe combined immunodeficiency (scid) mutation. In some embodiments, the endogenous Prkdc allele comprising a null mutation is a Prkdc^{scid} allele.

In some embodiments, the immunodeficient mouse further comprises an endogenous Recombination Activating Gene 1 (Rag1) allele comprising a null mutation. In some embodiments, the endogenous Rag1 allele comprising a null mutation is a Rag1^{tm1Mom} allele.

In some embodiments, the immunodeficient mouse further comprises an endogenous Recombination Activating Gene 2 (Rag2) allele comprising a null mutation. In some embodiments, the endogenous Rag2 allele comprising a null mutation is a Rag2^{tm1Fwa} allele.

In some embodiments, the immunodeficient mouse has a NOD scid gamma background. In some embodiments, the immunodeficient mouse has an NSG-(K^{b} D^{b})^{null} (IA^{null}) genetic background.

In some embodiments, the endogenous Kit allele comprising a null mutation is Kit^{W-41J}.

In some embodiments, the mouse further comprises (or has been engrafted with) human cells.

In some embodiments, the immunodeficient mouse further comprises (or has been engrafted with) unfractionated human umbilical cord blood comprising the human cells.

In some embodiments, the human cells comprise human hematopoietic stem cells. In some embodiments, the human cells comprise human peripheral blood mononuclear cells.

In some embodiments, the human cells are not enriched for CD34⁺ human hematopoietic stem cells. In some embodiments, CD3⁺ human T cells have not been depleted from the human cells.

In some embodiments, the mouse has not been subjected to myeloablation, for example, irradiation or chemical myeloablation. In other embodiments, the mouse has been subjected to myeloablation.

In other aspects, the disclosure provides a method for producing a humanized mouse, the method comprising administering human cells to any one of the immunodeficient mice described herein.

In some embodiments, the method comprises administering unfractionated human umbilical cord blood comprising the human cells. In some embodiments, the human cells comprise human hematopoietic stem cells. In some embodiments, the human cells comprise human hematopoietic peripheral blood mononuclear cells.

In yet other aspects, the disclosure provides a method of producing the immunodeficient mouse of any one of the preceding claims, the method comprising breeding: (i) a mouse comprising an endogenous Kit allele comprising a null mutation, an exogenous nucleic acid encoding human interleukin 3 (huIL3), an exogenous nucleic acid encoding human granulocyte-macrophage colony-stimulating factor (huGM-CSF), and an exogenous nucleic acid encoding human Steel factor (huSCF); and (ii) a mouse comprising an endogenous H2-K allele comprising a null mutation, an endogenous H2-D allele comprising a null mutation, an endogenous H2-A allele comprising a null mutation, an exogenous nucleic acid encoding human interleukin 7 (huIL7), and an exogenous nucleic acid encoding human interleukin 15 (huIL15).

In some embodiments, the immunodeficient mouse is homozygous for the endogenous Kit allele comprising a null mutation. In some embodiments, the immunodeficient mouse is homozygous for the endogenous H2-K allele comprising a null mutation, homozygous for the endogenous H2-D allele comprising a null mutation, and/or homozygous for the endogenous H2-A allele comprising a null mutation.

Some aspects provide a method comprising: administering human cells to the immunodeficient mouse of any one of the preceding paragraphs.

In some embodiments, the method further comprises administering unfractionated human umbilical cord blood comprising the human cells.

In some embodiments, the human cells comprise human hematopoietic stem cells.

In some embodiments, the human cells comprise human peripheral blood mononuclear cells.

In some embodiments, the human cells are not enriched for CD34⁺ human hematopoietic stem cells.

In some embodiments, CD3⁺ human T cells have not been depleted from the human cells.

In some embodiments, the method excludes subjecting the immunodeficient mouse to myeloablation, optionally irradiation or chemical myeloablation.

Some aspects relate to an immunodeficient non-obese diabetic (NOD) mouse comprising: (a) an endogenous Il2rg allele comprising a null mutation, an endogenous Prkdc allele comprising a null mutation, and an endogenous Kit allele comprising a null mutation; and (b) a transgene encoding human interleukin 3 (huIL3), a transgene human granulocyte-macrophage colony-stimulating factor (huGM-CSF), and a transgene human Steel factor (huSCF).

In some embodiments, the immunodeficient mouse has been engrafted with human hematopoietic stem cells. In other embodiments, the immunodeficient mouse has been engrafted with human peripheral blood mononuclear cells.

In some embodiments, the immunodeficient mouse has been engrafted with diseased human cells.

In some embodiments, the diseased human cells are obtained from a subject having a genetic disorder.

In some embodiments, the genetic disorder is facioscapulohumeral muscular dystrophy (FSHD).

In some embodiments, the diseased human cells are human muscle cells.

In some embodiments, the human muscle cells are CD56+ muscle stem cells.

Other aspect relate to method comprising: administering human hematopoietic stem cells to a non-irradiated immunodeficient mouse of claim 1, wherein the human HSCs develop into innate immune cells; and administering human diseased cells to the non-irradiated immunodeficient mouse.

In some embodiments, about 10⁴ to about 10⁶ human HSCs are administered.

In some embodiments, the human diseased cells are administered about 4 to about 10 weeks post administration of the human HSCs.

In some embodiments, about 10⁴ to about 10⁶ human diseased cells, optionally muscle cells, further optionally CD56+ muscle stem cells, are administered.

In some embodiments, the human diseased cells are obtained from a subject having facioscapulohumeral muscular dystrophy (FSHD).

In some embodiments, the method further comprises administering a therapeutic modality to the immunodeficient mouse.

In some embodiments, the method further comprises assaying for a response of the innate immune cells to the human diseased cells.

In some embodiments, the response is an inflammatory response.

Some aspects relate to an immunodeficient non-obese diabetic (NOD) mouse comprising: (a) an endogenous Il2rg allele comprising a null mutation, an endogenous Prkdc allele comprising a null mutation, an endogenous H2-K allele comprising a null mutation (H2-K^{null}); an endogenous H2-D allele comprising a null mutation (H2-D^{null}); an endogenous H2-A allele comprising a null mutation (H2-A^{null}); and (b) a transgene encoding human interleukin 15 (huIL15). In some embodiments, a method herein comprises: administering human hematopoietic stem cells (HSCs) or human peripheral blood mononuclear cells (PBMCs) to the immunodeficient mouse, wherein the human HSCs develop into innate immune cells, optionally wherein the mouse is non-irradiated; and administering human diseased cells to the immunodeficient mouse. In some embodiments, about 10⁴ to about 10⁶ human HSCs or human PBMCs are administered. In some embodiments, the human diseased cells are administered about 4 to about 10 weeks post administration of the human HSCs or human PBMCs. In some embodiments, about 10⁴ to about 10⁶ human diseased cells are administered. In some embodiments, the method further comprises administering a therapeutic modality to the immunodeficient mouse. In some embodiments, the method further comprises assaying for a response of the innate immune cells to the human diseased cells. In some embodiments, the response is an inflammatory response.

Other aspects relate to an immunodeficient non-obese diabetic (NOD) mouse comprising: (a) an endogenous Il2rg allele comprising a null mutation and an endogenous Prkdc allele comprising a null mutation; and (b) a transgene encoding huIL15 and a transgene encoding human interleukin 7 (huIL7). In some embodiments, a method herein comprises: administering human HSCs or human PBMCs to the immunodeficient mouse, wherein the human HSCs develop into innate immune cells, optionally wherein the mouse is non-irradiated; and administering human diseased cells to the immunodeficient mouse. In some embodiments, about 10⁴ to about 10⁶ human HSCs or human PBMCs are administered. In some embodiments, the human diseased cells are administered about 4 to about 10 weeks post administration of the human HSCs or human PBMCs. In some embodiments, about 10⁴ to about 10⁶ human diseased cells are administered. In some embodiments, the method further comprises administering a therapeutic modality to the immunodeficient mouse. In some embodiments, the method further comprises assaying for a response of the innate immune cells to the human diseased cells. In some embodiments, the response is an inflammatory response.

### BRIEF DESCRIPTION OF DRAWINGS

FIGS. 1A-1E. NSG-SMG3-W41 mice support human hematopoietic stem cell (HSC) engraftment and selective expansion of innate immune cells but not T cells. FIG. 1A is a schematic showing the experimental design comparing the innate immune engraftment of NSG-SGM3 mice with or without 100 cGy irradiation to NSG-SGM3-W41 mice. FIG. 1B shows flow sorting assays of human CD45+ cells in blood at the indicated time points post HSC injection. FIGS. 1C-1E show flow cytometry analyses of the % of CD45+ cells that co-express CD33+ Myeloid cell marker, CD20+ B cell marker, and CD3⁺ T cell marker in blood at 4 weeks (FIG. 1C), 8 weeks (FIG. 1D) or 12 weeks (FIG. 1E) post HSC injection.

FIGS. 2A-2G. Co-xenoengraftment of human innate immune cells and skeletal muscle in NSG-SGM3-W41 mice. FIG. 2A is a schematic showing the co-engraftment protocol for human donor HSCs and FSHD and control muscle stem cells (myoblasts) and processing of tibialis anterior (TA) muscle xenografts for flow cytometry to identify cell lineages, RNA isolation for qPCR and NanoString, and sectioning for immunohistology. FIG. 2B is a table describing myoblast cell lines used. DUX4 expression levels previously described. FIG. 2C is a schematic describing HSC and muscle experimental groups for six HSC donors and three FSHD family cohorts. FIGS. 2D-2G show the percent hCD45+ cells in spleen (FIG. 2D), hCD45/CD20+ B cells (FIG. 2E), hCD45/CD33+ myeloid cells (FIG. 2F) or hCD45/CD3+ T cells (FIG. 2G) from the indicated engraftment conditions using flow cytometry assays. Each dot represents data from one mouse with bars showing mean per condition±SEM. Schematics in A and C were made using biorender.com.

FIGS. 3A-3C. Enhanced accumulation of human CD45+ innate immune cells in FSHD muscle xenografts. FIG. 3A shows humanized TA muscles were cryosectioned and immunostained with human-specific anti-CD45 to identify HSCs and Hoechst to identify nuclei. Representative images of FSHD and Control engrafted TA muscles are shown. Scale bar=50 μm. FIG. 3B shows quantification of CD45+ cells per TA muscle section for the indicated engraftment conditions. Each dot shows the number of human CD45+ cells in one muscle section with bars showing mean±SEM per condition. *=p<0.05, **=p<0.01, ***=p<0.001, ****=p<0.0001 by Welch's t-test. FIG. 3C shows serial sections immunostained with human-specific anti-CD45 to identify immune cells or spectrin β1 to identify human fibers and Hoechst and nuclei. The humanized muscle area is encircled with dotted lines based on the localization of spectrin β1 muscle fibers and is transposed onto the CD45 immunostained image. Scale bar=100 μm.

FIGS. 4A-4D. Enhanced accumulation of human B cells and macrophages in FSHD muscle xenografts. Humanized TA muscles were cryosectioned and co-immunostained with human-specific antibodies to CD19 to identify early B cells (FIG. 4A) or CD68 to identify macrophages (FIG. 4C) and Hoechst to show nuclei for FSHD or control engrafted TAs from mice engrafted with muscle cohorts 12, 15 or 17. Scale bars=50 μm. FIG. 4B is a quantification of B cell as shown by human CD19 immunostaining. Each dot represents one muscle section with bars showing mean per condition±SEM. FIG. 4D is a quantification of macrophages as shown by human CD68 immunostaining. Each dot represents one muscle section and is shown as mean per condition±SEM. *=p<0.05, **=p<0.01, ***=p<0.001, ****=p<0.0001 by Welch's t-test.

FIGS. 5A-5D. FSHD and unaffected control muscle stem cells engraft and differentiate in HSC engrafted NSG-SGM3-W41 mice. FIG. 5A shows humanized TA muscles were cryosectioned and co-stained with human-specific antibodies to lamin A/C to identify human nuclei, spectrin β1 to identify differentiated muscle fibers and Hoechst to identify all nuclei. Representative images of FSHD and control xenografts from cohorts 12, 15D1, 15D2, 17D3 and 17D4 are shown. Scale bar=100 μm. FIG. 5B shows quantification of spectrin β1 fibers for the indicated engraftment conditions. Each dot represents the number of fibers in one muscle section with bars showing mean per condition±SEM. *=p<0.05, **=p<0.01 by Welch's t-test. FIG. 5C shows representative images of FSHD and control xenografts from cohorts 12 and 15 with or without immune engraftment stained with spectrin β1 to identify differentiated muscle fibers. Scale bar=100 μm. FIG. 5D shows quantification of spectrin β1 fibers for the indicated engraftment conditions. Each dot represents the number of fibers in one muscle section with bars showing mean per condition±SEM. ***=p<0.001, ****=p<0.0001 by Welch's t-test.

FIGS. 6A-6D. Inflammatory response to FSHD muscle is immune donor dependent. Normalized NanoString counts for muscle genes (FIG. 6A), DUX4 target genes (FIG. 6B) and complement genes (FIG. 6C) as assayed in RNA isolated from immune/muscle engrafted TA muscles from the indicated cohorts. NanoString counts were log 2 transformed and the fold change of FSHD to control gene expression was calculated. Significant gene expression changes are denoted with an asterisk and were calculated using Welch's t-test. FIG. 6D shows NanoString log 2 transformed counts showing the expression of C3 RNA from individual TA muscles. Each dot represents expression data from one TA muscle with bars showing mean per condition±SEM. *=p<0.05, **=p<0.01, ****=p<0.0001 by Welch's t-test.

FIGS. 7A-7B. Human C3 localizes to FSHD and control human xenograft muscle fibers. FIG. 7A shows humanized TA muscles cryosectioned and co-stained with human-specific antibodies to C3 and spectrin β1 to identify differentiated muscle fibers and Hoechst to identify nuclei. Representative images of FSHD and control xenografts at low (top) and high magnification (bottom) are shown. Scale bar=(top) 100 μm, (bottom) 50 μm. FIG. 7B shows quantification of C3 puncta in spectrin β1+ fibers for FSHD and control engrafted TA muscle sections from cohorts 12, 15D1, 15D2, 17D3 and 17D4. Each dot represents the percent of fibers with greater than 10 C3 puncta with bars showing mean per condition±SEM. ***=p<0.001, ****=p<0.0001 by Welch's t-test.

FIGS. 8A-8C. Human CD45+ cells in spleen and xenograft muscle of mice engrafted with donor HSC cells. FIG. 8A shows representative flow data from HSC and muscle co-engrafted mice including gating strategy to identify human immune cell subtypes are shown. FIG. 8B shows flow cytometry assays from spleen for the percent human CD45+ cells. Animals that failed to develop human immune cell populations are included (no IM). Each dot represents data from one mouse with bars showing mean per condition±SEM. FIG. 8C shows quantification of CD45+ cells per section for the indicated engraftment conditions. Each dot represents the number of CD45+ cells in one muscle section with bars showing mean±SEM per condition.

FIG. 9 shows engraftment of cells (percent of total) 6 weeks after administration of unfractionated umbilical cord blood (UCB) to irradiated NSG-MHC DKO Tg(Hu-IL15) mice (n=10), irradiated NSG-MHC-class I/II KO mice (n=8), and irradiated NSG-Tg(Hu-IL7)(Hu-IL15) mice (n=10).

FIG. 10A shows results from a flow cytometry analysis of cells collected from blood 3 weeks after administration of unfractionated UCB to irradiated NSG-MHC DKO Tg(Hu-IL15) mice (n=13) and irradiated NSG-Tg(Hu-IL7)(Hu-IL15) mice (n=14). FIG. 10B shows engraftment of cells (percent of total) 3 weeks after administration of UCB. FIG. 10C shows results from a flow cytometry analysis of CD4 T cells and CD8 T cells 3 weeks after administration of UCB. FIG. 10D shows engraftment of CD4 T cells and CD8 T cells 3 weeks after administration of unfractionated UCB. FIG. 10E shows percentage of human CD45 cells 3, 6, and 9 weeks after administration of UCB. FIG. 10F shows percentage of human CD3 T cells (as a percentage of human CD45 cells) 3, 6, and 9 weeks after administration of UCB.

FIGS. 11A-11B show engraftment of cells (percent of total) 9 weeks after split cell injections into NSG-MHC DKO Tg(Hu-IL15) mice. FIG. 11C shows engraftment of human CD45 cells at 6 weeks and 9 weeks after split cell injections into NSG-MHC DKO Tg(Hu-IL15) mice.

### DETAILED DESCRIPTION

Humanized immunodeficient mouse models engrafted with human immune cells enable an in vivo evaluation of the efficacy and safety of human cell therapy products and other therapeutic products. Use of humanized mice, however, has been hindered by model-specific limitations, some of which include the development of graft versus host disease (GvHD), technical difficulty and cost associated with each humanized animal, and insufficient engraftment of some human immune cells. Even with the numerous mouse models that are currently available, there is still a great need in the field for a humanized model that is clinically relevant—a humanized model that can inform clinical applications of cancer immunotherapeutic agents, for example (see, e.g., Lee, et al., Dev Reprod., 2019, 2(2): 79-92; Morillon, et al., Anticancer Research, 2020, 40(10): 5329-5341, each of which are incorporated herein by reference). The present disclosure provides such a model.

### Mouse Models

Herein, for simplicity, reference is made to “mouse” and “mouse models” (e.g., surrogates for human conditions). It should be understood that these terms, unless otherwise stated, may be used interchangeably throughout the specification to encompass “rodent” and “rodent models,” including mouse, rat, and other rodent species.

It should also be understood that standard genetic nomenclature used herein provides unique identification for different rodent strains, and the strain symbol conveys basic information about the type of strain or stock used and the genetic content of that strain. Rules for symbolizing strains and stocks have been promulgated by the International Committee on Standardized Genetic Nomenclature for Mice. The rules are available on-line from the Mouse Genome Database (MGD; informatics.jax.org) and were published in print copy (Lyon et al. 1996). Strain symbols typically include a Laboratory Registration Code (Lab Code). The first Lab Code appended to a strain symbol identifies and credits the creator of the strain. The Lab Code at the end of a strain symbol indicates the current source for obtaining mice of that strain. Different Lab Codes appended to the same strain symbol distinguish sublines and alert the user that there may be genetic divergence between the different sublines. Lab Codes are assigned from a central registry to assure that each is unique. The registry is maintained at the Institute for Laboratory Animal Research (ILAR) at the National Academy of Sciences, Washington, D.C. Lab Codes may be obtained electronically at ILAR's web site (nas.edu/cls/ilarhome.nsf). See also Davisson M T, Genetic and Phenotypic Definition of Laboratory Mice and Rats/What Constitutes an Acceptable Genetic-Phenotypic Definition, National Research Council (US) International Committee of the Institute for Laboratory Animal Research. Washington (DC): National Academies Press (US); 1999.

As applied to a mutant mouse strain, “genetic background” or “background” refers to its genetic make-up (all its alleles at all loci) except the mutated gene of interest and a small amount of other genetic material, generally from one or two other strains. Correct strain nomenclature indicates what a mutant strain's background is. For example, the genetic background of the targeted mutant strains NOD.129S7(B6)-Rag1^{tm1Mom}/J (The Jackson Laboratory (JAX) Strain #003729) and NOD.Cg-Rag1^{tm1Mom} Prf1^{tm1Sdz}/SzJ (JAX Strain #004848) is primarily non-obese diabetic (NOD). However, the first strain carries a targeted mutation of the Rag1 gene, likely a few Rag1-linked alleles from 129S7-derived embryonic stem (ES) cells, and possibly some B6 alleles from crosses in its breeding history. In contrast, the second strain is a congenic (Cg) with more than one donor strain in its breeding history. It carries targeted mutations of the Rag1 and Prf1 genes and possibly some background alleles from those other strains.

A mouse model may be modified to enable the assessment of a disease. Any system (e.g., immune, respiratory, nervous, or circulatory), organ (e.g., blood, heart, blood vessels, spleen, thymus, lymph nodes, or lungs), tissue (e.g., epithelial, connective, muscle, and nervous), or cell type (e.g., lymphocytes or macrophages) may be modified, either independently or in combination, to enable studying disease in the models provided herein.

Three conventional methods used for the production of genome-modified mice (e.g., knockout mice, transgenic mice) include DNA microinjection (Gordon and Ruddle, Science 1981: 214: 1244-124, incorporated herein by reference), embryonic stem cell-mediated gene transfer (Gossler et al., Proc. Natl. Acad. Sci. 1986, 83: 9065-9069, incorporated herein by reference) and retrovirus-mediated gene transfer (Jaenisch, Proc. Natl. Acad. Sci. 1976, 73: 1260-1264, incorporated herein by reference), any of which may be used as provided herein. Genomic editing methods using, for example, clustered regularly interspace palindromic repeats (CRISPR/Cas) nucleases, transcription activator-like effector nucleases (TALENs), or zinc finger nucleases (ZFNs) may also be used and are described elsewhere herein.

Following delivery of nucleic acids to a fertilized embryo (e.g., a single-cell embryo (e.g., a zygote) or a multi-cell embryo (e.g., a developmental stage following a zygote, such as a blastocyst), the fertilized embryo is transferred to a pseudopregnant female, which subsequently gives birth to offspring. The presence or absence of a transgene of interest or a null mutation in an endogenous gene of interest, for example, may be confirmed using any number of genotyping methods (e.g., sequencing and/or genomic PCR).

New mouse models can also be created by breeding parental lines. With the variety of available mutant, knockout, knockin, transgenic, Cre-lox, Tet-inducible system, and other mouse strains, multiple mutations and transgenes may be combined to generate new mouse models. Multiple mouse strains may be bred together to generate double, triple, or even quadruple and higher multiple mutant/transgenic mice.

In some embodiments, parental mice are bred to produce F1 mice. A parental mouse may be, for example, homozygous, heterozygous, hemizygous, or homozygous null at a particular allele. Homozygous describes a genotype of two identical alleles at a given locus, heterozygous describes a genotype of two different alleles at a given locus, hemizygous describes a genotype consisting of only a single copy of a particular gene in an otherwise diploid organism, and homozygous null refers to an otherwise-diploid organism in which both copies of the gene are missing.

As is known in the art, immunodeficient mice have impaired or disrupted immune systems, such as specific deficiencies in MHC class I, II or both, B cell or T cell defects, or defects in both, natural killer (NK) cell defects, myeloid defects (e.g., defects in granulocytes and/or monocytes), macrophage defects, dendritic cell defects, as well as immunodeficiency due to knockdown of genes for cytokines, cytokine receptors, Toll-like receptors (TLR) and a variety of transducers and transcription factors of signaling pathways. Immunodeficiency mouse models include the single-gene mutation models such as nude-mice (nu) strains and the severe combined immunodeficiency (scid) strains, non-obese diabetic (NOD) strain, RAG (recombination activating gene) strains with targeted gene deletion and a variety of hybrids originated by crossing double and triple mutation mice strains with additional defects in innate and adaptive immunity.

An impaired immune system may be measured by any method known in the art including, but not limited to production of mature immune cells (e.g., B cells, T cells, dendritic cells, macrophages, natural killer cells), deficient endogenous cytokine signaling, limited resistance to infection, and reduced survival. In some embodiments, an immunodeficient mouse lacks mature mouse T cells, lacks mature mouse B cells, lacks functional mouse natural killer cells, and is deficient in endogenous (e.g., mouse) cytokine signaling. Mature T cells develop in the thymus and are released to other tissues, including blood, spleen, and lymphatic system. Mature B cells express pathogen-specific antibodies on their surface. Functional natural killer cells recognize and kill malignant and virally transformed cells without previously being exposed. Endogenous (e.g., mouse) cytokine signaling is important in maintaining homeostasis and relies on cytokines to regulate immune, nervous, and endocrine system function. Deficient endogenous (e.g., mouse) cytokine signaling means that the level of cytokine signaling is not sufficient to maintain immune system homeostasis compared to an endogenous immune system that is not deficient.

“Lack of” a particular cell type or signal (e.g., cytokine) in an immunodeficient mouse may be a complete absence of that cell type or signal, or it may be a substantial reduction of that cell type or signal, relative to a mouse that is not immunodeficient (e.g., has not been genetically modified to reduce innate immunity). For example, lack of a particular cell type (e.g., cell number) or signal (e.g., cytokine protein level) may be a reduction by at least 50%, at least 55%, at least 60%, at least 65%, at least 70%, at least 75%, at least 80%, at least 85%, at least 90%, at least 95%, or 100%, relative to a mouse that is not immunodeficient.

Lack a particular cell type may be assessed by any method known in the art including, but not limited to: flow cytometry; quantitative PCR (qPCR) of T cell markers (e.g., CD3, CD8, CD4, CD25, CD127, CD152), B cells markers (e.g., CD19, IgM, BCAP), and NK cells (e.g., CD224, CD122, NK1, NKp46, Ly49, CD11b, CD49b); immunofluorescence, and/or enzyme-linked immunosorbent assay (ELISA). Deficient cytokine signaling (e.g., mouse cytokine signaling) may be assessed by any method known in the art including, but not limited to: flow cytometry, qPCR of cytokines (e.g., IL2, IL7, IL15, IFNγ, IL4, IL5, IL9, IL13, IL25, IL17A, IL17F, IL22, TNFα, IL12, CCL3, GM-CSF, IL6, IL10, TGFβ, IL18, IL21), immunofluorescence, and/or ELISA.

Non-limiting examples of immunodeficient mouse model backgrounds that may be used herein include the following mouse strains:
Nude (nu) [Flanagan S P. Genet Res 1966; 8: 295-309; and Nehls M et al. Nature 1994; 372: 103-7];
Scid (scid) [Bosma G C et al. Nature 1983; 301:527-30; Mosier D E et al. Nature 1988; 335: 256-9; and Greiner D L et al. Stem Cells 1998; 16: 166-77];
NOD [Kikutani H et al. Adv Immunol 1992; 51: 285-322; and Anderson M S et al. Ann Rev Immunol 2005; 23: 447-85];
RAG1 and RAG2 (rag) [Mombaerts P et al. Cell 1992; 68: 869-77; Shinkai U et al. Cell 1992; 68: 855-67];
NOD-scid [Greiner D L et al. 1998; Shultz L D et al. J Immunol 1995; 154: 180-91; Melkus M W et al. Nature Med 2006; 12: 1316-22; and Denton P W et al. PLoS Med 2008; 4(12): e357];
IL2rg^{null} [DiSanto J P et al. Proc Natl Acad Sci USA 1995; 92: 377-81];
B2m^{null} [Christianson S W et al. J Immunol 1997; 158: 3578-86];
NOD-scid IL2rγ^{null} [Shultz L D et al. Nat Rev Immunol 2007; 7: 118-30; Ito M et al. Blood 2002; 100: 3175-82; Ishikawa I et al. Blood 2005; 106: 1565-73; and Macchiarini F et al. J Exp Med 2005; 202: 1307-11];
NOD-scid B2m^{null} [Shultz et al. 2007; Shultz L D et al. Transplantation 2003; 76: 1036-42; Islas-Ohlmayer M A et al. J Virol 2004; 78:13891-900; and Macchiarini et al. 2005];
HLA transgenic mice [Grusby M J et al. Proc Natl Acad Sci USA 1993; 90(9): 3913-7; and Roy C J et al. Infect Immun 2005; 73(4): 2452-60]. See, e.g., Belizario J E The Open Immunology Journal, 2009; 2:79-85;
NOG mice (NOD.cg-Prkdc^{scid}Il2rg^{tm1Sug}) [Shultz L D et al. Nat Rev Immunol 2007; 7: 118-30]; and
BRG mice (BALB/c;129S4-Rag2^{tm1.1Flv}) [Traggiai E et al. Science 2004; 304(5667): 104-107].

### Non-Obese Diabetic (NOD) Background

Provided herein, in some embodiments, are immunodeficient mouse models having the non-obese diabetic (NOD) background. The NOD mouse (e.g., JAX Strain #001976, NOD-Shi^{LtJ}) is a polygenic mouse model of autoimmune (e.g., Type 1) diabetes, characterized by hyperglycemia and insulitis, a leukocytic infiltration of the pancreatic islet cells. The NOD mouse is hypoinsulinemic and hyperglucagonemic, indicating a selective destruction of pancreatic islet beta cells. The major component of diabetes susceptibility in a NOD mouse is the unique MHC haplotype. A NOD mouse also exhibits multiple aberrant immunophenotypes including defective antigen presenting cell immunoregulatory functions, defects in the regulation of the T lymphocyte repertoire, defective NK cell function, defective cytokine production from macrophages (Fan et al., 2004) and impaired wound healing. It also lacks hemolytic complement, C5. A NOD mouse also is severely hard-of-hearing. A variety of mutations causing immunodeficiencies, targeted mutations in cytokine genes, as well as transgenes affecting immune functions, have been backcrossed into the NOD inbred strain background.

In some aspects of the present disclosure, an immunodeficient mouse provided herein based on the NOD background has a genetic background (“background”) selected from NOD-Cg.-Prkdc^{scid}IL2rg^{tm1wJI}/SzJ (NSG™), a NOD.Cg-Rag1^{tm1Mom} Il2rg^{tm1Wjl}/SzJ (NRG), NOD.Cg-Prkdc^{scid}Il2rg^{tm1Sug}/ShiJic (NOG), NOD-Prkdc^{em26Cd52}Il2rg^{em26Cd22}/NjuCrl (NCG), and BALB/c-Rag2^{tm1Fwa} Ilr2g^{tm1Sug}/JicTac (BRG). Other immunodeficient mouse strains are contemplated herein.

In some embodiments, an immunodeficient mouse model based on the NOD background has an NOD-Cg.-Prkdc^{scid}IL2rg^{tm1wJl}/SzJ (NSG®) genetic background. The NSG® mouse (e.g., JAX Strain #005557) is an immunodeficient mouse that lacks mature T cells, B cells, and NK cells, is deficient in multiple cytokine signaling pathways, and has many defects in innate immune immunity (see, e.g., Shultz, Ishikawa, & Greiner, 2007; Shultz et al., 2005; and Shultz et al., 1995, each of which is incorporated herein by reference). The NSG® mouse, derived from the NOD mouse strain NOD/ShiLtJ (see, e.g., Makino et al., 1980, which is incorporated herein by reference), includes the Prkdc^{scid} mutation (also referred to as the “severe combined immunodeficiency” mutation or the “scid” mutation) and the Il2rg^{tm1Wjl} targeted mutation. The Il2rg^{tm1Wjl} mutation is a null mutation in the gene encoding the interleukin 2 receptor gamma chain (Il2rg, homologous to IL2RG in humans), which blocks NK cell differentiation, thereby removing an obstacle that prevents the efficient engraftment of primary human cells (Cao et al., 1995; Greiner et al., 1998; and Shultz et al., 2005, each of which is incorporated herein by reference).

In some embodiments, an immunodeficient mouse model has an NRG background. The NRG mouse (e.g., JAX Strain #007799) is extremely immunodeficient. This mouse comprises two mutations on the NOD/ShiLtJ genetic background; a targeted knockout mutation in recombination activating gene 1 (Rag1) and a complete null allele of the IL2 receptor common gamma chain (IL2rg^{null}). The extreme immunodeficiency of NRG allows the mice to be humanized by engraftment of human CD34⁺ hematopoietic stem cells (HSC) and patient derived xenografts (PDXs) at high efficiency. The immunodeficient NRG mice are more resistant to irradiation and genotoxic drugs than mice with a scid mutation in the DNA repair enzyme Prkdc.

In some embodiments, an immunodeficient mouse model is an NOG mouse. The NOG mouse (Ito M et al., Blood 2002) is an extremely severe combined immunodeficient (scid) mouse established by combining the NOD/scid mouse and the IL2 receptor-T chain knockout (IL2rγKO) mouse (Ohbo K. et al., Blood 1996). The NOG mouse lacks T and B cells, lacks natural killer (NK) cells, exhibits reduced dendritic cell function and reduced macrophage function, and lacks complement activity.

In some embodiments, an immunodeficient mouse model has an NCG background. The NCG mouse (e.g., Charles River Stock #572) was created by sequential CRISPR/Cas9 editing of the Prkdc and Il2rg loci in the NOD/Nju mouse, generating a mouse coisogenic to the NOD/Nju. The NOD/Nju carries a mutation in the Sirpa (SIRPα) gene that allows for engrafting of foreign hematopoietic stem cells. The Prkdc knockout generates a SCID-like phenotype lacking proper T-cell and B-cell formation. The knockout of the Il2rg gene further exacerbates the SCID-like phenotype while additionally resulting in a decrease of NK cell production.

In some embodiments, an immunodeficient mouse model has a BRG background. The BRG mouse (BALB/c-Rag2^{tm1Fwa} Ilr2g^{tm1Sug}/JicTac) (e.g., Taconic #11503) was created by mating BALB/cA-Rag2 knock-out (Rag2^{tm1Fwa}) mice with BALB/cA-Il2rg (Ilr2g^{tm1Sug}) mice. (Traggiai E. et al., Science 2004). The BRG mouse lacks mature T, B, and NK cells, does not produce imnunoglobulins, and exhibits reduced dendritic cell function (relative to wild-type BALB/c mice).

### Interleukin 2 Receptor Gamma (Il2rg) Allele

This gene encodes a transmembrane protein that is a common subunit of several interleukin receptor complexes. These receptors are comprised of alpha and beta subunits in addition to this gamma subunit. Signaling through this pathway in important in immune cell differentiation and function. Provided herein, in some embodiments, are immunodeficient mouse models comprising an endogenous interleukin 2 receptor, gamma chain (Il2rg) allele comprising a null mutation (Il2rg^{null}). Examples of Il2rg^{null} alleles include the Il2rg^{tm1Wjl} allele (Cao X, et al., Immunity. 1995 March; 2(3):223-38) and the Il2rg^{tm1Sug} allele (Ohbo K, et al., Blood. 1996 Feb. 1; 87(3):956-67). In some embodiments, an immunodeficient mouse comprises the Il2rg^{tm1Wjl} allele. In some embodiments, an immunodeficient mouse comprises the Il2rg^{tm1Sug} allele. In some embodiments, an immunodeficient mouse comprises the Il2rgem26Cd22 allele. Mouse IL2Rγ is encoded by the mouse Il2rg gene (Gene ID: 16186). The human ortholog of mouse IL2Rγ is interleukin 2 receptor subunit gamma (IL2RG).

### Protein Kinase, DNA-Activated, Catalytic Subunit (Prkdc) Allele

This gene enables DNA-dependent protein kinase activity, double-stranded DNA binding activity, and enzyme binding activity. It is involved in several processes, including regulation of cellular protein metabolic process, regulation of hematopoietic stem cell differentiation, and regulation of hemopoiesis. It acts upstream of or within several processes, including DNA metabolic process, ectopic germ cell programmed cell death, and immune system development. Provided herein, in some embodiments, are immunodeficient mouse models comprising an endogenous protein kinase, DNA activated, catalytic polypeptide (Prkdc) allele comprising a null mutation (Prkdc^{null}) In some embodiments, the null mutation in the endogenous Prkdc allele is a severe combined immunodeficiency (scid) mutation. Examples of Prkdc^{null} alleles include the Prkdc^{scid} allele, commonly referred to as scid (Bosma G C, et al., Nature. 1983 Feb. 10; 301(5900):527-30) and Prkdc^{em26Cd52}. In some embodiments, an immunodeficient mouse comprises the Prkdc^{scid} allele. In some embodiments, an immunodeficient mouse comprises the Prkdc^{em26Cd52} allele. Mouse PRKDC is encoded by the mouse Prkdc gene (Gene ID: 19090). The human ortholog of mouse PRKDC is protein kinase, DNA-activated, catalytic subunit (PRKDC).

In some embodiments, an immunodeficient mouse model has a NOD.Cg-Prkdc^{scid} d Il2rg^{tm1Wjl}/SzJ background (also referred to as NSG®; NOD-scid IL2Rgamma^{null}; NOD-scid IL2Rg^{null}; and NOD scid gamma) (JAX Strain #005557). These mice are extremely immunodeficient. The mice carry two mutations on the NOD/ShiLtJ genetic background; severe combined immune deficiency (scid) and a complete null allele of the IL2 receptor common gamma chain (IL2rg^{null}). The scid mutation is in the DNA repair complex protein PRKDC and renders the mice B and T cell deficient. The IL2rg^{null} mutation prevents cytokine signaling through multiple receptors, leading to a deficiency in functional NK cells. The severe immunodeficiency allows the mice to be humanized by engraftment of human CD34⁺ HSCs, PBMCs, patient derived xenografts (PDX), or adult stem cells and tissues.

### Recombination Activating Gene 1 (Rag1) Allele

This gene enables several functions, including protein homodimerization activity, ubiquitin protein ligase activity, and zinc ion binding activity. It is involved in immune system development, positive regulation of T cell differentiation and protein ubiquitination. It acts upstream of or within several processes, including T cell homeostasis, hematopoietic or lymphoid organ development, and negative regulation of apoptotic process. Provided herein, in some embodiments, are immunodeficient mouse models comprising an endogenous recombination activating gene 1 (Rag1) allele comprising a null mutation (Rag1^{null}) Examples of Rag1^{null} alleles include: Rag1^{tm1Mom} (Mombaerts P, et al., Cell. 1992 Mar. 6; 68(5):869-77), Rag1^{em1Gpt}, Rag1^{em1Ldn}, Rag1^{em2Gpt}, Rag1^{em28Gpt}, Rag1^{m1Btlr}, Rag1^{tm1(cre)Thr}, Rag1^{tml(GFP)lmku}, Rag1^{tm1Fwa}, Rag1^{tm1Jsek}, Rag1^{m1Anu}, Rag1^{tm1.1Sadu}, Rag1^{tm1Mnz}, Rag1^{coln}, Rag1^{em1Dgs}, Rag1^{em2Dgs}, Rag1^{em2Ldn}, Rag1^{em2Tutzy}, Rag1^{em3Ldn}, Rag1^{em4Ldn}, Rag1^{em5Lutzy}, Rag1^{em8Lutzy}, Rag1^{em10Lutzy} Rag1^{m2Btlr}, Rag1^{m3Btlr}, Rag1^{m4Btlr}, Rag1^{tm1Bal}, Rag1^{tm1Jmj}, Rag1^{tm1Nju}, Rag1^{tm1Smoc}, Rag1^{tm1a(KOMP)Wtsi}, and Rag1^{tm1e(KOMP)Wtsi}. In some embodiments, an immunodeficient mouse comprises the Rag1^{tm1Mom} allele. Mouse RAG1 is encoded by the mouse Rag1 gene (Gene ID: 19373). The human ortholog of mouse RAG1 is recombination activating 1 (RAG1).

### Recombination Activating Gene 2 (Rag2) Allele

This gene enables several functions, including phosphatidylinositol phosphate binding activity, phosphatidylinositol-3,4-bisphosphate binding activity, and zinc ion binding activity. Involved in V(D)J recombination and pre-B cell allelic exclusion. It acts upstream of or within several processes, including B cell homeostatic proliferation, lymphocyte differentiation, and positive regulation of organ growth. Provided herein, in some embodiments, are immunodeficient mouse models comprising an endogenous recombination activating gene 2 (Rag2) allele comprising a null mutation (Rag2^{null}). Examples of Rag2^{null} alleles include: Rag2^{tm1Fwa} (Shinkai Y, et al., Cell. 1992 Mar. 6; 68(5):855-67), Rag2^{tm1Mnz}, Rag2^{m1Btlr}, Rag2^{tm1.1Cgn}, Rag2^{tm1.1Desi}, Rag2^{tm1Avla}, Rag2^{tm1cgn}, Rag2^{tm1Mao}, Rag1^{tm1Libo}, and Rag2^{tm1Tgi}. In some embodiments, an immunodeficient mouse comprises the Rag2^{tm1Fwa} allele. Mouse RAG2 is encoded by the mouse Rag2 gene (Gene ID: 5897). The human ortholog of mouse RAG2 is recombination activating 2 (RAG2).

### MHC Class I/Class II Alleles

The Major Histocompatibility Complex (MHC) genomic region harbors duplicated genes that express protein molecules responsible for the rejection of transplanted tissue, restricted antigen presentation and the recognition of self and non-self. The Mhc genomic region in the mouse, located on chromosome 17, is named H2 and the genes within this region are usually classified into three distinct classes (I to III) based on their structure and function. The class I molecules generally elicit immune responses by presenting peptide antigens derived from intracellular proteins to T lymphocytes. The class II molecules play a central role in the selection of the T cell repertoire, in the establishment and regulation of the adaptive immune response, and in autoimmune deviation. Provided herein, in some embodiments, are immunodeficient mouse models comprising an endogenous histocompatibility 2, K region (H2-K) allele comprising a null mutation (H2-K^{null}). Examples of H2-K^{null} alleles include: H2-K1^{tm1Bpe} (Perarnau B, et al., Eur J Immunol. 1999 April; 29(4):1243-52), H2-K^{bm1}, H2-K^{bm3}, H2-K^{bm4}, H2-K^{bm5}, H2-K^{bm8}, H2-K^{bm10}, H2-K^{bm11}, H2-K^{bm16}, H2-K^{bm23}, H2-K^{bm29}, H2-K^{dm4}, H2-K^{dm5}, H2-K^{em1(HLA-A*0201)Gpt}, Tg(GFAP-B2m, GFAP-H2-K^{b})1Gjh, Tg(H2-K)1Alm, Tg(H2-K^{b})1Rms, Tg(HLA-A2/H2-K)1Scr, Tg(HLA-A/H2-K)1Chmb, and Tg(HLA-B/H2-K)1Chmb. In some embodiments, an immunodeficient mouse model comprises an endogenous histocompatibility 2, K1, K region (H2-K1) allele comprising a null mutation (H2-K1^{null}). In some embodiments, an immunodeficient mouse model comprises a H2-K1^{tm1Bpe} allele. The human ortholog of mouse H2-K1 is major histocompatibility complex, class I, A (HLA-A).

Provided herein, in some embodiments, are immunodeficient mouse models comprising an endogenous H2-D allele comprising a null mutation (H2-D^{null}). Examples of H2-D^{null} alleles include: H2-D1^{tm1Bpe} (Pascolo S, et al., J Exp Med. 1997 Jun. 16; 185(12):2043-51), H2-D^{dm1}, Tg(H2-D^{b})2Bujf, Tg(H2-D^{d})28Bee, Tg(H2-D^{d})D8Gja, Tg(H2-D^{d}/H2-L^{d})DL1U1, and Tg(HLA-A24/H2-D/B2M)3DVs. In some embodiments, an immunodeficient mouse model comprises an endogenous histocompatibility 2, D1, locus 1 (H2-D1) allele comprising a null mutation (H2-D1^{null}). In some embodiments, an immunodeficient mouse model comprises a H2-D1^{tm1Bpe} allele. The human ortholog of mouse H2-D1 is major histocompatibility complex, class I, A (HLA-A).

Provided herein, in some embodiments, are immunodeficient mouse models comprising an endogenous H2-A allele comprising a null mutation (H2-A^{null}). Examples of H2-A^{null} alleles include: H2-Ab1^{em1Mvw} (Brehm M A, et al., FASEB J. 2019 March; 33(3):3137-3151), H2-Ab^{bm12}; H2-Ab1^{em1(HLA-DPB1)Smoc}; H2-Ab1^{em1Dys}; H2-Ab1^{em1Gpt}; H2-Ab1^{em1Ygch}; H2-Ab1^{em2Gpt}; H2-Ab1^{em2smoc}; H2-Ab1^{em2Ygch}; H2-Ab1^{em3Smoc}; H2-Ab1^{em4Smoc}; H2-Ab1^{em22Gp}; H2-Ab1^{tm1,Jssh}; H2-Ab1^{tm1,Koni}; H2-Ab1^{Tm1,1Sish}; H2-Ab1^{tm1Doi}; H2-Ab1^{Tm1Gru}; H2-Ab1^{tmJP1}; H2-Ab1^{Tm1Hp1}; H2-Ab1^{tm1Koni}; H2-Ab1^{tm1Wag}; H2-Ab1^{tm2Hpl}; H2^{em1Gpt}; H2^{em1Smoc}; Tg(Ab1TL)1Gru; Tg(CD2-CD4,HLA-DQA1,HLA-DQB1)1Ell; Tg(CD4,HLA-DQA1,HLA-1DQB1)N8Ell; Tg(68-H2-Ab^{o})#Rhd; Tg(H2-Ab1)62Dim; Tg(H2-Ab1)GBQRhd; Tg(H2-Ea-H2-Ab1)1Pmr; Tg(HLA-DQA1,HLA-DQB1)70Myl; Tg(HLA-DQA1,HLA-DQB1_73Myl, Tg(KRT14-H2-Ab1)1Glm; H2-Ab1^{tm1a(EUCOMM)Hmgu}; H2-Ab1^{tm1e(EUCOMM)Hmgu}; and H2-Ab1^{tm2e(EUCOMM)Hmgu}. In some embodiments, an immunodeficient mouse model comprises an endogenous H2-Ab1 allele comprising a null mutation (H2-Ab1^{null}). In some embodiments, an immunodeficient mouse model comprises a H2-Ab1^{em1Mvw} allele. The human ortholog of mouse H2-Ab1 is major histocompatibility complex, class II, DQ beta 1 (HLA-DQB1).

Provided herein, in some embodiments, are immunodeficient mouse models comprising an endogenous mouse H2-K allele comprising a null mutation, an endogenous mouse H2-D allele comprising a null mutation, and an endogenous mouse H2-A allele comprising a null mutation. In some embodiments, the endogenous H2-K1 allele comprising a null mutation is a H2-K1^{tm1Bpe} allele, the endogenous H2-D allele comprising a null mutation is a H2-D1^{tm1Bpe} allele, and the endogenous H2-A allele comprising a null mutation is an H2-Ab1^{em1Mvw} allele.

In some embodiments, an immunodeficient mouse model has a NOD.Cg-Prkdc^{scid} H2-K1^{tm1Bpe} H2-Ab1^{em1Mvw} H2-D1^{tm1Bpe} Il2rg^{tm1Wjl}/SzJ background (also referred to as NSG-MHC I/II DKO; NSG-(K^{b}D^{b})^{null} (IA)^{null}; and NSG-(K^{b} D^{b})^{null} (IA^{null})) (JAX Strain #025216). NSG-MHC I/II DKO mutant mice combine the features of the severe combined immune deficiency mutation (scid), IL2 receptor gamma chain deficiency, MHC class I molecule deficiency (H2-K and D), and MHC class II molecule deficiency (IA) and exhibit a significant delay in the onset of GvHD.

### Kit Allele

This gene (c-Kit proto-oncogene) is the cellular homolog of the transforming gene of a feline retrovirus (v-Kit). The protein includes characteristics of a protein kinase transmembrane receptor. Provided herein, in some embodiments, are immunodeficient mouse models comprising an endogenous KIT proto-oncogene receptor tyrosine kinase (Kit) allele comprising a null mutation (Kit^{null}). In some embodiments, the mutation in the Kit allele is a spontaneous mutation. Examples of Kit^{null} alleles include: Kit^{W-41J} (Cosgun et al., Cell Stem Cell, 2014, 15(2): 227-238); Del(5Kit-Cepl35)1Utr; In(5)9Rk; In(5)30Rk; In(5)33Rk; Kit^{W-19H}; Kit^{W-57J}, Kit^{W-18J}, Kit^{W-sh}, Kit^{W-43J}, Kit^{W-34J}, Kit^{W-55J}, Kit^{W-35J}, and Kit^{W-39J}. In some embodiments, an immunodeficient mouse comprises the Kit^{W-41J} allele. Mouse KIT is encoded by the mouse Kit gene (Gene ID: 16590). The human ortholog of mouse KIT is KIT proto-oncogene, receptor tyrosine kinase (KIT).

### Human Cytokines

The immunodeficient mouse models provided herein, in some embodiments, express several exogenous nucleic acids (e.g., transgenes), each encoding a human cytokine. In some embodiments, an immunodeficient mouse model comprises an exogenous nucleic acid encoding human interleukin 7 (huIL7). In some embodiments, an immunodeficient mouse model comprises an exogenous nucleic acid encoding human interleukin 15 (huIL15). In some embodiments, an immunodeficient mouse model comprises an exogenous nucleic acid encoding human interleukin 3 (huIL3). In some embodiments, an immunodeficient mouse model comprises an exogenous nucleic acid encoding human granulocyte-macrophage colony-stimulating factor (huGM-CSF). In some embodiments, an immunodeficient mouse model comprises an exogenous nucleic acid encoding human Steel (huSCF). In some embodiments, an immunodeficient mouse model comprises an exogenous nucleic acid encoding huIL1, an exogenous nucleic acid encoding huIL15, an exogenous nucleic acid encoding huIL3, an exogenous nucleic acid encoding huGM-CSF, and an exogenous nucleic acid encoding huSCF. Thus, in some embodiments, cells of an immunodeficient mouse model express huIL1, express huIL15, express huIL3, express huGM-CSF, and express huSCF.

### Human IL7

In some embodiments, an immunodeficient mouse provided herein expresses human interleukin 7 (huIL7). Interleukin 7 is a cytokine critical in B and T cell development. IL7 binds hepatocyte growth factor to promote B cell precursor growth and stimulates V(D)J rearrangement of the T cell receptor. IL7 also plays a role in lymphoid cell survival and in maintenance and development of naive and memory T cells.

A human IL7 sequence may be any human IL7 sequence known in the art (see, e.g., Gene ID: 3574). In some embodiments, a human IL7 sequence is codon-optimized for expression in a non-human host (e.g., immunodeficient mouse). A human IL7 sequence may be expressed (e.g., in a mouse cell) by any method provided herein.

### Human IL15

In some embodiments, an immunodeficient mouse provided herein expresses human interleukin 15 (huIL15). Interleukin 15 is a cytokine that regulates T cell and natural killer cell activation and proliferation by binding hematopoietin receptor to stimulate cell differentiation and regulating the number of CD8⁺ T cells.

A human IL15 sequence may be any human IL15 sequence known in the art (see, e.g., Gene ID: 3600). In some embodiments, a human IL15 sequence is codon-optimized for expression in a non-human host (e.g., immunodeficient mouse). A human IL15 sequence may be expressed (e.g., in a mouse cell) by any method provided herein.

### Human IL3

In some embodiments, an immunodeficient mouse provided herein expresses human interleukin 3 (huIL3). Interleukin 3 is a cytokine that promotes the growth and proliferation of a broad range of hematopoietic cell types, including granulocytes, monocytes, and dendritic cells. IL3 is produced by activated T cells and stimulates the differentiation of immature myelomonocytic cells to alter the macrophage and granulocyte cell populations.

A human IL3 sequence may be any human IL3 sequence known in the art (see, e.g., Gene ID: 3562). In some embodiments, a human IL3 sequence is codon-optimized for expression in a non-human host (e.g., immunodeficient mouse). A human IL3 sequence may be expressed (e.g., in a mouse cell) by any method provided herein.

### Human GM-CSF

In some embodiments, an immunodeficient mouse provided herein expresses human granulocyte-macrophage colony-stimulating factor (huGM-CSF). GM-CSF is a cytokine that regulates macrophage and granulocyte differentiation, dendritic cell development, and the maintenance of homeostasis.

A human GM-CSF sequence may be any human GM-CSF sequence known in the art (see, e.g., Gene ID: 1437). In some embodiments, a human GM-CSF sequence is codon-optimized for expression in a non-human host (e.g., immunodeficient mouse). A human GM-CSF sequence may be expressed (e.g., in a mouse cell) by any method provided herein.

### Human SCF

In some embodiments, an immunodeficient mouse provided herein expresses human Steel factor (SCF, SF, or KITLG). SCF, also referred to as KIT ligand (KITLG), is a ligand of the tyrosine-kinase receptor encoded by the KIT locus. This ligand is a pleiotropic factor that acts in utero in germ cell and neural cell development, and hematopoiesis, all believed to reflect a role in cell migration. In adults, it functions pleiotropically, while mostly noted for its continued requirement in hematopoiesis.

A human SCF sequence may be any human SCF sequence known in the art (see, e.g., Gene ID: 4254). In some embodiments, a human SCF sequence is codon-optimized for expression in a non-human host (e.g., immunodeficient mouse). A human SCF sequence may be expressed (e.g., in a mouse cell) by any method provided herein.

### Exemplary Humanized Immunodeficient Mouse Model

### Immunodeficient Mouse Expressing Human IL7 Transgene

In some embodiments, the genome of an immunodeficient mouse model of the present disclosure comprises an exogenous nucleic acid encoding human interleukin-7 (huIL7) (e.g., a human IL7 transgene). In some embodiments, the genome of the mouse further comprises an endogenous Il2rg allele comprising a null mutation (Il2rg^{null}). In some embodiments, the genome of the mouse further comprises an endogenous Prkdc allele comprising a null mutation (Prkdc^{null}). In some embodiments, the genetic background of the mouse is NOD.Cg-Prkd^{scid} Il2rg^{tm1Wjl}/SzJ. In some embodiments, the immunodeficient mouse model has been engrafted with human cells selected from peripheral blood mononuclear cells (PBMCs), hematopoietic stem cells (HSCs), and umbilical cord blood (UCB) cells (e.g., unfractionated human UCB cells). In some embodiments, the mouse has been irradiated. In some embodiments, a method of the disclosure comprises administering the human cells to the mouse. In some embodiments, a method further comprises irradiating the mouse prior to engrafting the human cells.

### Immunodeficient Mouse Expressing Human IL7 and Human IL15 Transgenes

In some embodiments, the genome of an immunodeficient mouse model of the present disclosure comprises an exogenous nucleic acid encoding human interleukin-7 (huIL7) (e.g., a huIL7 transgene) and an exogenous nucleic acid encoding human interleukin-15 (huIL15) (e.g., a huIL15 transgene). In some embodiments, the genome of the mouse further comprises an endogenous Il2rg allele comprising a null mutation (Il2rg^{null}). In some embodiments, the genome of the mouse further comprises an endogenous Prkdc allele comprising a null mutation (Prkdc^{null}). In some embodiments, the genetic background of the mouse is NOD.Cg-Prkdc^{scid} Il2rg^{tm1Wjl}/SzJ.

In some embodiments, the immunodeficient mouse model has been engrafted with human cells selected from PBMCs, HSCs, and UCB cells (e.g., unfractionated human UCB cells). In some embodiments, the mouse has been irradiated. In some embodiments, a method of the disclosure comprises administering the human cells to the mouse. In some embodiments, a method further comprises irradiating the mouse prior to engrafting the human cells.

### Immunodeficient Mouse Deficient in MHC and Expressing Human IL15 Transgene

In some embodiments, the genome of an immunodeficient mouse model of the present disclosure comprises: an endogenous H2-K allele comprising a null mutation (H2-K^{null}); an endogenous H2-D allele comprising a null mutation (H2-D^{null}); an endogenous H2-A allele comprising a null mutation (H2-A^{null}); and an exogenous nucleic acid encoding huIL15 (e.g., a huIL15 transgene). In some embodiments, the genome of the mouse further comprises an endogenous Il2rg allele comprising a null mutation (Il2rg^{null}). In some embodiments, the genome of the mouse further comprises an endogenous Prkdc allele comprising a null mutation (Prkdc^{null}). In some embodiments, the genetic background of the mouse is NOD.Cg-Prkd^{scid} Il2rg^{tm1Wjl}/SzJ.

In some embodiments, the immunodeficient mouse model has been engrafted with human cells selected from PBMCs, HSCs, and UCB cells (e.g., unfractionated human UCB cells). In some embodiments, the mouse has been irradiated. In some embodiments, a method of the disclosure comprises administering the human cells to the mouse. In some embodiments, a method further comprises irradiating the mouse prior to engrafting the human cells.

### Immunodeficient Mouse Deficient in MHC and Expressing Human IL7 and Human IL15 Transgenes

In some embodiments, the genome of an immunodeficient mouse model of the present disclosure comprises: an endogenous H2-K allele comprising a null mutation (H2-K^{null}); an endogenous H2-D allele comprising a null mutation (H2-D^{null}); an endogenous H2-A allele comprising a null mutation (H2-A^{null}); an exogenous nucleic acid encoding huIL7 (e.g., a huIL7 transgene); and an exogenous nucleic acid encoding huIL15 (e.g., a huIL15 transgene). In some embodiments, the genome of the mouse further comprises an endogenous Il2rg allele comprising a null mutation (Il2rg^{null}). In some embodiments, the genome of the mouse further comprises an endogenous Prkdc allele comprising a null mutation (Prkdc^{null}). In some embodiments, the genetic background of the mouse is NOD.Cg-Prkdc^{scid} Il2rg^{tm1Wjl}/SzJ.

In some embodiments, the immunodeficient mouse model has been engrafted with human cells selected from PBMCs, HSCs, and UCB cells (e.g., unfractionated human UCB cells). In some embodiments, the mouse has been irradiated. In some embodiments, a method of the disclosure comprises administering the human cells to the mouse. In some embodiments, a method further comprises irradiating the mouse prior to engrafting the human cells.

### Immunodeficient Mouse Deficient in MHC and Expressing Human IL3, Human GM-CSF, Human SCF, and Human IL15 Transgenes

In some embodiments, the genome of an immunodeficient mouse model of the present disclosure comprises: an endogenous H2-K allele comprising a null mutation (H2-K^{null}); an endogenous H2-D allele comprising a null mutation (H2-D^{null}); an endogenous H2-A allele comprising a null mutation (H2-A^{null}); an exogenous nucleic acid encoding human interleukin-3 (huIL3); an exogenous nucleic acid encoding human granulocyte-macrophage colony-stimulating factor (huGM-CSF); and an exogenous nucleic acid encoding human Steel factor (huSCF); and an exogenous nucleic acid encoding huIL15 (e.g., a huIL15 transgene). In some embodiments, the genome of the mouse further comprises an endogenous Il2rg allele comprising a null mutation (Il2rg^{null}). In some embodiments, the genome of the mouse further comprises an endogenous Prkdc allele comprising a null mutation (Prkdc^{null}). In some embodiments, the genetic background of the mouse is NOD.Cg-Prkd^{scid} Il2rg^{tm1Wjl}/SzJ.

In some embodiments, the immunodeficient mouse model has been engrafted with human cells selected from PBMCs, HSCs, and UCB cells (e.g., unfractionated human UCB cells). In some embodiments, the mouse has been irradiated. In some embodiments, a method of the disclosure comprises administering the human cells to the mouse. In some embodiments, a method further comprises irradiating the mouse prior to engrafting the human cells.

### Immunodeficient Mouse Deficient in MHC and Expressing Human IL3, Human GM-CSF, Human SCF, and Human IL7 Transgenes

In some embodiments, the genome of an immunodeficient mouse model of the present disclosure comprises: an endogenous H2-K allele comprising a null mutation (H2-K^{null}); an endogenous H2-D allele comprising a null mutation (H2-D^{null}); an endogenous H2-A allele comprising a null mutation (H2-A^{null}); an exogenous nucleic acid encoding huIL3; an exogenous nucleic acid encoding huGM-CSF; and an exogenous nucleic acid encoding huSCF; and an exogenous nucleic acid encoding huIL7 (e.g., a huIL7 transgene). In some embodiments, the genome of the mouse further comprises an endogenous Il2rg allele comprising a null mutation (Il2rg^{null}). In some embodiments, the genome of the mouse further comprises an endogenous Prkdc allele comprising a null mutation (Prkdc^{null}). In some embodiments, the genetic background of the mouse is NOD.Cg-Prdc^{scid} Il2rg^{tm1Wjl}/SzJ.

In some embodiments, the immunodeficient mouse model has been engrafted with human cells selected from PBMCs, HSCs, and UCB cells (e.g., unfractionated human UCB cells). In some embodiments, the mouse has been irradiated. In some embodiments, a method of the disclosure comprises administering the human cells to the mouse. In some embodiments, a method further comprises irradiating the mouse prior to engrafting the human cells.

### Immunodeficient Mouse Deficient in MHC and Expressing Human IL3, Human GM-CSF, Human SCF, Human IL7, and Human IL1S Transgenes

In some embodiments, the genome of an immunodeficient mouse model of the present disclosure comprises: an endogenous H2-K allele comprising a null mutation (H2-K^{null}); an endogenous H2-D allele comprising a null mutation (H2-D^{null}); an endogenous H2-A allele comprising a null mutation (H2-A^{null}); an exogenous nucleic acid encoding huIL3; an exogenous nucleic acid encoding huGM-CS; and an exogenous nucleic acid encoding huSCF; an exogenous nucleic acid encoding huIL7 (e.g., a huIL7 transgene); and an exogenous nucleic acid encoding human huIL15 (e.g., a huIL15 transgene). In some embodiments, the genome of the mouse further comprises an endogenous Il2rg allele comprising a null mutation (Il2rg^{null}). In some embodiments, the genome of the mouse further comprises an endogenous Prkdc allele comprising a null mutation (Prkdc^{null}). In some embodiments, the genetic background of the mouse is NOD.Cg-Prkdc^{scid} Il2rg^{tm1Wi1}/SzJ.

In some embodiments, the immunodeficient mouse model has been engrafted with human cells selected from PBMCs, HSCs, and UCB cells (e.g., unfractionated human UCB cells). In some embodiments, the mouse has been irradiated. In some embodiments, a method of the disclosure comprises administering the human cells to the mouse. In some embodiments, a method further comprises irradiating the mouse prior to engrafting the human cells.

### Immunodeficient Mouse Deficient Expressing Human IL3, Human GM-CSF, Human SCF, and Human IL15 Transgenes

In some embodiments, the genome of an immunodeficient mouse model of the present disclosure comprises: an exogenous nucleic acid encoding huIL3; an exogenous nucleic acid encoding huGM-CS; and an exogenous nucleic acid encoding huSCF; and an exogenous nucleic acid encoding human huIL15 (e.g., a huIL15 transgene). In some embodiments, the genome of the mouse further comprises an endogenous Il2rg allele comprising a null mutation (Il2rg^{null}). In some embodiments, the genome of the mouse further comprises an endogenous Prkdc allele comprising a null mutation (Prkdc^{null}). In some embodiments, the genetic background of the mouse is NOD.Cg-Prkdc^{scid} Il2rg^{tm1Wjl}/SzJ.

In some embodiments, the immunodeficient mouse model has been engrafted with human cells selected from PBMCs, HSCs, and UCB cells (e.g., unfractionated human UCB cells). In some embodiments, the mouse has been irradiated. In some embodiments, a method of the disclosure comprises administering the human cells to the mouse. In some embodiments, a method further comprises irradiating the mouse prior to engrafting the human cells.

### Immunodeficient Mouse Deficient in MHC and Kit, and Expressing Human IL3, Human GM-CSF, Human SCF, Human IL7, and Human IL15 Transgenes

In some embodiments, the genome of an immunodeficient mouse model of the present disclosure comprises: an endogenous H2-K allele comprising a null mutation (H2-K^{null}); an endogenous H2-D allele comprising a null mutation (H2-D^{null}); an endogenous H2-A allele comprising a null mutation (H2-A^{null}); an endogenous Kit allele comprising a null mutation (Kit^{null}); an exogenous nucleic acid encoding huIL3; an exogenous nucleic acid encoding huGM-CS; an exogenous nucleic acid encoding huSCF; an exogenous nucleic acid encoding huIL7 (e.g., a huIL7 transgene); and an exogenous nucleic acid encoding human huIL15 (e.g., a huIL15 transgene). In some embodiments, the genome of the mouse further comprises an endogenous Il2rg allele comprising a null mutation (Il2rg^{null}). In some embodiments, the genome of the mouse further comprises an endogenous Prkdc allele comprising a null mutation (Prkdc^{null}). In some embodiments, the genetic background of the mouse is NOD.Cg-Prkdc^{scid} Il2rg^{tm1Wjl}/SzJ.

In some embodiments, the immunodeficient mouse model has been engrafted with human cells selected from PBMCs, HSCs, and UCB cells (e.g., unfractionated human UCB cells). In some embodiments, the mouse has been irradiated. In some embodiments, a method of the disclosure comprises administering the human cells to the mouse. In some embodiments, a method further comprises irradiating the mouse prior to engrafting the human cells.

In some embodiments, a humanized immunodeficient mouse model of the present disclosure comprises: an endogenous Il2rg allele comprising a null mutation (Il2rg^{null}); an endogenous H2-K allele comprising a null mutation (H2-K^{null}); an endogenous H2-D allele comprising a null mutation (H2-D^{null}); an endogenous H2-A allele comprising a null mutation (H2-A^{null}); an endogenous Kit allele comprising a null mutation (Kit^{null}); an exogenous nucleic acid encoding human interleukin-7 (huIL7); an exogenous nucleic acid encoding human interleukin-15 (huIL15); an exogenous nucleic acid encoding human interleukin-3 (huIL3); an exogenous nucleic acid encoding human granulocyte-macrophage colony-stimulating factor (huGM-CSF); and an exogenous nucleic acid encoding human Steel factor (huSCF), In some embodiments, the humanized immunodeficient mouse model further comprises endogenous Prkdc allele comprising a null mutation (Prkdc^{null}). In some embodiments, the humanized immunodeficient mouse model is homozygous for the Il2rg^{null} allele, is homozygous for the Prkdc^{null} allele, is homozygous for the H2-K^{null}allele, is homozygous for the H2-D^{null}allele, is homozygous for the H2-A^{null} allele, and is homozygous for the Kit^{null}allele. In some embodiments, the humanized immunodeficient mouse has been engrafted with HSCs. In some embodiments, the humanized immunodeficient mouse has been engrafted with PBMCs. In some embodiments, the humanized immunodeficient mouse has been engrafted with human umbilical cord blood cells (e.g., unfractionated human UCB cells).

In some embodiments, a humanized immunodeficient mouse model of the present disclosure comprises: an Il2rg^{tm1Wjl} allele; a Prkdc^{scid} allele; a H2-K1^{tm1Bpe} allele; a H2-D1^{tm1Bpe} allele; an H2-Ab1^{em1Mvw} allele; a Kit^{W-41J} allele; an exogenous nucleic acid encoding huIL7; an exogenous nucleic acid encoding huIL15, an exogenous nucleic acid encoding huIL3, an exogenous nucleic acid encoding huGM-CSF; and an exogenous nucleic acid encoding huSCF. In some embodiments, the humanized immunodeficient mouse model is homozygous for the Il2rg^{tm1Wjl} allele; is homozygous for the Prkdc^{scid} allele; is homozygous for the H2-K1^{tm1Bpe} allele; is homozygous for the H2-D1^{tm1Bpe} allele; is homozygous for the H2-Ab1^{em1Mvw} allele; and is homozygous for the Kit^{W-41J} allele. In some embodiments, the humanized immunodeficient mouse has been engrafted with HSCs. In some embodiments, the humanized immunodeficient mouse has been engrafted with PBMCs. In some embodiments, the humanized immunodeficient mouse has been engrafted with human umbilical cord blood cells (e.g., unfractionated human UCB cells).

In some embodiments, a humanized immunodeficient mouse model of the present disclosure comprises: an Il2rg^{tm1Sug} allele; a Prkdc^{scid} allele; a H2-K1^{tm1Bpe} allele; a H2-D1^{tm1Bpe} allele; an H2-Ab1^{em1Mvw} allele; a Kit^{W-41J} allele; an exogenous nucleic acid encoding huIL7; an exogenous nucleic acid encoding huIL15, an exogenous nucleic acid encoding huIL3, an exogenous nucleic acid encoding huGM-CSF; and an exogenous nucleic acid encoding huSCF. In some embodiments, the humanized immunodeficient mouse model is homozygous for the Il2rg^{tm1Sug} allele; is homozygous for the Prkdc^{scid} allele; is homozygous for the H2-K1^{tm1Bpe} allele; is homozygous for the H2-D1^{tm1Bpe} allele; is homozygous for the H2-Ab1^{em1Mvw} allele; and is homozygous for the Kit^{W-41J} allele. In some embodiments, the humanized immunodeficient mouse has been engrafted with HSCs. In some embodiments, the humanized immunodeficient mouse has been engrafted with PBMCs. In some embodiments, the humanized immunodeficient mouse has been engrafted with human umbilical cord blood cells.

### Immunodeficient Mouse Deficient in Kit, and Expressing Human IL3, Human GM-CSF, and Human SCF Transgenes

In some embodiments, the genome of an immunodeficient mouse model of the present disclosure comprises: an endogenous Kit allele comprising a null mutation (Kit^{null}); an exogenous nucleic acid encoding huIL3; an exogenous nucleic acid encoding huGM-CS; and an exogenous nucleic acid encoding huSCF. In some embodiments, the genome of the mouse further comprises an endogenous Il2rg allele comprising a null mutation (Il2rg^{null}). In some embodiments, the genome of the mouse further comprises an endogenous Prkdc allele comprising a null mutation (Prkdc^{null}). In some embodiments, the genetic background of the mouse is NOD.Cg-Prkdc^{scid} Il2rg^{tm1Wjl}/SzJ.

In some embodiments, the immunodeficient mouse model has been engrafted with human cells selected from PBMCs, HSCs, and UCB cells (e.g., unfractionated human UCB cells). In some embodiments, the mouse has been irradiated. In some embodiments, a method of the disclosure comprises administering the human cells to the mouse. In some embodiments, a method further comprises irradiating the mouse prior to engrafting the human cells.

### Human Cells

A “humanized” mouse is an immunodeficient mouse that has been engrafted with human cells. “Engraftment” refers to the process of the human cells migrating to, and incorporating into, an existing tissue of interest in vivo. Engrafted human cells repopulate an immunodeficient mouse with a functional human immune system. Most often, the human cells, such as hematopoietic stem cells (HSC) or peripheral blood mononuclear cells (PBMCs) are “fractionated” or “isolated”—separated from other cell types and subcellular components (e.g., by centrifugation)—prior to their use for humanization. Fractionation involves processing of a human cell population to enrich the population with one or more particular cell types. This processing may involve depleting the population of one or more specific cell types. Fractionated human HSC and PBMC populations develop into various human immune cell types. HSCs, through a process referred to a hematopoiesis, develop into dendritic cells, lymphoid cells, including T cells, natural killer cells, and B cells, and myeloid cells, including macrophages, granulocytes, platelets, and erythrocytes. PMBCs develop primarily into lymphocytes. In some embodiments, human HSCs are administered to an immunodeficient mouse model provided herein to humanize the mouse model. In some embodiments, human PBMCs are administered to an immunodeficient mouse model provided herein to humanize the mouse model.

Often, human HSCs and human PBMCs are obtained from human umbilical cord blood. Human umbilical cord blood is typically fractionated to separate HSCs and/or PBMCs from other cell types and subcellular components. Fractionation of the blood results in the removal of more mature immune cells, such as T cells, which contribute to the graft-versus-host response. Fractionation, however, requires added time, effort, and cost to preclinical evaluations, and can damage the cells. This can lower the survival and proliferation rate of human immune cell populations, thereby compromising successful engraftment of the immunodeficient mice.

Surprisingly, the inventors have found that with the immunodeficient mouse models provided herein, fractionation of human umbilical cord blood is not required. In some embodiments, unfractionated human umbilical cord blood may be administered to an immunodeficient mouse to facilitate engraftment of the human immune system. Because of the unique combination of alleles used to produce the immunodeficient mouse models provided herein, unfractionated human umbilical cord blood may be used without eliciting a graft-versus-host reaction in the mice, and in some embodiments, without conditioning the mice. “Unfractionated human umbilical cord blood” refers to human umbilical cord blood that has not been fractionated and therefore includes mature, HLA-restricted T cells.

Thus, in some embodiments, the immunodeficient mice are not ‘conditioned’ prior to engraftment of the human cells, e.g., prior to administration (e.g., injection) of the human cells. Conditioning refers to a group of treatments used to suppress the immune system and clear out stem cell niches prior to human cell engraftment. Conditioning typically includes myeloablative techniques, such as radiation and myeloablative chemotherapy (e.g., busulfan (1,4-butanediol dimethanesulfonate). Thus, in some embodiments, the humanized immunodeficient mouse models provided herein have not been conditioned. In some embodiments, method of producing the humanized immunodeficient mouse models do not include a conditioning step.

In some embodiments, the length of time an immunodeficient mouse provided herein supports engraftment of human immune cells (e.g., human T cells, human NK cells, and/or human myeloid cells) is extended by at least 25%, relative to a control mouse. For example, the length of time an immunodeficient mouse provided herein supports engraftment of human immune cells (e.g., human T cells, human NK cells, and/or human myeloid cells) may be extended by at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%, relative to a control mouse. In some embodiments, the length of time an immunodeficient mouse provided herein supports engraftment of human immune cells (e.g., human T cells, human NK cells, and/or human myeloid cells) is extended by 25%-100%, 25%-75%, 25%-50%, 50%-100%, 50%-75%, or 75%-100%, relative to a control mouse.

In some embodiments, the number of engrafted human immune cells (e.g., human T cells, human NK cells, and/or human myeloid cells) is increased in an immunodeficient mouse provided herein by at least 25%, relative to a control mouse. For example, the number of engrafted human immune cells (e.g., human T cells, human NK cells, and/or human myeloid cells) may be increased in an immunodeficient mouse provided herein by at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%, relative to a control mouse. In some embodiments, the number of engrafted human immune cells (e.g., human T cells, human NK cells, and/or human myeloid cells) is increased in an immunodeficient mouse provided herein by 25%-100%, 25%-75%, 25%-50%, 50%-100%, 50%-75%, or 75%-100%, relative to a control mouse.

In some embodiments, the rate of cell death of engrafted human immune cells (e.g., human T cells, human NK cells, and/or human myeloid cells) in an immunodeficient mouse provided herein is decreased by at least 25%. For example, the rate of cell death of engrafted human immune cells (e.g., human T cells, human NK cells, and/or human myeloid cells) in an immunodeficient mouse provided herein is decreased by at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%, relative to a control mouse. In some embodiments, the rate of cell death of engrafted human immune cells (e.g., human T cells, human NK cells, and/or human myeloid cells) in an immunodeficient mouse provided herein is decreased by 25%-100%, 25%-75%, 25%-50%, 50%-100%, 50%-75%, or 75%-100%, relative to a control mouse.

In some embodiments, the rate of cell proliferation of engrafted human immune cells (e.g., human T cells, human NK cells, and/or human myeloid cells) in an immunodeficient mouse provided herein is increased by at least 25%. For example, the rate of cell proliferation of engrafted human immune cells (e.g., human T cells, human NK cells, and/or human myeloid cells) in an immunodeficient mouse provided herein may be increased by at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%, relative to a control mouse. In some embodiments, the rate of cell proliferation of engrafted human immune cells (e.g., human T cells, human NK cells, and/or human myeloid cells) in an immunodeficient mouse provided herein is increased by 25%-100%, 25%-75%, 25%-50%, 50%-100%, 50%-75%, or 75%-100%, relative to a control mouse.

A control mouse may be, for example, a mouse that is no immunodeficient and/or is not humanized. In some embodiments, a control mouse is a NOD scid gamma mouse (NSG® mouse).

Methods of injecting immunodeficient mice with human cells to yield a humanized mouse model include but are not limited to intraperitoneal or intravenous injection (Shultz et al., J Immunol, 2015, 174:6477-6489; Pearson et al., Curr Protoc Immunol. 2008; 15-21; Kim et al., AIDS Res Hum Retrovirus, 2016, 32(2): 194-2020; Yaguchi et al., Cell & Mol Immunol, 2018, 15:953-962). In some embodiments, the mouse is injected in the facial vein, the heart, or the liver.

In some embodiments, the mouse is injected with about 1×10⁴ to about 1×10¹⁰ human cells. For example, the mouse may be injected with about 1×10⁴ to about 1×10⁹, about 1×10⁴ to about 1×10⁸, about 1×10⁴ to about 1×10⁷, about 1×10⁴ to about 1×10⁶, about 1×10⁴ to about 1×10⁵, about 1×10⁵ to about 1×10¹⁰, about 1×10⁵ to about 1×10⁹, about 1×10⁵ to about 1×10⁸, about 1×10⁵ to about 1×10⁷, about 1×10⁵ to about 1×10⁶, about 1×10⁶ to about 1×10¹⁰, about 1×10⁶ to about 1×10⁹, about 1×10⁶ to about 1×10⁸, or about 1×10⁶ to about 1×10⁷ human cells. In some embodiments, the mouse is injected with about 1×10⁴, about 1×10⁵, about 1×10⁶, about 1×10⁷, about 1×10⁸, about 1×10⁹, about 1×10¹⁰ human cells.

In some embodiments, the human cells are formulated in a solution (e.g., a buffered solution). In some embodiments, the solution has a volume of about 50 μl to about 250 μl. For example, the solution may have a volume of about 50 μl, 60 μl, 70 μl, 80 μl, 90 μl, 100 μl, 110 μl, 120 μl, 130 μl, 140 μl, 150 μl, 160 μl, 170 μl, 180 μl, 190 μl, 200 μl, 210 μl, 220 μl, 230 μl, 240 μl, or 250 μl.

In some embodiments, the mouse is injected with about 50 μl to about 250 μl of human umbilical cord blood. For example, the solution may have a volume of about 50 μl, 60 μl, 70 μl, 80 μl, 90 μl, 100 μl, 110 μl, 120 μl, 130 μl, 140 μl, 150 μl, 160 μl, 170 μl, 180 μl, 190 μl, 200 μl, 210 μl, 220 μl, 230 μl, 240 μl, or 250 μl of human umbilical cord blood.

### Nucleic Acids: Engineering and Delivery

The nucleic acids provided herein, in some embodiments, are engineered (e.g., exogenous). An engineered nucleic acid is a nucleic acid (e.g., at least two nucleotides covalently linked together, and in some instances, containing phosphodiester bonds, referred to as a phosphodiester backbone) that does not occur in nature. Engineered nucleic acids include recombinant nucleic acids and synthetic nucleic acids. A recombinant nucleic acid is a molecule that is constructed by joining nucleic acids (e.g., isolated nucleic acids, synthetic nucleic acids or a combination thereof) from two different organisms (e.g., human and mouse). A synthetic nucleic acid is a molecule that is amplified or chemically, or by other means, synthesized. A synthetic nucleic acid includes those that are chemically modified, or otherwise modified, but can base pair with (bind to) naturally occurring nucleic acid molecules. Recombinant and synthetic nucleic acids also include those molecules that result from the replication of either of the foregoing.

An engineered nucleic acid may comprise DNA (e.g., genomic DNA, cDNA or a combination of genomic DNA and cDNA), RNA or a hybrid molecule, for example, where the nucleic acid contains any combination of deoxyribonucleotides and ribonucleotides (e.g., artificial or natural), and any combination of two or more bases, including uracil, adenine, thymine, cytosine, guanine, inosine, xanthine, hypoxanthine, isocytosine and isoguanine.

In some embodiments, a nucleic acid is a complementary DNA (cDNA). cDNA is synthesized from a single-stranded RNA (e.g., messenger RNA (mRNA) or microRNA (miRNA)) template in a reaction catalyzed by reverse transcriptase.

Engineered (e.g., exogenous) nucleic acids of the present disclosure may be produced using standard molecular biology methods (see, e.g., Green and Sambrook, Molecular Cloning, A Laboratory Manual, 2012, Cold Spring Harbor Press). In some embodiments, nucleic acids are produced using GIBSON ASSEMBLY® Cloning (see, e.g., Gibson, D. G. et al. Nature Methods, 343-345, 2009; and Gibson, D. G. et al. Nature Methods, 901-903, 2010, each of which is incorporated by reference herein). GIBSON ASSEMBLY® typically uses three enzymatic activities in a single-tube reaction: 5′ exonuclease, the 3′ extension activity of a DNA polymerase and DNA ligase activity. The 5′ exonuclease activity chews back the 5′ end sequences and exposes the complementary sequence for annealing. The polymerase activity then fills in the gaps on the annealed domains. A DNA ligase then seals the nick and covalently links the DNA fragments together. The overlapping sequence of adjoining fragments is much longer than those used in Golden Gate Assembly, and therefore results in a higher percentage of correct assemblies. Other methods of producing engineered nucleic acids may be used in accordance with the present disclosure.

A gene is a distinct sequence of nucleotides, the order of which determines the order of monomers in a polynucleotide or polypeptide. A gene typically encodes a protein. A gene may be endogenous (occurring naturally in a host organism) or exogenous (transferred, naturally or through genetic engineering, to a host organism). An allele is one of two or more alternative forms of a gene that arise by mutation and are found at the same locus on a chromosome. A gene, in some embodiments, includes a promoter sequence, coding regions (e.g., exons), non-coding regions (e.g., introns), and regulatory regions (also referred to as regulatory sequences).

A mouse comprising a human gene is considered to comprise a human transgene. A transgene is a gene exogenous to a host organism. That is, a transgene is a gene that has been transferred, naturally or through genetic engineering, to a host organism. A transgene does not occur naturally in the host organism (the organism, e.g., mouse, comprising the transgene).

A promoter is a nucleotide sequence to which RNA polymerase binds to initial transcription (e.g., ATG). Promoters are typically located directly upstream from (at the 5′ end of) a transcription initiation site. In some embodiments, a promoter is an endogenous promoter. An endogenous promoter is a promoter that naturally occurs in that host animal.

An open reading frame is a continuous stretch of codons that begins with a start codon (e.g., ATG), ends with a stop codon (e.g., TAA, TAG, or TGA), and encodes a polypeptide, for example, a protein. An open reading frame is operably linked to a promoter if that promoter regulates transcription of the open reading frame.

An exon is a region of a gene that codes for amino acids. An intron (and other non-coding DNA) is a region of a gene that does not code for amino acids.

A nucleotide sequence encoding a product (e.g., protein), in some embodiments, has a length of 200 base pairs (bp) to 100 kilobases (kb). The nucleotide sequence, in some embodiments, has a length of at least 10 kb. For example, the nucleotide sequence may have a length of at least 15 kb, at least 20 kb, at least 25 kb, at least 30 kb, or at least 35 kb. In some embodiments, the nucleotide sequence has a length of 10 to 100 kb, 10 to 75 kb, 10 to 50 kb, 10 to 30 kb, 20 to 100 kb, 20 to 75 kb, 20 to 50 kb, 20 to 30 kb, 30 to 100 kb, 30 to 75 kb, or 30 to 50 kb.

Any one of the nucleic acids provided herein may have a length of 200 bp to 500 kb, 200 bp to 250 kb, or 200 bp to 100 kb. A nucleic acid, in some embodiments, has a length of at least 10 kb. For example, a nucleic acid may have a length of at least 15 kb, at least 20 kb, at least 25 kb, at least 30 kb, at least 35 kb, at least 50 kb, at least 100 kb, at least 200 kb, at least 300 kb, at least 400 kb, or at least 500 kb. In some embodiments, a nucleic acid has a length of 10 to 500 kb, 20 to 400 kb, 10 to 300 kb, 10 to 200 kb, or 10 to 100 kb. In some embodiments, a nucleic acid has a length of 10 to 100 kb, 10 to 75 kb, 10 to 50 kb, 10 to 30 kb, 20 to 100 kb, 20 to 75 kb, 20 to 50 kb, 20 to 30 kb, 30 to 100 kb, 30 to 75 kb, or 30 to 50 kb. A nucleic acid may be circular or linear.

The nucleic acids described herein, in some embodiments, include a modification. A modification, with respect to a nucleic acid, is any manipulation of the nucleic acid, relative to the corresponding wild-type nucleic acid (e.g., the naturally occurring nucleic acid). A genomic modification is thus any manipulation of a nucleic acid in a genome (e.g., in a coding region, non-coding region, and/or regulatory region), relative to the corresponding wild-type nucleic acid (e.g., the naturally occurring (unmodified) nucleic acid) in the genome. Non-limiting examples of nucleic acid (e.g., genomic) modifications include deletions, insertions, “indels” (deletion and insertion), and substitutions (e.g., point mutations). In some embodiments, a deletion, insertion, indel, or other modification in a gene results in a frameshift mutation such that the gene no longer encodes a functional product (e.g., protein). Modifications also include chemical modifications, for example, chemical modifications of at least one nucleobase. Methods of nucleic acid modification, for example, those that result in gene inactivation, are known and include, without limitation, RNA interference, chemical modification, and gene editing (e.g., using recombinases or other programmable nuclease systems, e.g., CRISPR/Cas, TALENs, and/or ZFNs).

A null mutation, as is known in the art, results in a gene product with little or no function. A null mutation results in a gene product with no detectable/measurable function.

A nucleic acid, such as an allele or alleles of a gene, may be modified such that it does not produce a detectable level of a functional gene product (e.g., a functional protein). Thus, null allele is an allele that does not produce a detectable level of a functional gene product (e.g., a functional protein). A detectable level of a protein is any level of protein detected using a standard protein detection assay, such as flow cytometry and/or an ELISA. In some embodiments, a null allele is not transcribed. In some embodiments, a null allele does not encode a functional protein.

Vectors used for delivery of a nucleic acid include minicircles, plasmids, bacterial artificial chromosomes (BACs), and yeast artificial chromosomes. It should be understood, however, that a vector may not be needed. For example, a circularized or linearized nucleic acid may be delivered to an embryo without its vector backbone. Vector backbones are small (˜4 kb), while donor DNA to be circularized can range from >100 bp to 50 kb, for example.

Methods for delivering nucleic acids to mouse embryos (e.g., mouse) for the production of transgenic mice include, but are not limited to, electroporation (see, e.g., Wang W et al. J Genet Genomics 2016; 43(5):319-27; WO 2016/054032; and WO 2017/124086, each of which is incorporated herein by reference), DNA microinjection (see, e.g., Gordon and Ruddle, Science 1981: 214: 1244-124, incorporated herein by reference), embryonic stem cell-mediated gene transfer (see, e.g., Gossler et al., Proc. Natl. Acad. Sci. 1986; 83: 9065-9069, incorporated herein by reference), and retrovirus-mediated gene transfer (see, e.g., Jaenisch, Proc. Natl. Acad. Sci. 1976; 73: 1260-1264, incorporated herein by reference), any of which may be used as provided herein.

### Genomic Editing Methods

The present application contemplates the use of a variety of gene editing technologies using engineered nucleic acids, for example, to knockout a target endogenous gene (e.g., Il2rg, Prkdc, H2-K, H2-D, H2-A, and/or Kit) or to introduce nucleic acids into the genome of a mouse (e.g., to produce a transgenic mouse expressing human IL7, human IL15, human IL3, human GM-CSF, and/or human SCF). An immunodeficient mouse described herein may be produced by any gene editing technology known in the art.

Engineered nucleic acids, such as guide RNAs, donor polynucleotides, and other nucleic acid coding sequences, for example, may be introduced to a genome of an embryo or cell (e.g., stem cell) using any suitable method. The present application contemplates the use of a variety of gene editing technologies, for example, to delete nucleic acids from the genome of an embryo or cell to produce a knockout mouse or to introduce nucleic acids into the genome of an embryo or cell to produce a transgenic mouse. Non-limiting examples include programmable nuclease-based systems, such as clustered regularly interspaced short palindromic repeat (CRISPR) systems, zinc-finger nucleases (ZFNs), and transcription activator-like effector nucleases (TALENs). See, e.g., Carroll D Genetics. 2011; 188(4): 773-782; Joung J K et al. Nat Rev Mol Cell Biol. 2013; 14(1): 49-55; and Gaj T et al. Trends Biotechnol. 2013 July; 31(7): 397-405, each of which is incorporated by reference herein.

In some embodiments, a CRISPR system is used to edit the genome of mouse (e.g., mouse) embryos provided herein. See, e.g., Harms D W et al., Curr Protoc Hum Genet. 2014; 83: 15.7.1-15.7.27; and Inui M et al., Sci Rep. 2014; 4: 5396, each of which are incorporated by reference herein). For example, Cas9 mRNA or protein, one or multiple guide RNAs (gRNAs), and/or a donor nucleic acid can be delivered, e.g., injected or electroporated, directly into mouse embryos at the one-cell (zygote) stage or a later stage to facilitate homology directed repair (HDR), for example, to delete a nucleic acid sequence from the genome or to introduce an engineered nucleic acid (e.g., donor nucleic acid) into the genome.

The CRISPR/Cas system is a naturally occurring defense mechanism in prokaryotes that has been repurposed as an RNA-guided-DNA-targeting platform for gene editing. Engineered CRISPR systems contain two main components: a guide RNA (gRNA) and a CRISPR-associated endonuclease (e.g., Cas protein). The gRNA is a short synthetic RNA composed of a scaffold sequence for nuclease-binding and a user-defined nucleotide spacer (e.g., ˜15-25 nucleotides, or ˜20 nucleotides) that defines the genomic target (e.g., gene) to be modified. Thus, one can change the genomic target of the Cas protein by simply changing the target sequence present in the gRNA. In some embodiments, the Cas9 endonuclease is from Streptococcus pyogenes (NGG PAM) or Staphylococcus aureus (NNGRRT or NNGRR(N) PAM), although other Cas9 homologs, orthologs, and/or variants (e.g., evolved versions of Cas9) may be used, as provided herein. Additional non-limiting examples of RNA-guided nucleases that may be used as provided herein include Cpf1 (TTN PAM); SpCas9 D1135E variant (NGG (reduced NAG binding) PAM); SpCas9 VRER variant (NGCG PAM); SpCas9 EQR variant (NGAG PAM); SpCas9 VQR variant (NGAN or NGNG PAM); Neisseria meningitidis (NM) Cas9 (NNNNGATT PAM); Streptococcus thermophilus (ST) Cas9 (NNAGAAW PAM); and Treponema denticola (TD) Cas9 (NAAAAC). In some embodiments, the CRISPR-associated endonuclease is selected from Cas9, Cpf1, C2c1, and C2c3. In some embodiments, the Cas nuclease is Cas9.

A guide RNA comprises at least a spacer sequence that hybridizes to (binds to) a target nucleic acid sequence and a CRISPR repeat sequence that binds the endonuclease and guides the endonuclease to the target nucleic acid sequence. As is understood by the person of ordinary skill in the art, each gRNA is designed to include a spacer sequence complementary to its genomic target sequence. See, e.g., Jinek et al., Science, 2012; 337: 816-821 and Deltcheva et al., Nature, 2011; 471: 602-607, each of which is incorporated by reference herein.

In some embodiments, the RNA-guided nuclease and the gRNA are complexed to form a ribonucleoprotein (RNP), prior to delivery to an embryo.

The concentration of RNA-guided nuclease or nucleic acid encoding the RNA-guided nuclease may vary. In some embodiments, the concentration is 100 ng/μl to 1000 ng/μl. For example, the concentration may be 100, 150, 200, 250, 300, 350, 400, 450, 500, 550, 600, 650, 700, 750, 800, 850, 900, 950, or 1000 ng/μl. In some embodiments, the concentration is 100 ng/μl to 500 ng/μl, or 200 ng/μl to 500 ng/μl.

The concentration of gRNA may also vary. In some embodiments, the concentration is 200 ng/μl to 2000 ng/μl. For example, the concentration may be 200, 300, 400, 500, 600, 700, 800, 900, 1000, 1100, 1200, 1300, 1400, 1500, 1600, 1700, 1700, 1900, or 2000 ng/μl. In some embodiments, the concentration is 500 ng/μl to 1000 ng/μl. In some embodiments, the concentration is 100 ng/μl to 1000 ng/μl. For example, the concentration may be 100, 150, 200, 250, 300, 350, 400, 450, 500, 550, 600, 650, 700, 750, 800, 850, 900, 950, or 1000 ng/μl.

In some embodiments, the ratio of concentration of RNA-guided nuclease or nucleic acid encoding the RNA-guided nuclease to the concentration of gRNA is 2:1. In other embodiments, the ratio of concentration of RNA-guided nuclease or nucleic acid encoding the RNA-guided nuclease to the concentration of gRNA is 1:1.

A donor nucleic acid typically includes a sequence of interest flanked by homology arms.

Homology arms are regions of the ssDNA that are homologous to regions of genomic DNA located in a genomic locus. One homology arm is located to the left (5′) of a genomic region of interest (into which a sequence of interest is introduced) (the left homology arm) and another homology arm is located to the right (3′) of the genomic region of interest (the right homology arm). These homology arms enable homologous recombination between the ssDNA donor and the genomic locus, resulting in insertion of the sequence of interest into the genomic locus of interest (e.g., via CRISPR/Cas9-mediated homology directed repair (HDR)).

The homology arms may vary in length. For example, each homology arm (the left arm and the right homology arm) may have a length of 20 nucleotide bases to 1000 nucleotide bases. In some embodiments, each homology arm has a length of 20 to 200, 20 to 300, 20 to 400, 20 to 500, 20 to 600, 20 to 700, 20 to 800, or 20 to 900 nucleotide bases. In some embodiments, each homology arm has a length of 20, 30, 40, 50, 60, 70, 80, 90, 100, 150, 200, 250, 300, 350, 400, 450, 500, 550, 600, 650, 700, 750, 800, 850, 900, 950, or 1000 nucleotide bases. In some embodiments, the length of one homology arm differs from the length of the other homology arm. For example, one homology arm may have a length of 20 nucleotide bases, and the other homology arm may have a length of 50 nucleotide bases. In some embodiments, the donor DNA is single stranded. In some embodiments, the donor DNA is double stranded. In some embodiments, the donor DNA is modified, e.g., via phosphorothioation. Other modifications may be made.

### Methods of Use

A critical roadblock for the translation of human cell therapy to the clinic is the need for robust preclinical animal models for evaluation of the efficacy, safety and importantly, immunogenicity of human cellular therapy (e.g., such as human stem cell-based and immune cell-based therapy). Because the cell therapy products are of human origin, it has been challenging to test these products in vivo using other species due to potent xenograft responses. One approach to evaluating cell therapy products without putting patients at risk is to use humanized mice—immunodeficient mice engrafted with functional human cells, tissues, and immune system. Humanized mice can provide a critically needed preclinical bridge for evaluation of the safety, efficacy, and immunogenicity of human stem cell derived cell products.

Thus, the humanized immunodeficient mouse models of the present disclosure may be used, in some embodiments, to evaluate the clinical efficacy of cell therapy products. Non-limiting examples of cell therapy products include cellular immunotherapies, cancer vaccines, and other types of both autologous and allogeneic cells for certain therapeutic indications, including hematopoietic stem cells and adult and embryonic stem cells. Cell therapy refers to the transfer of autologous or allogeneic cellular material into a patient for medical purposes. The mouse models provided herein, which aim to replicate the human immune system may be used to evaluate such cell therapies.

Thus, in some embodiments, a method provided herein comprises administering a cell therapy product to a humanized immunodeficient mouse described herein.

In some embodiments, the humanized immunodeficient mouse has been engrafted with HSCs. In some embodiments, the humanized immunodeficient mouse has been engrafted with PBMCs. In some embodiments, the humanized immunodeficient mouse has been engrafted with human umbilical cord blood cells.

The methods may further comprise assessing one or more clinically relevant characteristics of the cell therapy product or one or more clinically relevant effect on the engrafted human immune system. Clinically relevant characteristics may include, for example, a slowing of tumor growth or resolution of infection.

In some embodiments, the cell therapy product is designed to treat (e.g., alleviate one or more symptom of) a cancer. Non-limiting examples of human cancers include: adenoid cystic carcinoma, adrenal gland tumor, amyloidosis, anal cancer, appendix cancer, astrocytoma, ataxia-telegiectasia, Beckwith-Wiedeman syndrome, bile duct cancer, Birt-Hogg-Dube syndrome, bladder cancer, bone cancer, brain stem glioma, brain cancer, breast cancer, Carney complex, cervical cancer, colorectal cancer, Cowden Syndrome, craniopharyngioma, desmoid tumor, desmoplastic infantile ganglioglioma, ependymoma, esophageal cancer, Ewing sarcoma, eye cancer, eyelid cancer, familial adenomatous polyposis, familial GIST, familial malignant melanoma, familial pancreatic cancer, gallbladder cancer, gastrointestinal stromal tumor (GIST), germ cell tumor, gestational trophoblastic disease, head and neck cancer, hereditary breast and ovarian cancer, hereditary diffuse gastric cancer, hereditary leiomyomatosis and renal cell cancer, hereditary mixed polyposis syndrome, hereditary pancreatitis, hereditary papillary renal carcinoma, HIV/AIDS-related cancer, juvenile polyposis syndrome, kidney cancer, lacrimal gland tumor, laryngeal and hypopharyngeal cancer, leukemia, Li-Fraumeni syndrome, liver cancer, lung cancer, lymphoma, Lynch syndrome, mastocytosis, medullablastoma, melanoma, meningioma, mesothelioma, multiple endocrine neoplasia type 1, multiple endocrine neoplasia type 2, multiple myeloma, MUTYH-associated polyposis, myelodysplastic syndrome, nasal cavity and paranasal sinus cancer, nasopharyngeal cancer, neuroblastoma, neuroendocrine tumor of the gastrointestinal tract, neuroendocrine tumor of the lung, neuroendocrine tumor of the pancreas, neurofibromatosis type 1, neurofibromatosis type 2, nevoid basal cell carcinoma syndrome, oral and oropharyngeal cancer, osteosarcoma, ovarian cancer, pancreatic cancer, parathyroid cancer, penile cancer, Peutz-Jeghers syndrome, pheochromocytoma and paraganglioma, pituitary gland tumor, pleuropulmonary blastoma, prostate cancer, retinoblastoma, rhabdomyosarcoma, salivary gland cancer, sarcoma, non-melanoma skin cancer, small bowel cancer, stomach cancer, testicular cancer, thymoma and thymic carcinoma, thyroid cancer, tuberous sclerosis complex, uterine cancer, vaginal cancer, Von Hippel-Lindau syndrome, vulvar cancer, Waldenstrom Macroglobulinemia, Werner syndrome, Wilms tumor, and xeroderma pigmentosum.

In some embodiments, the cell therapy product is designed to treat an autoimmune disease.

In some embodiments, the cell therapy product is designed to treat a genetic disorder.

There are several types of cell therapy products that may be tested using the humanized immunodeficient mouse models of the present disclosure. In some embodiments, the cell therapy product is a stem cell-based cell therapy product. In some embodiments, the cell therapy product is a non-stem cell-based cell therapy product. In some embodiments, the cell therapy product is an adoptive cell therapy (ACT) product. In some embodiments, the cell therapy product is a scaffold-based or scaffold-free cell therapy product. In some embodiments, the cell therapy product is a bone marrow aspirate (BMA)-derived cell therapy product. The present disclosure also contemplates the testing of acellular therapies, for example, with multicellular components. See, e.g., El-Hakim El-Kadiry A et la., Front. Med., 22 Nov. 2021 Sec. Gene and Cell Therapy, incorporated herein by reference.

A humanized immunodeficient mouse model of the present disclosure may be used, in some embodiments, to study a disease associated with the human immune system. A disease associated with the human immune system is a disease or disorder with a pathophysiology that is, at least in part, attributed to or affected by human immune cells. Non-limiting examples of diseases associated with the human immune system that may be studied with the humanized immunodeficient mouse models provided herein include: muscle disorders (e.g., muscular dystrophy, myopathy, etc.), autoimmune disorders (e.g., rheumatoid arthritis, Crohn's disease, ulcerative colitis, lupus, or any other autoimmune disease or disorder provided herein), cancers (e.g., melanoma, leukemia, lymphoma, or any other cancers provided herein), metabolic disorders (e.g., diabetes, obesity, etc). Studying a disease associated with the human immune system may comprise, in some embodiments, administering a potential treatment for the disease to a humanized immunodeficient mouse model and characterizing the effect of the potential treatment. A potential treatment may be a cell therapy product, a protein (e.g., antibody, peptide, etc.), a small molecule, or any other potential treatment known in the art. Characterizing the effect of the potential treatment may be, for example, measuring the level of proteins (e.g., biomarkers) associated with the disease in a sample from the mouse, imaging the mouse to study disease progression, measuring survival after the potential treatment, or any other method of characterizing a disease known in the art.

In some embodiments, a humanized immunodeficient mouse model of the present disclosure may be used to study a muscular dystrophy. Muscular dystrophy is an inherited disease characterized by the weakening and wasting away of muscle tissue, with or without nerve involvement. Non-limiting examples of muscular dystrophy include: Becker, congenital, Duchenne, distal, Emery-Dreifuss, facioscapulohumeral (FSHD), limb-girdle, myotonic, and oculopharyngeal. In some embodiments, a humanized immunodeficient mouse model of the present disclosure may be used to study FSHD muscular dystrophy.

### Therapeutic Modalities

Therapeutic modalities are the different approaches and strategies used in the treatment of various diseases and health conditions in a subject. Herein, the terms “subject,” “patient,” and “individual” are used interchangeably. In some embodiments, a subject is a human subject. Other animal subjects are also contemplated herein. Some of the most common therapeutic modalities include pharmacotherapy, which involves the use of drugs to treat diseases and manage symptoms. Other therapeutic modalities include gene therapy and immunotherapy, which use genetic manipulation and the immune system, respectively, to treat diseases such as cancer and genetic disorders. There are many therapeutic modalities available, and the choice of treatment depends on the patient's condition, medical history, and the expertise of the healthcare provider.

The therapeutic modality, in some embodiments, is a targeted therapeutic. Targeted therapy is a type of treatment uses drugs or other substances to identify and attack cells more precisely than standard therapies. Unlike chemotherapy, for example, which can affect healthy cells as well as cancer cells, targeted therapy is designed to interfere with specific molecules or pathways involved in cancer cell growth and survival. Targeted therapy is based on the principle that diseased cells often have certain genetic or molecular abnormalities that distinguish them from normal cells. By targeting these specific abnormalities, targeted therapies can be more effective and less toxic than traditional therapies, such as chemotherapy. Non-limiting examples of targeted therapies include drugs that block the activity of specific enzymes or growth factor receptors, as well as immunotherapies that stimulate the immune system to recognize and attack diseased cells.

In some embodiments, the mouse models provided herein are used to test effects of a therapeutic modality. Non-limiting examples of therapeutic modalities that may be used as provided herein include antibodies, small molecule drugs, gene therapies, cell therapies, vaccines, hormones, enzyme replacement therapies, and nucleic acid-based therapies.

Antibodies are proteins produced by the immune system that can specifically recognize and bind to foreign substances, such as viruses and bacteria, and help neutralize or eliminate them from the body. Antibodies can also be designed and produced in the laboratory and used as therapeutics to target specific proteins or cells in the body. A therapeutic antibody used herein may be a full-length antibody or an antibody fragment. Antibody fragments are smaller fragments of a full-length antibody that have antigen-binding capacity. Some of the most commonly used antibody fragments include Fab (fragment antigen-binding) fragments, F(ab′)2 (fragment antigen-binding dimer) fragments, single-chain variable fragment (scFv), nanobodies, bispecific antibodies, diabodies, triabodies, and domain antibodies (dAbs). Fab fragments are the variable regions of the antibody that contain the antigen-binding site. Fab fragments can be produced by enzymatic cleavage of the antibody molecule and are often used in diagnostic applications, for example. F(ab′)2 fragments are the Fab fragments joined together by a disulfide bond, resulting in a fragment that can bind two antigen molecules simultaneously. Single-chain variable fragments are recombinant antibody fragments that include the variable regions of the heavy and light chains of an antibody connected by a short linker peptide. Single-chain variable fragments can be produced in bacteria or yeast and are often used for targeting tumors or other disease-related antigens, for example. Nanobodies are single-domain antibody fragments derived from camelid or shark antibodies that have a small size and high stability. Nanobodies can be produced by genetic engineering, for example. Bispecific antibodies are antibodies that can bind to two different antigens simultaneously. Bispecific antibodies can be produced by fusing two different Fab or scFv fragments together or by engineering a single antibody molecule to contain two different antigen-binding sites. Diabodies are artificially engineered antibodies consisting of two different single-chain variable fragments (scFv) joined together. Diabodies have a small size and can bind to two different antigens simultaneously. Triabodies are artificially engineered antibodies consisting of three different single-chain variable fragments (scFv) joined together. Triabodies have a small size and can bind to three different antigens simultaneously. Domain antibodies are antibody fragments consisting of a single variable domain of the antibody that can be produced in bacteria or yeast. dAbs have a small size and high stability.

Small molecule drugs are low molecular weight (e.g., less than 10 kDa) compounds that can bind to and modify the activity of specific proteins in the body. Small molecule drugs are often used to treat diseases such as cancer, hypertension, and diabetes, for example.

Gene therapies involve the delivery of genetic material, such as DNA or RNA, to cells in the body to correct genetic defects or modify cellular function, for example.

Cell therapies involve the transplantation or modification of cells in the body to replace damaged or diseased cells or tissues, for example. Non-limiting examples of cell therapies include stem cell therapy, CAR T-cell therapy, gene editing using CRISPR/Cas9, mesenchymal stem cell therapy, retinal pigment epithelial cell therapy, natural killer cell therapy, tumor-infiltrating lymphocyte therapy, dendritic cell therapy, cord blood stem cell therapy, and tissue engineering.

Vaccines are biological preparations that stimulate the immune system to produce a protective immune response against a specific infectious agent, such as a virus or bacteria. There are several types of vaccines, each of which uses a different method to stimulate the immune response. Some of the most common types of vaccines include inactivated vaccines, live attenuated vaccines, subunit, recombinant, and conjugate vaccines, mRNA vaccines, viral vector vaccines, and DNA vaccines.

Hormones are chemical messengers produced by the endocrine system that regulate various physiological functions in the body. Hormones can be used as therapeutics to treat a variety if conditions such as diabetes, thyroid disorders, and growth hormone deficiency.

Enzyme replacement therapies involve the administration of enzymes to replace or supplement enzymes that are deficient or missing in the body.

Nucleic acid-based therapies involve the delivery of nucleic acids, such as DNA or RNA, to cells in the body to modify gene expression or cellular function. Nucleic acid-based therapies include antisense oligonucleotide therapy and RNA interference therapy. RNA interference (RNAi) therapy is a type of gene therapy that involves the use of small RNA molecules to silence or “knock down” the expression of specific genes in the body. Examples of types of RNAi molecules include short interfering RNA (siRNA), microRNA (miRNA), short hairpin RNA (shRNA), ribozymes, aptamers, antisense RNA, and CRISPR RNA (crRNA). Short interfering RNA is a double-stranded RNA molecule that is typically 21-23 nucleotides in length. siRNA molecules are used to silence specific genes by targeting their mRNA for degradation. MicroRNAs are small, single-stranded RNA molecules that are typically 20-24 nucleotides in length. miRNA molecules are involved in regulating the expression of multiple genes by targeting their mRNAs for degradation or translation inhibition. Short hairpin RNA is a single-stranded RNA molecule that is typically 19-29 nucleotides in length and folds back on itself to form a hairpin structure. shRNA molecules are used to silence specific genes by targeting their mRNA for degradation. Ribozymes are RNA molecules that have enzymatic activity and can cleave specific RNA molecules, including mRNA. Aptamers are RNA molecules that can bind to specific targets, such as proteins or other molecules, with high affinity and specificity. Antisense RNA is a single-stranded RNA molecule that is complementary to a specific mRNA molecule. Antisense RNA molecules are used to inhibit translation of the target mRNA by forming a double-stranded RNA molecule that is degraded by the cell. CRISPR RNA is a RNA molecule that is part of the CRISPR-Cas9 system, a genome editing tool that can be used to target specific genes for deletion or modification.

### Routes of Administration

Cells (e.g., human cells) and/or therapeutic modalities (or other substances/agents) of the present disclosure may be administered to an immunodeficient mouse via systemic administration or via local administration, for example.

In some embodiments, a cell or therapeutic modality is administered via systemic administration. Systemic routes of administration in mice involve the methods by which drugs or therapeutic modalities are introduced into the body of mice to achieve systemic distribution and desired effects. Intravenous (IV) injection is a commonly used route in mice, involving the direct delivery of drugs or therapeutic modalities into veins such as the tail vein, lateral tail vein, retro-orbital sinus, or jugular vein. IV injection provides rapid and direct access to the systemic circulation, ensuring immediate distribution throughout the body. This route is suitable for substances requiring quick systemic effects. Intraperitoneal (IP) injection involves the delivery of drugs or therapeutic modalities into the peritoneal cavity of mice. The substance is absorbed through the peritoneal membrane and enters the systemic circulation. This route provides widespread distribution of the drug within the abdominal cavity and systemic circulation, making it suitable for drugs requiring extensive contact with abdominal organs. Subcutaneous (SC) injection involves the delivery of drugs or therapeutic modalities into the subcutaneous tissue, typically in the dorsal region or behind the neck of mice. The substance is absorbed into the systemic circulation through the capillaries in the subcutaneous tissue. SC injection provides slower but sustained release of the drug into the systemic circulation, making it suitable for substances requiring a longer duration of action. Intramuscular (IM) injection entails the delivery of drugs or therapeutic modalities directly into the muscle tissue of mice, such as the quadriceps or gastrocnemius muscle. The substance is absorbed through the capillaries within the muscle and enters the systemic circulation. IM injection allows for sustained release and longer duration of action compared to other routes, making it suitable for substances requiring a sustained effect. Oral gavage involves the administration of drugs or therapeutic modalities directly into the stomach of mice using a feeding needle or oral gavage needle. This route is commonly used for substances that are orally bioavailable and stable in the gastrointestinal tract. Oral gavage allows for systemic distribution through absorption in the gastrointestinal tract, making it suitable for substances that can be administered orally. Inhalation involves the administration of drugs or therapeutic modalities through inhalation of aerosolized substances. Inhalation chambers or specialized devices are used to deliver the substance to the respiratory system of mice. Inhalation allows for targeted delivery to the lungs and systemic distribution through absorption in the respiratory tract. This route is suitable for substances targeting the respiratory system or requiring direct delivery to the lungs.

In some embodiments, a cell or therapeutic modality is administered via local administration. Local routes of administration in mice involve delivering drugs or therapeutic modalities directly to specific target tissues or regions of interest within the mouse body. These routes focus on localized delivery for localized effects, as opposed to systemic routes that aim for widespread distribution. Various local routes of administration are commonly used in mice for specific research objectives. Intradermal (ID) injection is a local route that delivers drugs or therapeutic modalities into the dermis, the layer of skin directly beneath the epidermis. This route is suitable for substances targeting the skin or requiring localized effects in the skin tissue. Subcutaneous (SC) injection, traditionally associated with systemic administration, can also be employed for local administration in mice. By targeting specific subcutaneous regions or anatomical sites, drugs or therapeutic modalities can be delivered directly to the desired local area. Intramuscular (IM) injection serves as both a systemic and local route of administration. In the context of local administration, the drug or therapeutic modality is injected directly into the muscle tissue at the specific site of interest. Intraperitoneal (IP) injection, primarily considered a systemic route, can also be utilized for local administration within the abdominal cavity. By delivering the drug or therapeutic modality into the peritoneal cavity, localized effects can be achieved in organs or tissues within the abdominal region. Intra-articular injection involves delivering drugs or therapeutic modalities directly into the joint space. Intranasal administration entails delivering drugs or therapeutic modalities through the nasal cavity. This local route allows for targeted effects in the nasal passages or the potential to target the central nervous system through the olfactory route. Topical administration involves the application of drugs or therapeutic modalities directly onto the skin or mucous membranes. This local route allows for localized effects on the skin or mucosal surfaces, such as the eyes, ears, or genitals.

In some embodiments, cells and/or agents are administered orthotopically. Orthotopic administration refers to the delivery of drugs or therapeutic modalities directly to the anatomically correct or appropriate location within an organism, mimicking the natural or original site of the disease or condition being studied. In the context of animal research, particularly in mice, orthotopic administration aims to reproduce the physiological and anatomical characteristics of a specific organ or tissue to study disease progression, treatment response, or other relevant biological processes. Orthotopic administration in mice involves various techniques to target specific organs or tissues. One commonly used approach is orthotopic tumor implantation, where tumor cells or tissues are injected or surgically placed directly into the corresponding anatomical site of interest. This method allows researchers to study tumor growth, metastasis, and treatment response in a manner that closely resembles the natural environment of the tumor. Another approach is organ-specific injection, where drugs or therapeutic modalities are delivered directly into a specific organ or tissue of interest. By injecting cells or other substances into organs like the liver, lungs, brain, or other organs, researchers can investigate organ-specific effects, disease models, or therapeutic interventions. Orthotopic transplantation is another technique used in mice, involving the surgical transfer or transplantation of tissues or cells to their anatomically correct location within the recipient mouse. This method is commonly used in transplantation studies to assess graft survival, integration, and functionality. Orthotopic infusion or instillation involves the direct introduction of substances into organs or cavities through a catheter or needle. For example, instilling drugs into the bladder or bronchi can mimic the physiological conditions of urinary or respiratory diseases, enabling researchers to study localized effects or treatment approaches.

In some embodiments, cells of the disclosure (e.g., human cells) are administered to a mammary fat pad of an immunodeficient mouse. The mammary fat pad of a mouse model refers to a specialized region of adipose (fat) tissue located within the mammary gland area of female mice. In female mice, the mammary glands are situated in pairs along the abdominal region. Each mammary gland is composed of multiple lobes and ductal structures embedded within the surrounding mammary fat pad.

In some embodiments, cells of the disclosure (e.g., human cells) are administered to a renal capsule of an immunodeficient mouse. The renal capsule of a mouse refers to the outer layer or covering that encapsulates the kidneys. It is a fibrous layer composed of connective tissue that surrounds and protects the kidneys, providing structural support. The renal capsule acts as a barrier, separating the kidneys from the surrounding tissues and organs. The renal capsule is often utilized for various procedures, including transplantation or implantation of cells, tissues, or therapeutic modalities into the kidney. This can involve making an incision in the renal capsule to access the kidney and perform the desired manipulation.

### Assays

In some embodiments, the methods further comprise assaying one or more effect(s) of the therapeutic modality on the human cells.

In some embodiments, the assaying comprises assaying for cell death (e.g., necrosis and/or apoptosis), inflammation, oxidative stress, alterations in cell morphology, alterations in cell function, accumulation of toxic substances, and changes in enzyme activity.

In some embodiments, the methods comprise assaying for cell death, which can lead to tissue damage and dysfunction. Cell death assays are used to measure and quantify different forms of cell death, such as apoptosis, necrosis, and autophagy. These assays help one understand the mechanisms and extent of cell death in various biological processes. Several commonly used cell death assays include the Annexin V/Propidium Iodide (PI) Assay, TUNEL Assay, Caspase Activity Assay, LDH Release Assay, MTT Assay, PI Exclusion Assay, and Caspase-Glo® Assays. The Annexin V/PI Assay distinguishes between early apoptotic and late-stage apoptotic or necrotic cells. Annexin V, labeled with a fluorescent marker, binds to phosphatidylserine, a marker for early apoptosis. Propidium iodide (PI) stains cells with compromised membranes, indicating late-stage apoptosis or necrosis. Flow cytometry is typically used to analyze the distribution of stained cells. The TUNEL (Terminal deoxynucleotidyl transferase dUTP nick end labeling) Assay detects DNA fragmentation, a characteristic of apoptosis. It involves labeling DNA strand breaks using a modified nucleotide that can be visualized using fluorescence microscopy or flow cytometry. This assay allows for the quantification of apoptotic cells within a population. Caspase Activity Assays measure the activity of specific caspases, enzymes involved in apoptosis. Using fluorescent or colorimetric substrates, these assays detect the cleavage of substrates by active caspases, generating a measurable signal. Caspase-3, -8, or -9 activity can be measured, indicating the activation of apoptotic pathways. The LDH (Lactate Dehydrogenase) Release Assay measures the release of LDH, an enzyme, into the culture medium upon cell membrane damage or disruption, which is characteristic of necrotic cell death. This assay quantifies the amount of LDH in the culture supernatant using a colorimetric or fluorometric assay, indicating compromised membrane integrity and cell death. The MTT (3-(4,5-Dimethylthiazol-2-yl)-2,5-diphenyltetrazolium Bromide) Assay measures cell viability based on the ability of living cells to reduce MTT, a yellow tetrazolium salt, to a purple formazan product. The formazan can be quantified spectrophotometrically, and a decrease in formazan production indicates reduced cell viability. The PI Exclusion Assay uses propidium iodide (PI), a DNA-intercalating fluorescent dye, to distinguish viable cells from non-viable ones. PI cannot penetrate intact cell membranes, so it only stains cells with compromised membrane integrity, such as necrotic cells. Flow cytometry or fluorescence microscopy can be used to analyze the stained cells. Caspase-Glo® Assays are luminescent assays that utilize a luminogenic caspase substrate. Upon caspase cleavage, the substrate emits a light signal. These assays can be specific to different caspases, such as caspase-3/7 or caspase-8, providing a sensitive and quantitative measurement of caspase activity, indicating apoptotic cell death.

In some embodiments, the methods comprise assaying for inflammation, which can lead to swelling, redness, and pain. Inflammation assays are widely used to study and measure the presence and extent of inflammation, a complex immune response that occurs in various tissues and organs. These assays help one understand the underlying mechanisms of inflammation, identify potential therapeutic targets, and evaluate the efficacy of anti-inflammatory treatments. Several commonly used inflammation assays include cytokine analysis, cell migration assays, leukocyte adhesion assays, nitric oxide assays, myeloperoxidase assays, histological staining, reactive oxygen species assays, and inflammatory gene expression analysis. Cytokine analysis is a key approach to quantify inflammation. This assay involves measuring the levels of specific cytokines, such as interleukins (IL), tumor necrosis factor-alpha (TNF-α), and interferons (IFN), in biological samples using techniques like ELISA, multiplex immunoassays, or protein arrays. By assessing cytokine profiles, one can gain insights into the inflammatory processes occurring in different tissues. Cell migration assays are utilized to study the migratory capacity of immune cells, such as neutrophils or monocytes, in response to inflammatory stimuli. Transwell assays or scratch assays provide valuable information about immune cell migration and infiltration into inflamed tissues. Leukocyte adhesion assays focus on measuring the adhesion of leukocytes (white blood cells) to endothelial cells, which is a critical step in the inflammatory response. By employing flow chamber assays or static adhesion assays, one can evaluate the adhesion properties of leukocytes under inflammatory conditions, contributing to our understanding of leukocyte-endothelial interactions. Nitric oxide (NO) assays are employed to measure the production of nitric oxide, a signaling molecule involved in inflammation. Griess reagent-based assays or fluorescent probes allow one to assess the levels of nitric oxide, which serves as an indicator of inflammatory activity. Myeloperoxidase (MPO) assays are used to quantify the presence of neutrophils or the extent of inflammation in tissues. MPO is an enzyme released by activated neutrophils and macrophages during inflammation, and measuring MPO activity provides insights into the level of neutrophil infiltration and inflammatory activity. Histological staining techniques, such as hematoxylin and eosin (H&E) staining, play a crucial role in visualizing and assessing inflammatory changes in tissue samples. By examining cellular and tissue alterations, including immune cell infiltration, tissue damage, and edema, one can identify and characterize inflammatory responses. Reactive oxygen species (ROS) assays detect the presence of reactive oxygen species generated during inflammation. Fluorescent probes, such as dichlorofluorescein diacetate (DCFDA), enable the measurement of ROS production in cells or tissues, indicating the presence and extent of inflammation. Inflammatory gene expression analysis involves quantifying the expression levels of specific inflammatory genes, including cytokines, chemokines, and adhesion molecules. Techniques such as quantitative real-time polymerase chain reaction (qPCR) or gene expression microarrays allow one to assess gene expression patterns, providing insights into the molecular aspects of the inflammatory response.

In some embodiments, the methods comprise assaying for oxidative stress, which can damage cellular components and cause tissue dysfunction. Oxidative stress assays are valuable tools used to measure and assess the levels of reactive oxygen species (ROS) and oxidative damage within cells and tissues. These assays provide insights into the oxidative stress status, which is implicated in various physiological and pathological conditions. Several commonly used oxidative stress assays include the DCFDA assay, NBT assay, total antioxidant capacity assay, lipid peroxidation assay, protein carbonyl assay, glutathione assay, DNA oxidation assay, and mitochondrial membrane potential assay. The DCFDA assay is a widely used fluorometric assay that measures intracellular ROS levels. DCFDA, a non-fluorescent probe, is oxidized by ROS to form the fluorescent compound dichlorofluorescein (DCF). The fluorescence intensity of DCF is proportional to the level of ROS within the cells and can be quantified using fluorescence microscopy or flow cytometry. The NBT assay detects superoxide anions, a type of ROS, by their ability to reduce NBT to formazan crystals. The intensity of the resulting blue formazan precipitate is proportional to the level of superoxide anions generated. This assay is commonly used in histochemical analysis to visualize and quantify superoxide production in tissues. Total antioxidant capacity assays measure the overall antioxidant capacity of biological samples, encompassing both enzymatic and non-enzymatic antioxidants. These assays evaluate the sample's ability to scavenge free radicals or prevent oxidative damage. Methods such as the Trolox equivalent antioxidant capacity (TEAC) assay and the ferric reducing antioxidant power (FRAP) assay are employed to determine the total antioxidant capacity. Lipid peroxidation assays assess the levels of lipid peroxidation products, such as malondialdehyde (MDA), as an indicator of oxidative damage to lipids. The thiobarbituric acid reactive substances (TBARS) assay or MDA assay is commonly used to measure lipid peroxidation, a common consequence of oxidative stress. Protein carbonyl assays detect the presence of carbonylated proteins, which result from protein oxidation due to oxidative stress. These assays derivatize the carbonyl groups with 2,4-dinitrophenylhydrazine (DNPH) and quantify the protein-bound DNPH, providing a measurement of protein oxidation using spectrophotometry. Glutathione assays evaluate the levels of reduced (GSH) and oxidized (GSSG) forms of glutathione, an important intracellular antioxidant. These assays, such as the enzymatic recycling method or Ellman's reagent-based assay, provide insights into the cellular antioxidant capacity and redox balance. DNA oxidation assays detect and quantify DNA damage resulting from oxidative stress. The comet assay (single-cell gel electrophoresis) and 8-hydroxy-2′-deoxyguanosine (8-OHdG) assay are commonly used to assess DNA damage, including oxidized bases and DNA strand breaks caused by oxidative stress. Mitochondrial membrane potential assays measure changes in mitochondrial function resulting from oxidative stress. Fluorescent dyes such as JC-1 or TMRE (tetramethylrhodamine ethyl ester) are employed to evaluate alterations in mitochondrial membrane potential using fluorescence microscopy or flow cytometry.

In some embodiments, the methods comprise assaying for alterations in cell morphology, for example, changes in the size, shape, and structure of cells, which can lead to tissue dysfunction. Assaying for alterations in cell morphology is an approach to study cellular changes associated with various biological processes or pathological conditions. By examining the structural characteristics and shape of cells, one can gain insights into cellular function, differentiation, disease progression, and response to treatments. Several commonly used methods enable the assessment of alterations in cell morphology. Light microscopy is a fundamental technique for visualizing and assessing cell morphology. Brightfield microscopy provides high-resolution images that allow one to examine overall cell shape, size, and features such as organelles and cytoplasmic structures. Phase contrast microscopy and differential interference contrast (DIC) microscopy enhance contrast and improve the visualization of cellular details, especially for transparent or unstained cells. Fluorescence microscopy utilizes fluorescent dyes or genetically encoded fluorescent proteins to label specific cellular components or structures. By targeting specific molecules, one can visualize and study alterations in cell morphology, such as changes in cytoskeletal organization, organelle distribution, or nuclear morphology. Techniques like immunofluorescence staining and live cell imaging provide valuable information about cellular dynamics and structural changes. Electron microscopy (EM) offers high-resolution imaging of cellular structures at the ultrastructural level. Transmission electron microscopy (TEM) provides detailed views of cellular organelles, membranes, and cytoplasmic components. Scanning electron microscopy (SEM) enables three-dimensional visualization of cell surfaces and can reveal alterations in cell shape, surface morphology, or the presence of cellular protrusions. Cytospin and cell smear techniques involve spreading cells onto glass slides, followed by fixation and staining. These methods allow one to examine cell morphology under a microscope and assess features such as cell size, shape, nuclear characteristics, and the presence of cellular inclusions or abnormalities. Staining techniques like Giemsa, Wright-Giemsa, or Papanicolaou stains can be employed to enhance cellular details and facilitate the identification of specific cell types. High-content imaging combines automated microscopy with image analysis software to quantitatively assess alterations in cell morphology and subcellular structures. This approach enables large-scale screening of cellular phenotypes, measuring parameters such as cell shape, size, texture, or fluorescent intensity. High-content imaging is particularly useful for studying cellular responses to treatments, genetic perturbations, or disease-related processes. Advanced image analysis software tools are available to quantify alterations in cell morphology from microscopy images. These tools allow one to measure parameters such as cell area, perimeter, circularity, aspect ratio, and intensity distribution. By comparing these morphological parameters between different experimental conditions or cell populations, one can identify and quantify changes in cell shape or structure.

In some embodiments, the methods comprise assaying for alterations in cell function, which can lead to tissue dysfunction and organ failure. Assaying for alterations in cell function is crucial for understanding cellular processes, evaluating the effects of treatments or genetic modifications, and investigating disease mechanisms. Various techniques and assays are available to assess changes in cellular function. These methods provide valuable insights into cellular behavior, signaling pathways, metabolism, and overall cellular health. Enzyme activity assays measure the activity levels of specific enzymes involved in various cellular processes. By employing specific substrates that undergo measurable changes upon enzymatic reactions, one can assess alterations in metabolic pathways, signal transduction, or other enzymatic processes. Calcium imaging techniques enable the monitoring of intracellular calcium levels, which play a critical role in cellular signaling and the regulation of various cellular functions. Fluorescence microscopy using calcium-sensitive dyes allows one to assess alterations in calcium dynamics, providing insights into processes such as neuronal signaling, muscle contraction, or cell communication. Electrophysiological techniques, such as patch-clamp recordings, measure the electrical activity of cells. These techniques assess alterations in membrane potential, ion channel activity, action potentials, synaptic transmission, or other electrical properties of cells. Electrophysiology is widely used in neuroscience and cardiac research to study cellular excitability and function. Metabolic assays measure various aspects of cellular metabolism, such as glucose uptake, ATP production, or oxygen consumption. By utilizing specific substrates or indicators, these assays allow one to quantify alterations in cellular energy metabolism or metabolic pathways. Cell proliferation and viability assays evaluate changes in cell growth, division, or survival. Techniques like MTT assays, cell counting, or live/dead staining provide quantitative or qualitative measurements of alterations in cell proliferation or viability in response to treatments, genetic modifications, or environmental conditions. Analyzing cell signaling pathways reveals changes in cellular responses or signaling cascades. Techniques such as Western blotting, immunofluorescence staining, or ELISA can be used to analyze protein expression, phosphorylation levels, or activation states of specific signaling molecules. These methods elucidate alterations in signaling pathways involved in processes like cell growth, differentiation, or immune responses. Functional imaging techniques, such as fMRI or PET, are used to study alterations in cell function in living organisms or tissues. These non-invasive imaging methods provide insights into functional changes in organs, tissues, or specific cell types and are commonly used in neuroscience, cardiovascular research, or oncology. Flow cytometry allows for the simultaneous analysis of multiple cellular parameters. By using fluorescently labeled antibodies or dyes, flow cytometry assesses alterations in cell surface markers, intracellular protein expression, cell cycle distribution, or apoptosis. It provides quantitative information on alterations in various cellular functions within complex cell populations.

In some embodiments, the methods comprise assaying for accumulation of toxic substances, which can lead to tissue damage and dysfunction. Assaying for the accumulation of toxic substances is essential for studying the impact of various chemicals, pollutants, or drugs on cells and organisms. These assays provide valuable insights into toxicological mechanisms, the potential adverse effects of substances, and the efficacy of detoxification or protective interventions. Several commonly used methods enable the assessment of toxic substance accumulation. Analytical techniques such as HPLC (High-Performance Liquid Chromatography) and GC-MS (Gas Chromatography-Mass Spectrometry) allow for the identification and quantification of toxic substances. HPLC separates and quantifies a wide range of compounds, providing information about their accumulation levels. GC-MS combines gas chromatography and mass spectrometry to detect and characterize toxic substances based on their mass-to-charge ratio, particularly for volatile or semi-volatile compounds. Fluorescence spectroscopy measures the emission of fluorescent light from a sample upon excitation with specific wavelengths. By using fluorescent probes or dyes, fluorescence spectroscopy can assess the accumulation of toxic substances by monitoring changes in fluorescence intensity or emission spectra. These probes selectively bind to or react with specific toxic compounds, offering a direct readout of their accumulation. Enzyme activity assays evaluate alterations in enzyme function caused by toxic substances. These assays employ specific substrates and indicators to measure enzyme activity, providing insights into the impact of toxic substances on cellular processes. Some toxic compounds can interfere with cellular enzymes, inhibiting their activity or leading to abnormal enzymatic reactions. Immunohistochemistry and immunofluorescence techniques use specific antibodies to detect and visualize the accumulation of toxic substances in tissues or cells. By targeting specific antigens or epitopes related to toxic compounds, these techniques allow for the spatial identification and localization of accumulated toxic substances. Cell-based assays utilize specific fluorescent dyes or probes to assess the accumulation of toxic substances in cultured cells. These assays employ fluorescence microscopy or flow cytometry to quantify the accumulation of toxic compounds, offering insights into their cellular uptake, distribution, and metabolism. Tissue analysis can be employed to study the accumulation of toxic substances in vivo. Tissue analysis involves the extraction and quantification of toxic compounds from organs or biological fluids, enabling one to evaluate their accumulation levels and distribution patterns in different tissues or body compartments. Indirect assays target specific physiological or biochemical changes caused by toxic substances. Assays measuring oxidative stress markers, DNA damage, or metabolic alterations can indirectly infer the presence and accumulation of toxic compounds. These changes serve as indicators of the effects of toxic substances on cells or organisms.

In some embodiments, the methods comprise assaying for changes in enzyme activity, which can lead to tissue dysfunction and organ failure. Assaying for changes in enzyme activity can be used for studying enzymatic processes, assessing the impact of various factors on enzyme function, and identifying potential disease-related alterations. Several commonly used methods allow one to quantitatively measure the catalytic activity of enzymes and detect changes in their function. Spectrophotometric assays utilize the measurement of absorbance or color change to quantify enzyme activity. These assays often involve enzymatic reactions that produce or consume specific substrates, resulting in changes in light absorption. By monitoring the absorbance or color intensity, one can determine the rate of enzymatic activity. Examples include the use of substrates such as NADH or NADPH, which exhibit changes in absorbance upon enzymatic reactions. Fluorometric assays rely on the detection of fluorescence emitted by a substrate or product of an enzymatic reaction. Fluorescent molecules can be designed to interact specifically with certain enzymes, generating fluorescence signals upon enzymatic activity. By measuring the fluorescence intensity, one can quantify enzyme activity. Fluorometric assays are highly sensitive and often used in high-throughput screening. Radiometric assays involve the use of radioactive isotopes to track enzymatic reactions. Radioactive substrates or cofactors are used in the enzymatic reaction, and the radioactivity of the reaction products is measured using techniques such as liquid scintillation counting. These assays provide high sensitivity but require special precautions due to the use of radioactive materials. Enzyme-Linked Immunosorbent Assay (ELISA) utilizes the specificity of antibodies to detect and quantify enzyme activity. In these assays, enzymes are conjugated to antibodies or antigens, and their activity is measured through the detection of an enzymatic reaction product. ELISA is widely used for the quantification of various enzymes or enzyme activities in biological samples. Gel electrophoresis techniques, such as zymography or native gel electrophoresis, are used to assess changes in enzyme activity based on their mobility in a gel matrix. Enzymes are separated based on their size, charge, or activity, and subsequent staining or activity-based detection methods reveal alterations in enzyme activity. Kinetic assays measure the rate of enzyme-catalyzed reactions under varying substrate concentrations or reaction conditions. These assays determine key kinetic parameters such as the Michaelis-Menten constant (Km) and maximum reaction velocity (Vmax), providing insights into enzyme-substrate interactions and the impact of factors on enzyme activity. Common kinetic assays include the Lineweaver-Burk plot and steady-state kinetic analysis. Mass spectrometry can be utilized to quantify enzyme activity by measuring the consumption or production of metabolites involved in enzymatic reactions. Isotope-labeled substrates or reactants can be introduced, and the change in isotopic ratio is detected using mass spectrometry. This approach allows for precise measurements of enzyme activity and can be applied to complex enzymatic pathways. Activity-based probes are small molecules that selectively react with active enzyme sites. These probes covalently modify active enzymes, allowing for their subsequent detection or isolation. Activity-based probes provide a powerful method for profiling enzyme activity in complex biological systems.

In some embodiments, the methods comprise assaying for increased survival. In some embodiments, the methods comprise assaying for improved symptoms. In some embodiments, the methods comprise assaying for improved overall health.

### Additional Aspects

Additional aspects of the present disclosure are provided in the following numbered paragraphs.

1. An immunodeficient mouse comprising:
an endogenous H2-K allele comprising a null mutation, an endogenous H2-D allele comprising a null mutation, an endogenous H2-A allele comprising a null mutation, and an exogenous nucleic acid selected from a nucleic acid encoding human interleukin 7 (huIL7) and a nucleic acid encoding human interleukin 15 (huIL15).
   2. The immunodeficient mouse of paragraph 1 comprising the nucleic acid encoding huIL7 and the nucleic acid encoding huIL15.
   3. The immunodeficient mouse of paragraph 1, wherein the endogenous H2-K1 allele comprising a null mutation is a H2-K1^{tm1Bpe} allele.
   4. The immunodeficient mouse of paragraph 1, wherein the endogenous H2-D allele comprising a null mutation is a H2-D1^{tm1Bpe} allele.
   5. The immunodeficient mouse of paragraph 1, wherein the endogenous H2-A allele comprising a null mutation is an H2-Ab1^{em1Mvw} allele.
   6. The immunodeficient mouse of any one of the preceding paragraphs, further comprising an endogenous Interleukin 2 Receptor Gamma (Il2rg) allele comprising a null mutation.
   7. The immunodeficient mouse of paragraph 6, wherein the endogenous Il2rg allele comprising a null mutation is an Il2rg^{tm1Wjl} allele.
   8. The immunodeficient mouse of paragraph 6, wherein the endogenous Il2rg allele comprising a null mutation is an Il2rg^{tm1Sug} allele.
   9. The immunodeficient mouse of any one of the preceding paragraphs, further comprising an endogenous Protein Kinase, DNA-Activated, Catalytic Subunit (Prkdc) allele comprising a null mutation.
   10. The immunodeficient mouse of paragraph 9, wherein mutation is a severe combined immunodeficiency (scid) mutation.
   11. The immunodeficient mouse of paragraph 10, wherein the endogenous Prkdc allele comprising a null mutation is a Prkdc^{scid} allele.
   12. The immunodeficient mouse of any one of the preceding paragraphs further comprising an endogenous Recombination Activating Gene 1 (Rag1) allele comprising a null mutation.
   13. The immunodeficient mouse of paragraph 12, wherein the endogenous Rag1 allele comprising a null mutation is a Rag^{tm1Mom} allele.
   14. The immunodeficient mouse of any one of the preceding paragraphs, further comprising an endogenous Recombination Activating Gene 2 (Rag2) allele comprising a null mutation.
   15. The immunodeficient mouse of paragraph 14, wherein the endogenous Rag2 allele comprising a null mutation is a Rag2^{tm1Fwa} allele.
   16. The immunodeficient mouse of any one of the preceding paragraphs, wherein the immunodeficient mouse has a non-obese diabetic (NOD) background.
   17. The immunodeficient mouse of paragraph 16, wherein the immunodeficient mouse has a NOD scid gamma background.
   18. The immunodeficient mouse of paragraph 16 or 17, wherein the immunodeficient mouse has an NSG-(K^{b} D^{b})^{null} (IA^{null}) genetic background.
   19. The immunodeficient mouse of any one of paragraphs the preceding paragraphs, wherein the mouse further comprises an exogenous nucleic acid selected from a nucleic acid encoding human interleukin 3 (huIL3), a nucleic acid encoding human granulocyte/macrophage-stimulating factor (huGM-CSF), and a nucleic acid encoding human Steel factor (huSCF).
   20. The immunodeficient mouse of paragraph 17, wherein the mouse further comprises the nucleic acid encoding huIL3, the nucleic acid encoding huGM-CSF, and the nucleic acid encoding huSCF.
   21. The immunodeficient mouse of any one of the preceding paragraphs, wherein the immunodeficient mouse further comprises an endogenous Kit allele comprising a null mutation.
   22. The immunodeficient mouse of paragraph 21, wherein the endogenous Kit allele comprising a null mutation is Kit^{W-41J}.
   23. The immunodeficient mouse of any one of the preceding paragraphs, wherein the mouse further comprises (or has been engrafted with) human cells.
   24. The immunodeficient mouse of paragraph 23, wherein the mouse further comprises (or has been engrafted with) unfractionated human umbilical cord blood comprising the human cells.
   25. The immunodeficient mouse of paragraph 23 or 24, wherein the human cells comprise human hematopoietic stem cells.
   26. The immunodeficient mouse of any one of paragraphs 23-25, wherein the human cells comprise human peripheral blood mononuclear cells.
   27. The immunodeficient mouse of the preceding paragraphs, wherein the human cells are not enriched for CD34⁺ human hematopoietic stem cells.
   28. The immunodeficient mouse of the preceding paragraphs, wherein CD3⁺ human T cells have not been depleted from the human cells.
   29. The immunodeficient mouse of any one of the preceding paragraphs, wherein the mouse has not been subjected to myeloablation, optionally irradiation or chemical myeloablation.
   30. A method for producing a humanized mouse, the method comprising administering human cells to the immunodeficient mouse of any one of paragraphs 1-22.
   31. The method of paragraph 30, wherein the method comprises administering unfractionated human umbilical cord blood comprising the human cells.
   32. The method of paragraph 30 or 31, wherein the human cells comprise human hematopoietic stem cells.
   33. The method of any one of paragraphs 30-32, wherein the human cells comprise human hematopoietic peripheral blood mononuclear cells.
   34. A method of producing the immunodeficient mouse of any one of the preceding paragraphs, the method comprising breeding:
(i) a mouse comprising an endogenous Kit allele comprising a null mutation, an exogenous nucleic acid encoding human interleukin 3 (huIL3), an exogenous nucleic acid encoding human granulocyte-macrophage colony-stimulating factor (huGM-CSF), and an exogenous nucleic acid encoding human Steel factor (huSCF); and
(ii) a mouse comprising an endogenous H2-K allele comprising a null mutation, an endogenous H2-D allele comprising a null mutation, an endogenous H2-A allele comprising a null mutation, an exogenous nucleic acid encoding human interleukin 7 (huIL7), and an exogenous nucleic acid encoding human interleukin 15 (huIL15).
   35. The method of paragraph 34, wherein the immunodeficient mouse is homozygous for the endogenous Kit allele comprising a null mutation.
   36. The method of paragraph 35 or 36, wherein the immunodeficient mouse is homozygous for the endogenous H2-K allele comprising a null mutation, homozygous for the endogenous H2-D allele comprising a null mutation, and/or homozygous for the endogenous H2-A allele comprising a null mutation.
   37. A method comprising: administering human cells to the immunodeficient mouse of any one of the preceding paragraphs.
   38. The method of paragraph 37, wherein the method further comprises administering unfractionated human umbilical cord blood comprising the human cells.
   39. The method of paragraph 37 or 38, wherein the human cells comprise human hematopoietic stem cells.
   40. The method of any one of paragraphs 37-39, wherein the human cells comprise human peripheral blood mononuclear cells.
   41. The method of any one of paragraphs 37-40, wherein the human cells are not enriched for CD34⁺ human hematopoietic stem cells.
   42. The method of any one of paragraphs 37-41, wherein CD3⁺ human T cells have not been depleted from the human cells.
   43. The method of any one of paragraphs 37-42, wherein the method excludes subjecting the immunodeficient mouse to myeloablation, optionally irradiation or chemical myeloablation.
   44. A mouse model having the genotype/strain described in Table 1.
   45. A progeny mouse of any one of the matings described in Examples 1-5.
   46. An immunodeficient non-obese diabetic (NOD) mouse comprising:
an endogenous Il2rg allele comprising a null mutation;
an endogenous H2-K allele comprising a null mutation;
an endogenous H2-D allele comprising a null mutation;
an endogenous H2-A allele comprising a null mutation;
an endogenous Kit allele comprising a null mutation;
an exogenous nucleic acid encoding human interleukin 7 (huIL7);
an exogenous nucleic acid encoding human interleukin 15 (huIL15);
an exogenous nucleic acid encoding human interleukin 3 (huIL3);
an exogenous nucleic acid encoding human granulocyte-macrophage colony-stimulating factor (huGM-CSF); and
an exogenous nucleic acid encoding human Steel factor (huSCF).
   47. The immunodeficient mouse of paragraph 46, wherein the endogenous H2-K1 allele comprising a null mutation is a H2-K1^{tm1Bpe} allele.
   48. The immunodeficient mouse of paragraph 46 or 47, wherein the endogenous H2-D allele comprising a null mutation is a H2-D1^{tm1Bpe} allele.
   49. The immunodeficient mouse of any one of the preceding paragraphs, wherein the endogenous H2-A allele comprising a null mutation is an H2-Ab1^{em1Mvw} allele.
   50. The immunodeficient mouse of any one of the preceding paragraphs, wherein the endogenous Il2rg allele comprising a null mutation is an Il2rg^{tm1Wjl} allele.
   51. The immunodeficient mouse of any one of paragraphs 46-49, wherein the endogenous Il2rg allele comprising a null mutation is an Il2rg^{tm1Sug} allele.
   52. The immunodeficient mouse of any one of the preceding paragraphs, further comprising an endogenous Prkdc allele comprising a null mutation.
   53. The immunodeficient mouse of paragraph 52, wherein the null mutation in the endogenous Prkdc allele is a severe combined immunodeficiency (scid) mutation.
   54. The immunodeficient mouse of paragraph 53, wherein the endogenous Prkdc allele comprising a null mutation is a Prkdc^{scid} allele.
   55. The immunodeficient mouse of any one of the preceding paragraphs further comprising an endogenous Recombination Activating Gene 1 (Rag1) allele comprising a null mutation.
   56. The immunodeficient mouse of paragraph 55, wherein the endogenous Rag1 allele comprising a null mutation is a Rag^{tm1Mom} allele.
   57. The immunodeficient mouse of any one of the preceding paragraphs, further comprising an endogenous Recombination Activating Gene 2 (Rag2) allele comprising a null mutation.
   58. The immunodeficient mouse of paragraph 12, wherein the endogenous Rag2 allele comprising a null mutation is a Rag2^{tm1Fwa} allele.
   59. The immunodeficient mouse of any one of the preceding paragraphs, wherein the immunodeficient mouse has a NOD scid gamma background.
   60. The immunodeficient mouse of paragraph 59, wherein the immunodeficient mouse has an NSG-(K^{b} D^{b})^{null} (IA^{null}) genetic background.
   61. The immunodeficient mouse of any one of the preceding paragraphs, wherein the endogenous Kit allele comprising a null mutation is Kit^{W-41J}.
   62. The immunodeficient mouse of any one of the preceding paragraphs, wherein the mouse further comprises (or has been engrafted with) human cells.
   63. The immunodeficient mouse of paragraph 62, wherein the mouse further comprises (or has been engrafted with) unfractionated human umbilical cord blood comprising the human cells.
   64. The immunodeficient mouse of paragraph 62 or 63, wherein the human cells comprise human hematopoietic stem cells.
   65. The immunodeficient mouse of any one of paragraphs 62-64, wherein the human cells comprise human peripheral blood mononuclear cells.
   66. The immunodeficient mouse of any one of the preceding paragraphs, wherein the human cells are not enriched for CD34⁺ human hematopoietic stem cells.
   67. The immunodeficient mouse of any one of the preceding paragraphs, wherein CD3⁺ human T cells have not been depleted from the human cells.
   68. The immunodeficient mouse of any one of the preceding paragraphs, wherein the mouse has not been subjected to myeloablation, optionally irradiation or chemical myeloablation.
   69. A method for producing a humanized mouse, the method comprising administering human cells to the immunodeficient mouse of any one of paragraphs 46-61.
   70. The method of paragraph 69, wherein the method comprises administering unfractionated human umbilical cord blood comprising the human cells.
   71. The method of paragraph 69 or 70, wherein the human cells comprise human hematopoietic stem cells.
   73. The method of any one of paragraphs 69-71, wherein the human cells comprise human hematopoietic peripheral blood mononuclear cells.
   73. A method of producing the immunodeficient mouse of any one of the preceding paragraphs, the method comprising breeding:
(i) a mouse comprising an endogenous Kit allele comprising a null mutation, an exogenous nucleic acid encoding human interleukin 3 (huIL3), an exogenous nucleic acid encoding human granulocyte-macrophage colony-stimulating factor (huGM-CSF), and an exogenous nucleic acid encoding human Steel factor (huSCF); and
(ii) a mouse comprising an endogenous H2-K allele comprising a null mutation, an endogenous H2-D allele comprising a null mutation, an endogenous H2-A allele comprising a null mutation, an exogenous nucleic acid encoding human interleukin 7 (huIL7), and an exogenous nucleic acid encoding human interleukin 15 (huIL15).
   74. The method of paragraph 73, wherein the immunodeficient mouse is homozygous for the endogenous Kit allele comprising a null mutation.
   75. The method of paragraph 73 or 74, wherein the immunodeficient mouse is homozygous for the endogenous H2-K allele comprising a null mutation, homozygous for the endogenous H2-D allele comprising a null mutation, and/or homozygous for the endogenous H2-A allele comprising a null mutation.
   76. A method comprising:
administering human cells to the immunodeficient mouse of any one of the preceding paragraphs.
   77. The method of paragraph 76, wherein the method further comprises administering unfractionated human umbilical cord blood comprising the human cells.
   78. The method of paragraph 76 or 77, wherein the human cells comprise human hematopoietic stem cells.
   79. The method of any one of paragraphs 76-78, wherein the human cells comprise human peripheral blood mononuclear cells.
   80. The method of any one of paragraphs 76-79, wherein the human cells are not enriched for CD34⁺ human hematopoietic stem cells.
   81. The method of any one of paragraphs 76-80, wherein CD3⁺ human T cells have not been depleted from the human cells.
   82. The method of any one of paragraphs 76-81, wherein the method excludes subjecting the immunodeficient mouse to myeloablation, optionally irradiation or chemical myeloablation.

### EXAMPLES

TABLE 1
NSG ® Mouse Strains

| # | Strain Name | Purpose |
|---|---|---|
| 1 | NSG ® | Foundation strain |
| | NOD.Cg-Prkdc^{scid} Il2rg^{tm1Wjl}/SzJ | |
| 2 | NSG-MHC class I/II KO | Engrafting with human PBMC or |
| | “NSG-MHC DKO” | unfractionated umbilical cord blood (UCB) |
| | NOD.Cg-Prkdc^{scid} H2-K1^{tm1Bpe} H2-Ab1^{em1Mvw} H2- | without causing acute GVHD |
| | D1^{tm1Bpe} Il2rg^{tm1Wjl}/SzJ | |
| 3 | NSG-Tg(Hu-IL7) | Enhance development, survival and function of |
| | | human T cells |
| 4 | NSG-Tg(Hu-IL15) | Enhance development, survival and function of |
| | NOD.Cg-Prkdc^{scid} Il2rg^{tm1Wjl} Tg(IL15)1Sz/SzJ | human NK cells |
| 5 | NSG-Tg(Hu-IL7)(Hu-IL15) | Enhance development, survival and function of |
| | | human T and NK cells |
| 6 | NSG-W41 Tg(SGM3) | Enhance engraftment and development of |
| | | myeloid cells without the need for |
| | | preconditioning |
| 7 | NSG-MHC DKO Tg(Hu-IL15) | Enhance human NK cell expansion and |
| | | survival following human PBMC or |
| | | unfractionated cord blood engraftment |
| 8 | NSG-Tg(Hu-IL15)(SGM3) | Enhance both human NK cell and myeloid cell |
| | | development following human HSC |
| | | engraftment |
| 9 | NSG-MHC DKO Tg(Hu-IL7)(Hu-IL15) | Enhance survival and function of human NK |
| | | cells and T cells in the absence of GVHD |
| 10 | NSG-MHC DKO Tg(Hu-IL15)(SGM3) | Enhance development survival and function of |
| | | human NK cells and myeloid cells in the |
| | | absence of GVHD |
| 11 | NSG-MHC DKO W41 Tg(Hu-IL7)(Hu-IL15)(SGM3) | Enhance development, survival and function of |
| | | human NK and T cells, and myeloid cells in |
| | | the absence of GVHD and permit engraftment |
| | | of HSC in the absence of preconditioning |
| 12 | NSG-MHC DKO Tg(Hu-IL7)(Hu-IL15)(SGM3) | Enhance development, survival and function of |
| | | human NK cells, T cells, and myeloid cells in |
| | | the absence of GVHD |
| 13 | NSG-MHC DKO Tg(Hu-IL7)(SGM3) | Enhance development, survival and function of |
| | | human T cells, and myeloid cells in the |
| | | absence of GVHD |
| 14 | NSG-SGM3 | Enhanced engraftment of myeloid lineages and |
| | NOD.Cg-Prkdc^{scid} Il2rg^{tm1Wjl} Tg(CMV- | regulatory T cell populations |
| | IL3, CSF2, KITLG)1Eav/MloySzJ | |

Tg: transgene/transgenic
SGM3: Transgene encoding human interleukin 3 (huIL3); Transgene encoding human granulocyte-macrophage colony-stimulating factor (huGM-CSF); Transgene encoding human stem cell factor (huSCF)
W41: Kit^{W-41J} mutant allele

### Description of NSG® Mouse Strains in Table 1:

Strain 1 (NSG®): severely immunodeficient platform strain for all the models in this study—see RRID:IMSR_JAX:005557.

Strain 2 (NSG-MHC DKO): lacks expression of major histocompatibility class I and class II genes and supports engraftment with human peripheral blood mononuclear cells (PBMC) or unfractionated human cord blood without the development of acute xenogeneic graft versus host disease (GVHD)—see RRID:IMSR_JAX:025216.

Strain 3 (NSG-Tg(Hu-IL7)): provides expression of the human IL7 transgene which helps T cell, NK cell and other lymphoid cell development and survival. This strain was made using Clone RP11-19N15 ordered from CHORI BACPAC. The only gene contained in the BAC was human IL7. The BAC is 164110bps long. It runs from 79630616-79794725 on chromosome 8 q21.12. Accession end numbers are B85558 (plus strand) and AQ14183 (minus strand). It was grown in LB+ chloramphenicol, and DNA was purified using the Qiagen Large Construct Kit. The product was sequenced before being injected into NOD+/scid embryos.

Three out of 84 potential founders typed positive for the transgene. They were all females. All were mated with male NSG® mice and had large litters. One of the lines was fixed to homozygosity.

Strain 4 (NSG-Tg(Hu-IL15)): provides expression of human IL15 which supports human natural killer (NK) cell development and survival and also helps human T cell survival. This strain was generated using a 200 Kbp BACPAC obtained from ChoriBACPAC. Pronuclear injection yielded a male transgenic founder that transmitted the transgene to his offspring. NSG-Tg(Hu-IL15) mice produce physiological levels of human IL15—see RRID:IMSR_JAX:030890.

Strain 5 (NSG-Tg(Hu-IL7)(Hu-IL15)): This strain doubly expresses human IL7 and IL15.

Strain 6 (NSG-Tg(SGM3) W41): CRISPR Cas9 was used to create a G to A point mutation to create the W41 mutation in the Kit gene in NSG mice. This strain supports human HSC engraftment with the need to irradiate the recipients.

Strain 7 (NSG-MHC DKO Tg(Hu-IL15)): This strain expresses human IL15 in the absence of mouse MHC class I and II.

Strain 8 (NSG-Tg(Hu-IL15) Tg(Hu-SGM3)): This strain expresses human IL15 as well as human stem cell factor, human IL3 and human GM-CSF.

Strain 9 (NSG-MHC DKO Tg(Hu-IL7)(Hu-IL15)): This strain expresses human IL7 and human IL15 in the absence of mouse MHC class I and II.

Strain 10 (NSG-MHC DKO Tg(Hu-IL15) (SGM3)): This strain expresses human IL15, as well as human stem cell factor, human IL3 and human GM-CSF in the absence of mouse MHC class I and II.

Strain 11 (NSG-MHC DKO Tg(Hu-IL7) (Hu-IL15) (SGM3) W41): This strain expresses human IL7 and human IL15, as well as human stem cell factor, human IL3 and human GM-CSF in the absence of mouse MHC class I and II and also expresses the W41 mutation.

Strain 12 (NSG-MHC DKO Tg(Hu-IL7) (Hu-IL15) (SGM3)): This strain expresses human IL7 and human IL15, as well as human stem cell factor, human IL3 and human GM-CSF in the absence of mouse MHC class I and II.

Strain 13 (NSG-MHC DKO Tg(Hu-IL7) (SGM3)): This strain expresses human IL7 and human stem cell factor, human IL3 and human GM-CSF in the absence of mouse MHC class I and II.

Strain 14 (NSG-SGM3): This triple transgenic strain expresses human IL3, GM-CSF (CSF2) and SCF (KITLG), cytokines that support the stable engraftment of myeloid lineages and regulatory T cell populations, allowing superior engraftment of diverse hematopoietic lineages—see RRID:IMSR_JAX:013062.

Strains 9-13 are being generated by genetic crosses described below using NSG® mouse model strains 1-8. Mice produced in each mating for the crosses described below should be genotyped by PCR, RT-PCR or melting curves as appropriate for each genotype.

### Example 1: Generation of NSG-MHC DKO Tg(Hu-IL7)(Hu-IL15) Mice (Strain 9)

### Mating 1

$$NSG ‐ Tg ( Hu ‐ IL 7 / Hu ‐ IL 7 ) ( Hu ‐ IL 15 / Hu ‐ IL 15 ) ( Strain 5 ) \times NSG ‐ ( MHC DKO / MHC DKO ) Tg ( Hu ‐ IL 15 / Hu ‐ IL 15 ) ( Strain 7 )$$

100% of the progeny mice should be heterozygous for the MHC class I/II double knockout (MHC DKO), hemizygous for the human IL7 (Hu-IL7) transgene, and homozygous for the human IL-15 (Hu-IL15) transgene:

$$NSG ‐ ( + / MHC DKO ) Tg ( + / Hu ‐ IL 7 ) ( Hu ‐ IL 15 / Hu ‐ IL 15 ) ( Mating 1 Progeny )$$

### Mating 2

$$NSG - ( + / MHC DKO ) Tg ( + / Hu - IL 7 ) ( Hu - IL 15 / Hu - IL 15 ) ( Mating 1 Progeny ) \times NSG - ( MHC DKO / MHC DKO ) Tg ( Hu - IL 15 / Hu - IL 15 ) ( Strain 7 )$$

25% of the progeny mice should be homozygous for MHC DKO, hemizygous for the Hu-IL-7 transgene, and homozygous for the Hu-IL15 transgene:

$$NSG - ( MHC DKO / MHC DKO ) Tg ( + / Hu - IL 7 ) ( Hu - IL 15 / Hu - IL 15 ) ( Mating 2 Progeny )$$

### Mating 3

$$NSG - ( MHC DKO / MHC DKO ) Tg ( + / Hu - IL 7 ) ( Hu - IL 15 / Hu - IL 15 ) ( Mating 2 Progeny ) \times NSG - ( MHC DKO / MHC DKO ) Tg ( + / Hu - IL 7 ) ( Hu - IL 15 / Hu - IL 15 ) ( Mating 2 Progeny )$$

12.5% of the progeny mice should be homozygous for the MHC DKO, hemizygous for the Hu-IL7 transgene, and homozygous for the Hu-IL15 transgene:

$$NSG - ( MHC DKO / MHC DKO ) Tg ( + / Hu - IL 7 ) ( Hu - IL 15 / Hu - IL 15 ) ( Mating 3 Progeny A )$$

12.5% of the progeny mice should be homozygous for the MHC DKO, homozygous for the Hu-IL7 transgene, and homozygous for the Hu-IL15 transgene:

$$NSG - ( MHC DKO / MHC DKO ) Tg ( Hu - IL 7 / Hu - IL 7 ) ( Hu - IL 15 / Hu - IL 15 ) ( Mating 3 Progeny B )$$

### Mating 4

$$NSG - ( MHC DKO / MHC DKO ) Tg ( + / Hu - IL 7 ) ( Hu - IL 15 / Hu - IL 15 ) ( Mating 3 Progeny A ) \times NSG - ( MHC DKO / MHC DKO ) Tg ( Hu - IL 7 / Hu - IL 7 ) ( Hu - IL 15 / Hu - IL 15 ) ( Mating 3 Progeny B )$$

50% of the progeny mice should be homozygous for the MHC DKO, homozygous for the Hu-IL7 transgene, and homozygous for the Hu-IL15 transgene:

$$NSG - ( MHC DKO / MHC DKO ) Tg ( Hu - IL 7 / Hu - IL 7 ) ( Hu - IL 15 / Hu - IL 15 ) ( Mating 4 Progeny )$$

### Mating 5

$$NSG - ( MHC DKO / MHC DKO ) Tg ( Hu - IL 7 / Hu - IL 7 ) ( Hu - IL 15 / Hu - IL 15 ) ( Mating 4 Progeny ) \times NSG \text{} - ( MHC DKO / MHC DKO ) Tg ( Hu - IL 7 / Hu - IL 7 ) ( Hu - IL 15 / Hu - IL 15 ) ( Mating 4 Progeny )$$

The NSG-(MHC DKO/MHC DKO) Tg(Hu-IL7/Hu-IL7)(Hu-IL15/Hu-IL15) progeny strain will be maintained by sib mating.

### Example 2: Generation of NSG-MHC DKO Tg(Hu-IL15)(SGM3) (Strain 10)

### Mating 1

$$NSG - Tg ( Hu - IL 15 / Hu - IL 15 ) ( SGM 3 / SGM 3 ) ( Strain 8 ) \times NSG \text{} - ( MHC DKO / MHC DKO ) Tg ( Hu - IL 15 / Hu - IL 15 ) ( Strain 7 )$$

100% of the progeny mice should be heterozygous for the MHC DKO, homozygous for the Hu-IL15 transgene, and hemizygous for the human SGM3 (SMG3) transgenes:

$$NSG - ( + / MHC DKO ) Tg ( Hu - IL 15 / Hu - IL 15 ) ( + / SGM 3 ) ( Mating 1 Progeny )$$

### Mating 2

$$NSG - ( + / MHC DKO ) Tg ( Hu - IL 15 / Hu - IL 15 ) ( + / SGM 3 ) ( Mating 1 Progeny ) \times NSG - ( MHC DKO / MHC DKO ) Tg ( Hu - IL 15 / Hu - IL 15 ) ( Strain 7 )$$

25% of the progeny mice should be homozygous for the MHC DKO, homozygous for the Hu-IL15 transgene, and hemizygous for the human SGM3 (SMG3) transgenes:

$$NSG - ( MHC DKO / MHC DKO ) Tg ( Hu - IL 15 / Hu - IL 15 ) ( + / SGM 3 ) ( Mating 2 Progeny )$$

### Mating 3

$$NSG - ( MHC DKO / MHC DKO ) Tg ( Hu - IL 15 / Hu - IL 15 ) ( + / SGM 3 ) ( Mating 2 Progeny ) \times \\ NSG - ( MHC DKO / MHC DKO ) Tg ( Hu - IL 15 / Hu - IL 15 ) ( + / SGM 3 ) ( Mating 2 Progeny )$$

25% of the progeny mice should be homozygous for the MHC DKO, homozygous for the Hu-IL15 transgene, and homozygous for the human SGM3 (SMG3) transgenes:

$$NSG - ( MHC DKO / MHC DKO ) Tg ( Hu - IL 15 / Hu - IL 15 ) ( SGM 3 / SGM 3 ) ( Mating 3 Progeny A )$$

50% of the progeny mice should be heterozygous for the MHC DKO, homozygous for the Hu-IL15 transgene, and hemizygous for the human SGM3 (SMG3) transgenes:

$$NSG - ( MHC DKO / MHC DKO ) Tg ( Hu - IL 15 / Hu - IL 15 ) ( + / SGM 3 ) ( Mating 3 Progeny B )$$

### Mating 4

$$NSG - ( MHC DKO / MHC DKO ) Tg ( Hu - IL 15 / Hu - IL 15 ) ( SGM 3 / SGM 3 ) ( Mating 3 Progency A ) \times NSG - ( MHC DKO / MHC DKO ) Tg ( Hu - IL 15 / Hu - IL 15 ) ( + / SGM 3 ) ( Mating 3 Progency B )$$

50% of the progeny mice should be homozygous for the MHC DKO, homozygous for the Hu-IL15 transgene, and homozygous for the human SGM3 (SMG3) transgenes:

$$NSG - ( MHC DKO / MHC DKO ) Tg ( Hu - IL 15 / Hu - IL 15 ) ( SGM 3 / SGM 3 ) ( Mating 4 Progency )$$

### Mating 5

$$NSG - ( MHC DKO / MHC DKO ) Tg ( Hu - IL 15 / Hu - IL 15 ) ( SGM 3 / SGM 3 ) ( Mating 4 Progency ) \times NSG - ( MHC DKO / MHC DKO ) Tg ( Hu - IL 15 / Hu - IL 15 ) ( SGM 3 / SGM 3 ) ( Mating 4 Progency )$$

The NSG-MHC DKO/MHC DKO) Tg(Hu-IL15/Hu-IL15)(SGM3/SGM3) progeny strain will be maintained by sib mating.

### Example 3: Generation of NSG-MHC DKO W41 Tg(Hu-IL15)(Hu-IL7)(SGM3) (Strain 11)

### Mating 1

$$NSG - ( + / MHC DKO ) Tg ( + / Hu - IL 7 ) ( Hu - IL 15 / Hu - IL 15 ) ( Strain 9 Female Mating 1 Progeny ) \times NSG - ( W 41 / W 41 ) Tg ( SGM 3 / SGM 3 ) ( Strain 6 Males )$$

25% of the progeny mice should be heterozygous for the MHC DKO, heterozygous for the Kit^{W-41J}mutant (W41) allele, hemizygous for the Hu-IL7 transgene, hemizygous for the Hu-IL15 transgene, and hemizygous for the SGM3 transgenes:

$$NSG - ( + / MHC DKO ) ( + / W 41 ) Tg ( + / Hu - IL 7 ) ( + / Hu - IL 15 ) ( + / SGM 3 ) ( Mating 1 Progeny )$$

### Mating 2

$$NSG - ( + / MHC DKO ) ( + / W 41 ) Tg ( + / Hu - IL 7 ) ( + / Hu - IL 15 ) ( + / SGM 3 ) ( Mating 1 Progeny ) \times NSG - MHC DKO / MHC DKO ) Tg ( Hu - IL 15 / Hu - IL 15 ) ( SGM 3 / SGM 3 ) ( Strain 10 )$$

3.125% of the progeny mice should be homozygous for the MHC DKO, heterozygous for the Kit^{W-41J}mutant (W41) allele, hemizygous for the Hu-IL7 transgene, homozygous for the Hu-IL15 transgene, and homozygous for the SGM3 transgenes:

$$NSG - ( MHC DKO / MHC DKO ) ( + / W 41 ) Tg ( + / Hu - IL 7 ) ( Hu - IL 15 ) ( SGM 3 / SGM 3 ) ( Mating 2 Progeny )$$

### Mating 3

$$NSG - ( MHC DKO / MHC DKO ( + / W 41 ) Tg ( \text{} + / Hu - IL 7 ) ( Hu - IL 15 / Hu - IL 15 ) ( SGM 3 / SGM 3 ) ( Mating 2 Progeny ) \times \\ NSG - ( MHC DKO / MHC DKO ( + / W 41 ) Tg ( + / Hu - IL 7 ) ( Hu - IL 15 / Hu - IL 15 ) ( SGM 3 / SGM 3 ) ( Mating 2 Progeny )$$

12.5 of the progeny mice should be homozygous for the MHC DKO, heterozygous for the Kit^{W-41J} mutant (W41) allele, homozygous for the Hu-IL7 transgene, homozygous for the Hu-IL15 transgene, and homozygous for the SGM3 transgenes:

$$NSG - ( MHC DKO / MHC DKO ( + / W 41 ) Tg ( Hu - IL 7 / Hu - IL 7 ) ( Hu - IL 15 / Hu - IL 15 ) ( SGM 3 / SGM 3 ) ( Mating 3 Progeny A )$$

12.5 of the progeny mice should be:

$$NSG - ( MHC DKO / MHC DKO ( W 41 / W 41 ) Tg ( + / Hu - IL 7 ) ( Hu - IL 15 / Hu - IL 15 ) ( SGM 3 / SGM 3 ) ( Mating 3 Progeny B )$$

### Mating 4

$$NSG - ( MHC DKO / MHC DKO ( + / W 41 ) Tg ( Hu - IL 7 / Hu - IL 7 ) ( Hu - IL 15 / Hu - IL 15 ) ( SGM 3 / SGM 3 ) ( Mating 3 Progeny A ) \times \\ NSG - ( MHC DKO / MHC DKO ( W 41 / W 41 ) Tg ( + / Hu - IL 7 ) ( Hu - IL 15 / Hu - IL 15 ) ( SGM 3 / SGM 3 ) ( Mating 3 Progeny B )$$

25% of the progeny mice should be homozygous for the MHC DKO, homozygous for the Kit^{W-41J} mutant (W41) allele, homozygous for the Hu-IL7 transgene, homozygous for the Hu-IL15 transgene, and homozygous for the SGM3 transgenes:

$$NSG - ( MHC DKO / MHC DKO ( W 41 / W 41 ) Tg ( Hu - IL 7 / Hu - IL 7 ) ( Hu - IL 15 / Hu - IL 15 ) ( SGM 3 / SGM 3 ) ( Mating 4 Progeny )$$

### Mating 5

$$NSG - ( MHC DKO / MHC DKO ( W 41 / W 41 ) Tg ( Hu - IL 7 / Hu - IL 7 ) ( Hu - IL 15 / Hu - IL 15 ) ( SGM 3 / SGM 3 ) ( Mating 4 Progeny ) \times NSG \text{} - ( MHC DKO / MHC DKO ( W 41 / W 41 ) Tg ( Hu - IL 7 / Hu - IL 7 ) ( Hu - IL 15 / Hu - IL 15 ) ( SGM 3 / SGM 3 ) ( Mating 4 Progeny )$$

The NSG-(MHC DKO/MHC DKO)(W41/W41) Tg(Hu-IL7/Hu-IL7)(Hu-IL15/Hu-IL15)(SGM3/SGM3) progeny strain will be maintained by sib mating.

### Example 4: Generation of NSG-MHC DKO Tg(Hu-IL7)(Hu-IL15)(SGM3) (Strain 12)

### Mating 1

$$NSG - Tg ( Hu - IL 15 / Hu - IL 15 ) ( SGM 3 / SGM 3 ) ( Strain 8 ) \times NSG - ( + / MHC DKO ) Tg ( + / Hu - IL 7 ) ( Hu - IL 15 / Hu - IL 15 ) Strain 9 Mating 1 Progeny )$$

25% of the progeny mice should be heterozygous for the MHC DKO, hemizygous for the Hu-IL7 transgene, homozygous for the Hu-IL15 transgene, and hemizygous for the SGM3 transgenes:

$$NSG - ( + / MHC DKO ) Tg ( + / Hu - IL 7 ) ( Hu - IL 15 / Hu - IL 15 ) ( + / SGM 3 ) ( Mating 1 Progeny )$$

### Mating 2

$$NSG - ( + / MHC DKO ) {T}_{g} ( + / Hu - IL 7 ) ( Hu - IL 15 / Hu - IL 15 ) ( + / SGM 3 ) ( Mating 1 Progeny ) \times NSG - Tg ( Hu - IL 7 / Hu - IL 7 ) ( Hu - IL 15 / Hu - IL 15 ) ( Strain )$$

12.5% of the progeny mice should be heterozygous for the MHC DKO, homozygous for the Hu-IL7 transgene, homozygous for the Hu-IL15 transgene, and hemizygous for the SGM3 transgenes:

$$NSG - ( + / MHC DKO ) Tg ( Hu - IL 7 / Hu - IL 7 ) ( Hu - IL 15 / Hu - IL 15 ) ( + / SGM 3 ) ( Mating 2 Progeny )$$

### Mating 3

$$NSG - ( + / MHC DKO ) Tg ( Hu - IL 7 / Hu - IL 7 ) ( Hu - IL 15 / Hu - IL 15 ) ( + / SGM 3 ) ( Mating 2 Progeny ) \times NSG - ( + / MHC DKO ) Tg ( Hu - IL 7 / Hu - IL 7 ) ( Hu - IL 15 / Hu - IL 15 ) ( + / SGM 3 ) ( Mating 2 Progeny )$$

12.5% of the progeny mice should be heterozygous for the MHC DKO, homozygous for the Hu-IL7 transgene, homozygous for the Hu-IL15 transgene, and homozygous for the SGM3 transgenes:

$$NSG - ( + / MHC DKO ) Tg ( Hu - IL 7 / Hu - IL 7 ) ( Hu - IL 15 / Hu - IL 15 ) ( SGM 3 / SGM 3 ) ( Mating 3 Progeny )$$

### Mating 4

$$NSG - ( + / MHC DKO ) Tg ( Hu - IL 7 / Hu - IL 7 ) ( Hu - IL 15 / Hu - IL 15 ) ( SGM 3 / SGM 3 ) ( Mating 3 Progeny ) \times NSG - ( + / MHC DKO ) Tg ( Hu - IL 7 / Hu - IL 7 ) ( Hu - IL 15 / Hu - IL 15 ) ( SGM 3 / SGM 3 ) ( Mating 3 Progeny )$$

25% of the progeny mice should be homozygous for the MHC DKO, homozygous for the Hu-IL7 transgene, homozygous for the Hu-IL15 transgene, and homozygous for the SGM3 transgenes:

$$NSG - ( MHC DKO / MHC DKO ) Tg ( Hu - IL 7 / Hu - IL 7 ) ( Hu - IL 15 / Hu - IL 15 ) ( SGM 3 / SGM 3 ) ( Mating 4 Progeny )$$

### Mating 5

$$NSG - ( MHC DKO / MHC DKO ) Tg ( Hu - IL 7 / Hu - IL 7 ) ( Hu - IL 15 / Hu - IL 15 ) ( SGM 3 / SGM 3 ) ( Mating 4 Progeny ) \times NSG - ( MHC DKO / MHC DKO ) Tg ( Hu - IL 7 / Hu - IL 7 ) ( Hu - IL 15 / Hu - IL 15 ) ( SGM 3 / SGM 3 ) ( Mating 4 Progeny )$$

The NSG-(MHC DKO/MHC DKO) Tg(Hu-IL7/Hu-IL7)(Hu-IL15/Hu-IL15)(SGM3/SGM3) progeny strain will be maintained by sib mating.

### Example 5: Generation of NSG-MHC DKO Tg(Hu-IL7)(SGM3) (Strain 13)

### Mating 1

$$NSG - ( + / MHC DKO ) ( + / W 41 ) Tg ( + / Hu - IL 7 ) ( + / Hu - IL 15 ) ( + / SGM 3 ) ( Strain 11 Mating 4 Progeny ) \times NSG - ( SGM 3 / SGM 3 ) ( Strain 14 )$$

3.125% of the progeny mice should be heterozygous for the MHC DKO, hemizygous for the Hu-IL7 transgene, and homozygous for the SGM3 transgenes:

$$NSG - ( + / MHC DKO ) Tg ( + / Hu - IL - 7 ) ( SGM 3 / SGM 3 ) ( Mating 1 Progeny )$$

### Mating 2

$$NSG - ( + / MHC DKO ) Tg ( + / Hu - IL - 7 ) ( SGM 3 / SGM 3 ) ( Mating 1 Progeny ) \times NSG - ( + / MHC DKO ) Tg ( + / Hu - IL - 7 ) ( SGM 3 / SGM 3 ) ( Mating 1 Progeny )$$

6.25% of the progeny mice should be homozygous for the MHC DKO, hemizygous for the Hu-IL7 transgene, and hemizygous for the SGM3 transgenes:

$$NSG - ( MHC DKO / MHC DKO ) Tg ( + / Hu - IL 7 ) ( + / SGM 3 ) ( Mating 2 Progeny A )$$

3.125% of the progeny mice should be heterozygous for the MHC DKO, homozygous for the Hu-IL7 transgene, and homozygous for the SGM3 transgenes:

$$NSG - ( + / MHC DKO ) Tg ( Hu - IL 7 / Hu - IL 7 ) ( SGM 3 / SGM 3 ) ( Mating 2 Progeny B )$$

### Mating 3

$$NSG - ( MHC DKO / MHC DKO ) Tg ( + / Hu - IL 7 ) ( + / SGM 3 ) ( Mating 2 Progeny A ) \times NSG - ( + / MHC DKO ) Tg ( Hu - IL 7 / Hu - IL 7 ) ( SGM 3 / SGM 3 ) ( Mating 2 Progeny B )$$

50% 3.125% of the progeny mice should be homozygous for the MHC DKO, homozygous for the Hu-IL7 transgene, and homozygous for the SGM3 transgenes:

$$NSG - ( MHC / MHC DKO ) Tg ( Hu - IL 7 / Hu - IL 7 ) ( SGM 3 / SGM 3 ) ( Mating 3 Progeny )$$

### Mating 4

$$NSG - ( MHC / MHC DKO ) Tg ( Hu - IL 7 / Hu - IL 7 ) ( SGM 3 / SGM 3 ) ( Mating 3 Progeny ) \times NSG - ( MHC / MHC DKO ) Tg ( Hu - IL 7 / Hu - IL 7 ) ( SGM 3 / SGM 3 ) ( Mating 3 Progeny )$$

The NSG-(MHC DKO/MHC DKO) Tg(Hu-IL7/HuIL7)(SGM3/SGM3) progeny strain will be maintained by sib mating.

### Example 6: Using the NSG-SGM3-W41 Mouse to Investigate the Role of Innate Immunity in FSHD Muscle Pathology

Facioscapulohumeral muscular dystrophy (FSHD) disease progression is associated with muscle inflammation, although its role in FSHD muscle pathology is unknown. Facioscapulohumeral muscular dystrophy is a prevalent epigenetic disease caused by genetic disruptions including contraction in the D4Z4 repeats at the 4qA locus or loss of function mutations in chromatin modifier genes that lead to hypomethylation of the D4Z4 locus and misexpression of the germline transcription factor gene DUX4, the FSHD disease gene. DUX4 is encoded by the terminal D4Z4 repeat and normally functions during early germline development. DUX4 misexpression in muscle disrupts the muscle transcriptome through activation of a large set of germline-specific genes which are surrogate biomarkers of DUX4 expression, although their role in DUX4 muscle pathology is unknown.

DUX4 misexpression alone does not appear to be sufficient to account for FSHD muscle pathology. Although DUX4 misexpression causes muscle toxicity in vitro in patient muscle cells and in vivo in inducible mouse models, the onset of the muscle pathology in FSHD patients is highly variable including early onset and non-manifesting disease. Clinical disease initiates sporadically in facial, scapular, and humeral muscles, which are often affected asymmetrically, and over time involves most muscle groups. Finally, transient expression of DUX4 in transgenic zebrafish and mouse models results in delayed muscle pathology, and transient DUX4 induction in a human myoblast culture model generates H3.X and H3.Y histones marking the DUX4 target genes for delayed muscle toxicity.

Our investigations have focused on the role of innate immunity as a modifier and amplifier of FSHD muscle pathology and disease progression. FSHD disease progression includes immune cell infiltration followed by muscle turnover and fatty and fibrotic replacement, as evidenced by transcriptome and immunohistology assays of patient muscle biopsies and muscles of DUX4 inducible mouse models. DUX4 misexpression inhibits nonsense mediated decay (NMD), leading to the production of abnormally spliced RNAs and aberrant proteins, predicted to elicit Damage Associated Molecular Patterns (DAMPs) known to stimulate an innate immune response. A role for innate immunity in FSHD muscle pathology is also supported by high expression level of complement genes in muscle biopsies and increased levels of complement C3 in blood of FSHD patients.

To investigate the role of innate immunity in FSHD muscle pathology, we developed a novel humanized HSC/muscle engrafted mouse model, NSG-SGM3-W41. This mouse strain has been engineered to selectively expand human innate immune cell lineages following engraftment of umbilical cord blood (UCB) derived hematopoietic stem cells (HSCs) in the absence of irradiation preconditioning, which supports co-engraftment of patient derived FSHD (or unaffected control) muscle stem cells into the mouse tibialis anterior (TA) muscle to produce differentiated FSHD human muscle that expresses the FSHD disease gene, DUX4. Our findings show that FSHD muscle xenografts in HSC engrafted NSG-SGM3-W41 mice preferentially accumulate human macrophages and early B cells, express early complement RNAs encoding activators of both the classical and alternative pathways, and upregulate C3 RNA and protein, the mediator of early complement response. FSHD muscle xenografts also undergo muscle turnover dependent on specific HSC immune donors, supporting the idea that innate immunity directly contributes to FSHD pathology. A role for late complement in muscle turnover is excluded by our results that FSHD muscle xenografts do not express RNAs encoding the complement membrane attack complex (MAC). Based on our findings, we hypothesize that C3 complement activated by the early complement pathway response to FSHD muscle which produces DAMPs and promotes muscle turnover through opsonization of FSHD muscle for macrophage recognition and engulfment.

### Development of the NSG-SGM3-W41 Mouse Model of Human Innate Immunity

To investigate the role of the human innate immune system in FSHD muscle pathology, we developed a mouse strain (NSG-SGM3-W41, Strain 6 above) that supports the co-engraftment and differentiation of human CD34+ hematopoietic stem cells (HSCs) and human muscle stem cells isolated from FSHD and control patient muscle biopsies. The NSG-SGM3-W41 mouse strain was generated first by crossing immune deficient NSG-SGM3 mice that express the human interleukin-3 gene (IL-3), human granulocyte/macrophage-stimulating factor gene (GM-SCF) and human steel factor gene (KITL) with NSG mice expressing CRISPR Cas9 W41J point mutation of the Kit locus to enable efficient multilineage engraftment of HSCs without irradiation.

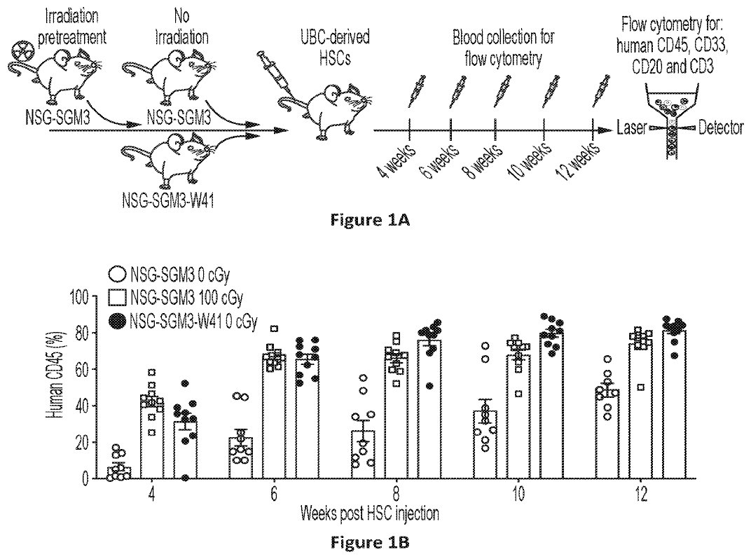

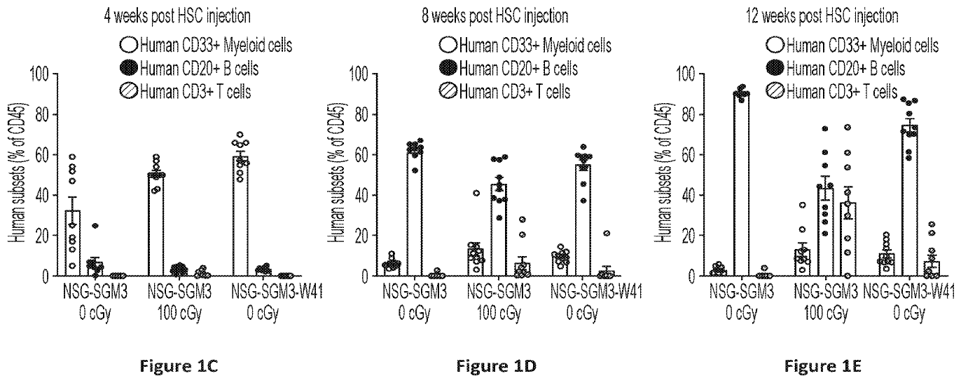

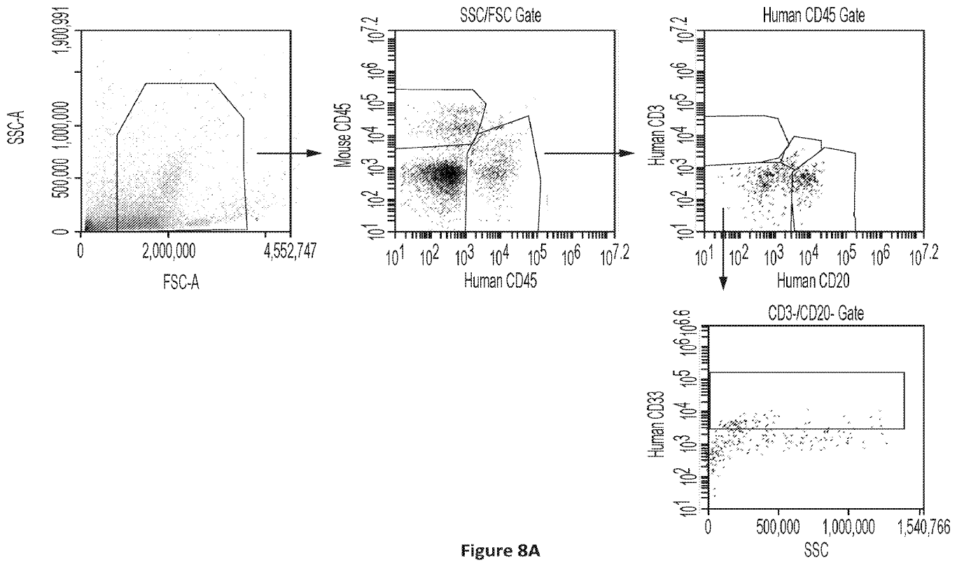

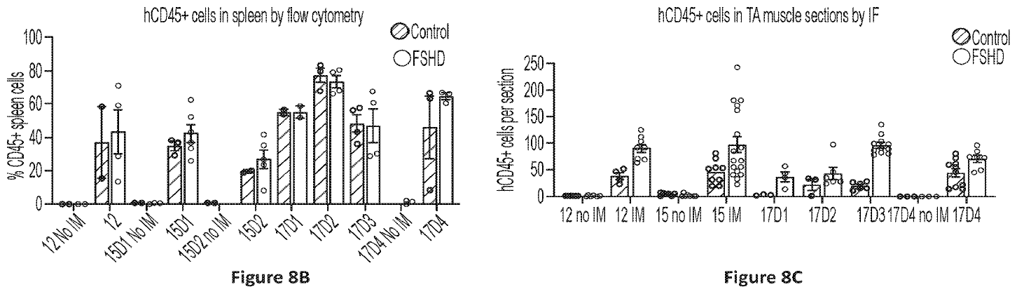

Immune cell development was compared in HSC engrafted NSG-SGM3 mice with or without 100 cGy irradiation pretreatment, and in non-irradiated NSG-SGM3-W41 mice. Mice were injected with 10⁵ CD34+ HSCs isolated from healthy donor UCB, and blood samples from engrafted mice were assayed for circulating human CD45+ immune cells by flow cytometry at 4-, 6-, 8-, 10- and 12-weeks post HSC engraftment (FIGS. 1A, 8A-8C). Results were expressed as the percentages of human CD45+ blood cells (FIG. 1B). Irradiated NSG-SGM3 mice and unirradiated NSG-SGM3-W41 mice both showed 30-40% circulating human CD45+ hematopoietic cells by 4 weeks post HSC injection and this percentage increased to 70-80% at 12 weeks (FIG. 1B). By contrast, the non-irradiated NSG-SGM3 mice injected with HSC had only 6% human CD45+ cells at 4 weeks post HSC engraftment and these cell numbers increased to 48% at 12 weeks (FIG. 1B), with significantly lower engraftment efficiency than the irradiated NSG-SGM3 or non-irradiated NSG-SGM3-W41 mice at all time points.

Specific lineages of human immune cells produced in HSC engrafted NSG-SGM3 mice with or without 100 cGy irradiation pretreatment and in non-irradiated NSG-SGM3-W41 mice were compared in the blood using flow cytometry to determine the percent CD45+ cells that co-expressed CD33, a myeloid cell marker, CD20, a B cell marker, and CD3, a T cell marker. Flow cytometry assays were performed at 4-, 8- and 12-weeks post HSC engraftment (FIGS. 1C-1E). At 4 weeks, mice in all three groups were populated primarily by CD33+ myeloid cells, with unirradiated NSG-SGM3 having the lowest engraftment levels (a mean of 32%) compared to the irradiated NSG-SGM3 mice (50%), and NSG-SGM3-W41 mice (59%) (FIG. 1C), whereas CD20+ B cell numbers were very low and CD3+ T cells were absent. At 8 weeks post HSC injection, all three groups had lower levels of CD33+ myeloid cells compared to 40 and 60% of CD20+ B cells, and several animals in the irradiated NSG-SGM3 group and one in the NSG-SGM3-W41 group had a small number of CD3+ T cells (FIG. 1D). At 12 weeks post HSC injection, the blood of irradiated NSG-SGM3 mice had robust engraftment of CD33+ myeloid cells, CD20+ B cells and CD3+ T cells; non-irradiated NSG-SGM3 mice primarily showed CD20+ B cell engraftment; and NSG-SGM3-W41 mice showed robust engraftment of CD20+ B cells, moderate levels of CD33+ myeloid cells and low levels of CD3⁺ T cells (FIG. 1E). These data show that the NSG-SGM3-W41 mice at 8-weeks post HSC engraftment supported robust development of myeloid and B cells and restricted development of T cells, providing a model for HSC co-engraftment with muscle stem cells to investigate innate immune responses to FSHD muscle.

### Engraftment of FSHD and Control Muscle Stem Cells in HSC Engrafted NSG-SGM3-W41 Mice.

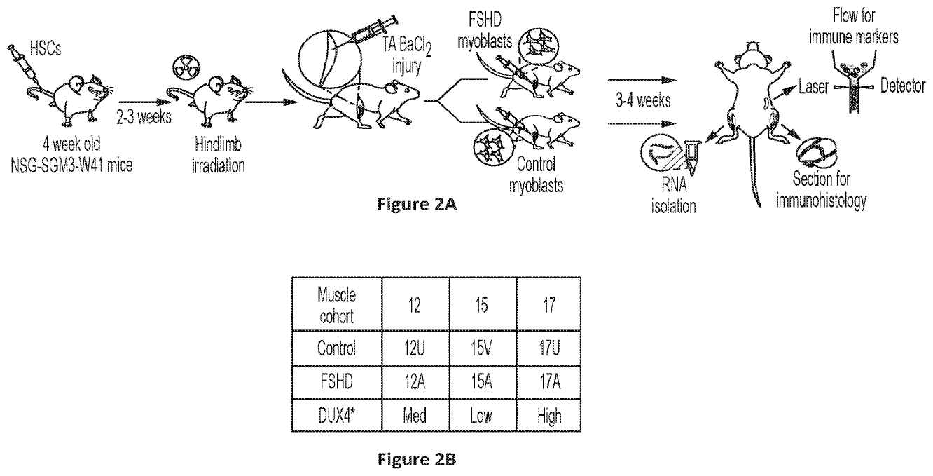

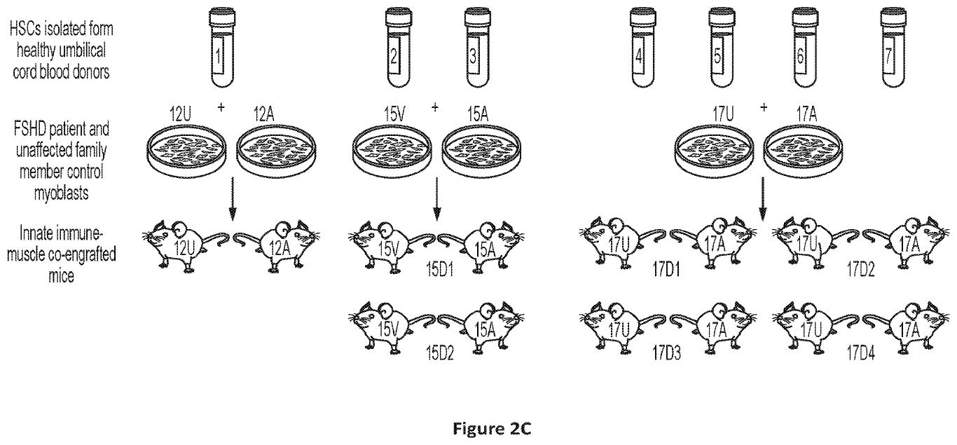

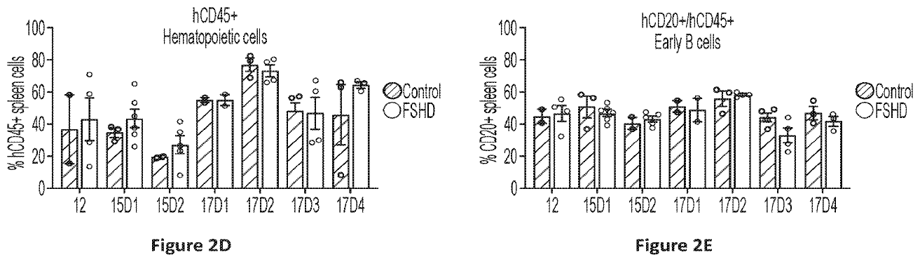

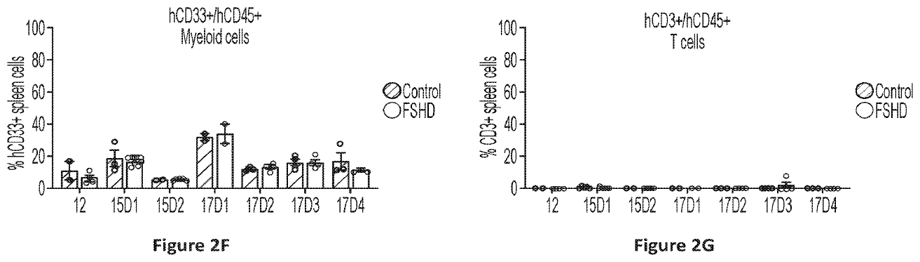

We next investigated whether HSC engrafted NSG-SGM3-W41 mice would support the engraftment and differentiation of patient FSHD and unaffected control muscle stem cells. NSG-SGM3-W41 mice were engrafted with 10⁵ CD34+ HSCs from healthy UCB donors at 4 weeks. Two to three weeks after HSC engraftment, the hindlimbs of the HSC engrafted mice were irradiated to block the growth of host mouse muscle stem cells and Tibialis Anterior (TA) muscles were then injured by barium chloride injection to destroy mouse TA muscle fibers and create a niche for the engraftment and differentiation of human muscle-biopsy derived stem cells (FIG. 2A). Barium chloride injured TA muscles were then engrafted with 10⁶ CD56+ muscle stem cells isolated from muscle biopsies from three FSHD families (12, 15 and 17), including affected FSHD patients (12A, 15A and 17A) and unaffected control first-degree relatives (12U, 15V and 17U) (FIG. 2B). High (17A), medium (12A), and low (15A) DUX4 expressing FSHD cell lines were chosen for experiments to characterize the immune response to FSHD muscle (FIG. 2B). Family 12 muscle stem cells (12A/12U) were co-engrafted into TA muscles of mice with one HSC donor. Family 15 (15A/15V) muscle stem cells were co-engrafted with two different HSC donors (15D1 and 15D2), and family 17 muscle stem cells were engrafted into TA muscles of mice engrafted with HSCs from four different donors (17D1, 17D2, 17D3 and 17D4) (FIG. 2C). Three to four weeks after muscle stem cell engraftment, the mice were euthanized, their spleens were isolated to evaluate the development of human B cells, myeloid cells and T cells, and their TA muscles were processed for immunohistology or RNA expression analyses (FIG. 2A).

HSC immune engraftment was assessed by flow cytometry analysis of spleen cells assayed for CD45+ hematopoietic lineage cells, CD45+/CD20+ B cells, CD45+/CD33+ myeloid cells, and CD45+/CD3+ T cells. Mice engrafted with all combinations of HSC donors and muscle stem cell donors produced comparable levels of hematopoietic CD45+ cells (FIG. 2D), B cells (FIG. 2E), and myeloid cells (FIG. 2F). A representative flow cytometry gating strategy is shown in FIG. 8A. None of the HSC engrafted mice produced CD45+/CD3+T cells, confirming that NSG-SGM3-W41 mice are permissive for the expansion of innate immune cell lineages but not T cell lineages during an 8-week period following HSC engraftment (FIG. 2G). A minority of mice did not engraft with HSCs based on undetectable CD45+ staining by flow cytometry of spleen cells (FIG. 8B) and by immunohistology of TA muscles (FIG. 8C).

### Enhanced Accumulation of CD45+ Innate Immune Cells in FSHD Xenograft Muscle Compared to Control Muscle

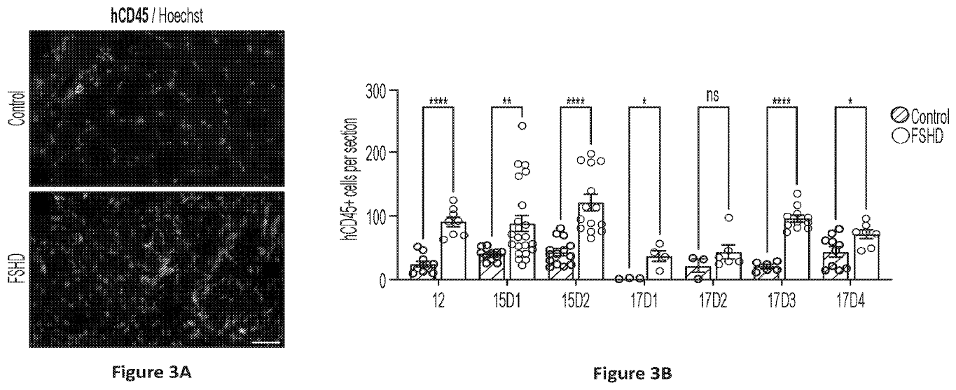

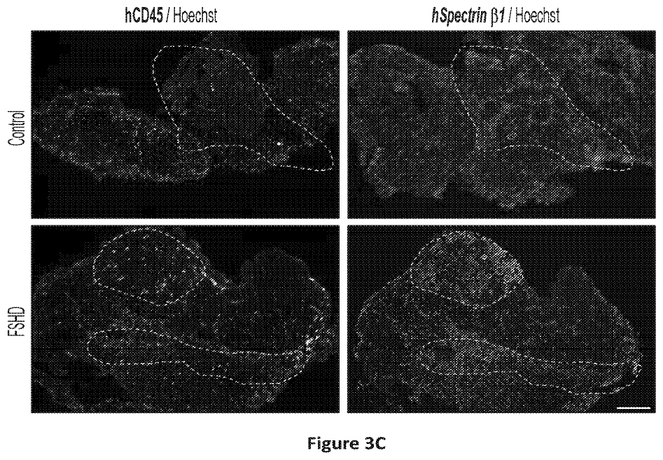

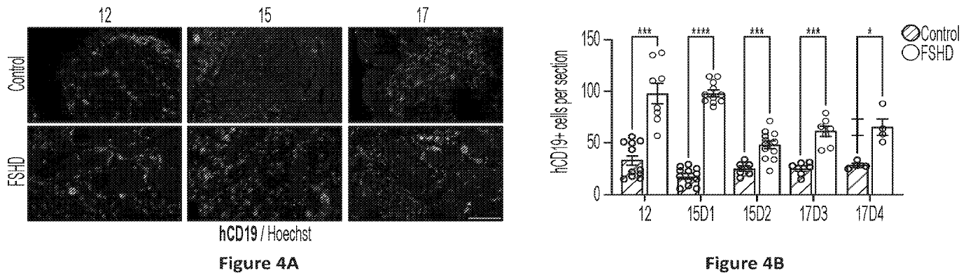

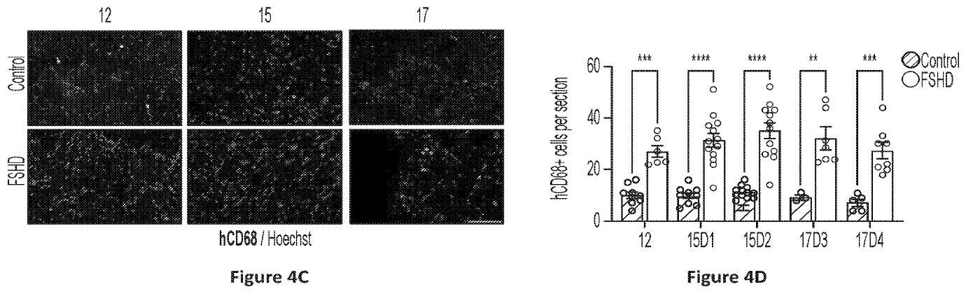

To investigate whether human innate immune cells preferentially infiltrate FSHD muscle xenografts, TA muscles were immunostained for human specific CD45 to identify HSC derived innate immune cells and Hoechst to identify all nuclei (FIG. 3A). While CD45+ immune cells were identified in both FSHD and control TA xenografts (FIG. 3A), CD45+ cells were significantly more abundant in FSHD than control muscle xenografts in 6 of the 7 immune donors (FIG. 3B), reflecting either increased infiltration and/or expansion of human immune cells in FSHD xenografts. To determine whether the human CD45+ cells localized to engrafted human muscle, serial sections of TA muscles were immunostained for human CD45 to show human leukocytes or human spectrin R 1 to show human muscle fibers. CD45+ cells colocalized with spectrin β1+ muscle fibers in FSHD xenografts compared to control muscle (FIG. 4C), providing evidence that immune cells are trophic to FSHD muscle.

Human CD19+ B Cells and CD68+ Macrophages are More Abundant in FSHD than Control Muscle Xenografts

To characterize immune cell types in muscle xenografts, FSHD and control TA muscles were sectioned and immunostained with human CD19 (hCD19) (FIG. 4A), a B cell marker, and human CD68 (hCD68) (FIG. 4C), a macrophage marker. Significantly higher numbers of human B cells and macrophages were present in FSHD compared to control muscle in all immune donors analyzed (FIGS. 4B and D). 17D1 and 17D2 TA muscles were processed for IHC and these tissue samples were not suitable for immunofluorescence assays. These data show that FSHD muscle promotes an influx and/or expansion of macrophages and B cells.

### Muscle Turnover in FSHD Xenografts is Immune Donor Dependent

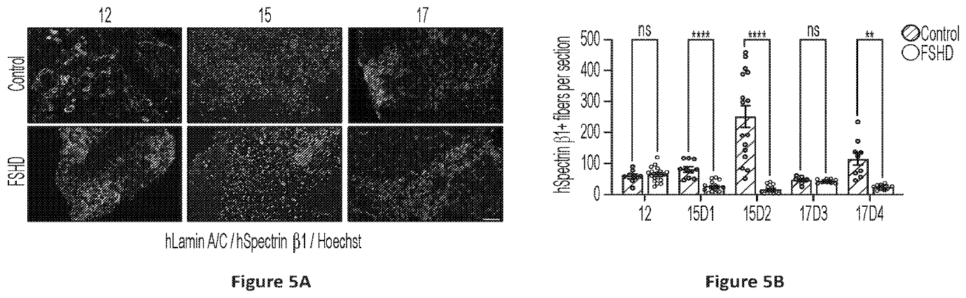

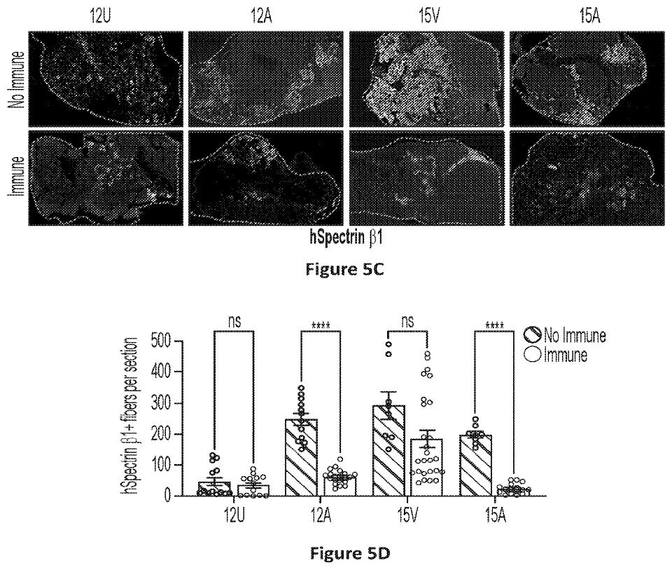

To investigate whether human innate immune cells promote FSHD muscle turnover, TA muscle sections of FSHD and control xenografts were co-immunostained with human-specific antibodies to lamin A/C to identify human nuclei and spectrin β1 to identify differentiated human muscle fibers in immune donors 12, 15D1, 15D2, 17D3 and 17D4. Xenografts from cohorts 17D1 and 17D2 were processed for IHC and were not assayed for immunofluorescence. Lamin A/C+ nuclei and spectrin β1+ muscle fibers were detected in both FSHD and control TA muscle xenografts (FIG. 5A). Notably, FSHD xenografts for cohorts 15D1, 15D2 and 17D4 had significantly fewer spectrin β1+ muscle fibers than control xenografts (FIG. 5B), indicating that FSHD muscle is turning over, whereas FSHD and control xenografts from cohorts 12 and 17D3 maintained similar levels of spectrin β1+ muscle fibers, although these FSHD muscle were infiltrated with immune cells (FIGS. 3 and 4). To confirm that innate immune cell co-engraftment reduced proliferative capacity, the numbers of spectrin R 1+ muscle fibers were compared in immune engrafted mice and mice that failed to develop immune systems in cohorts 12 and 15 (FIGS. 5C-5D). Significantly fewer spectrin β1+ muscle fibers were observed in immune engrafted 12A and 15A muscle xenografts, while similar numbers of fibers were observed in 12U and 15V with or without immune engraftment, showing that control muscle does not undergo turnover in response to their co-engrafted immune donor (FIG. 5D).

### Inflammatory Response to FSHD Muscle is Immune Donor Dependent

To analyze the immune and muscle gene expression profiles of FSHD and control xenografts, we designed a custom NanoString RNA expression quantification panel containing human specific probes to immune, muscle and DUX4 target genes. This NanoString panel assayed the expression of 204 inflammation genes, 3 muscle genes (MYH8, MYL2, and MEF2C) and 2 DUX4 transcriptional target genes (LEUTX and MBD3L2). NanoString assays were performed on FSHD and control xenografts from 3 different FSHD families (12, 15 and 17) in NSG-SGM3-W41 mice engrafted with 7 different HSC immune donors, as described above (FIG. 2C).

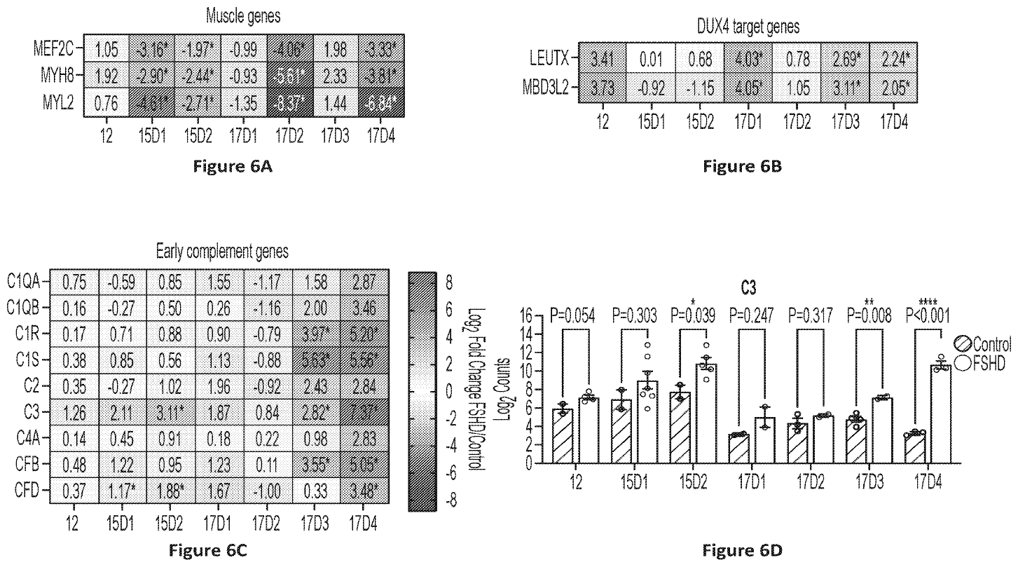

To identify differentially expressed genes in FSHD vs control xenografts, NanoString counts for each mouse were log₂ transformed and averaged within each muscle donor and immune donor combination before calculating the fold change of FSHD vs control expression for all genes within all 7 of the HSC donors. Expression levels of muscle genes were significantly reduced by up to 200-fold (log₂ fold change −8.37) in 4 of the 7 HSC donor groups (15D1, 15D2, 17D2 and 17D4) (FIG. 6A), providing evidence for the differential turnover of FSHD muscle in these cohorts as also observed in immunohistological assays of spectrin β1+ muscle fibers in FSHD xenografts from cohorts 15D1, 15D2 and 17D4 (FIG. 5). By contrast, cohorts 12 and 17D3 had increased expression of muscle genes in FSHD vs control, suggestive of a regenerative response of FSHD muscle in these xenografts (FIG. 6A). Differentially expressed human immune genes included early complement pathway genes in both the classical and alternative pathways, including C3, which is the key mediator of the complement response; C1R, C1S, C1QA, and C1QB, which comprise the C1 complex of the classical complement pathway that initiates complement activation through interactions with pathogens or DAMPs; the C2 serine proteinase; and CFB and CFD, which are unique to the alternative pathway and also responsive to DAMPs (FIG. 6). Their levels of expression varied based on muscle cohort and HSC immune donor. 17D2 FSHD xenografts had low expression of complement genes and high muscle turnover, but also accumulated macrophages and B cell, suggesting that 17D2 donor immune cells efficiently targeted FSHD muscle for turnover (FIG. 4). Notably, C3 expression trended higher in FSHD xenograft muscles of the three FSHD cohorts responding to all seven immune donors. Expression of all human late complement RNAs including C5-C9, which encode for components of the Membrane Attack Complex (MAC), were undetectable in both FSHD and control muscles. Furthermore, NSG-SGM3-W41 mice are deficient in the C5 complement component and therefore cannot elicit a mouse host MAC response. In addition to complement genes, several chemokines including CXCL1, CXCL2, CXCL6, CXCL9, CXCL10 and CCL13 were expressed higher in FSHD xenografts in several cohorts compared to controls.

### Muscle Fibers in FSHD Xenografts Show Increased Deposits of C3

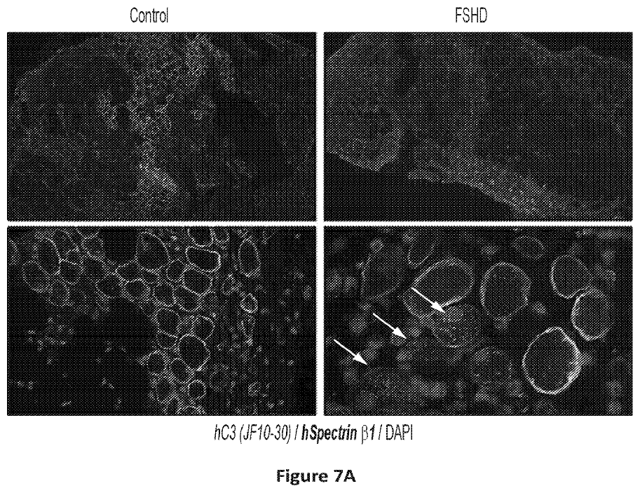

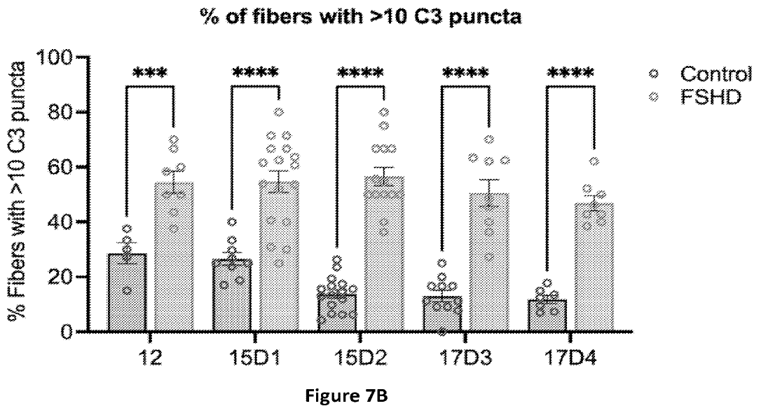

Human immune-muscle engrafted TA muscle cryosections were immunostained with human specific antibodies to spectrin β1 to identify human muscle fibers and human specific C3 to investigate the localization of human C3 in relation to humanized muscle regions. Immunohistology assays of cohorts 12, 15D1, 15D2, 17D3 and 17D4 showed abundant expression and localization of C3 in FSHD muscle fibers compared to control fibers (FIG. 7A), supporting the NanoString RNA expression findings. Human C3 was detected only in humanized regions of engrafted mouse TA muscles and localized on the surface and within FSHD muscle fibers but was also concentrated in areas surrounding muscle fibers, which we postulate are enriched in human immune cells. We quantified the abundance of C3 puncta inside spectrin β1+ human muscle fibers and found that in all cohorts analyzed, FSHD engrafted sections had significantly higher percentage of fibers containing greater than 10 C3 puncta (FIG. 7B). The high numbers of puncta are highlighted by the white arrows in the high magnification FSHD image (FIG. 7A). Mouse C3 was not detected in xenografts by immunohistology, using a mouse specific C3 antibody (data not shown). Taken together, these data demonstrate that FSHD and control xenografts express human C3 and FSHD xenografts have a larger percentage of muscle fibers that are highly decorated with C3.

### Discussion and Conclusions

We have developed a humanized innate immune/muscle mouse model to investigate the role of innate immune responses to FSHD muscle. Our findings establish that FSHD muscle xenografts from all three FSHD families generate innate inflammatory responses to respective immune donors. FSHD xenografts elicited enhanced infiltration of macrophages and early B cells compared to control xenografts, showing that FSHD muscle is trophic for and/or promotes the expansion of innate immune cells within FSHD xenografts. FSHD xenografts expressed human early complement RNAs and human C3 RNA and protein as part of an innate immune inflammatory response to FSHD muscle, and the expression of mouse C3 protein could not be detected. FSHD xenografts also underwent what we hypothesize is immune donor-dependent muscle turnover, as shown by the differential muscle turnover responses of 17A FSHD xenografts to four different immune donors (FIG. 6). Two of the four 17A immune donors, 17D2 and 17D4 promoted extensive muscle turnover compared to donor 17D1, which promoted lower turnover, and donor 17D3 promoted increased muscle gene expression, perhaps reflecting a regenerative response to muscle damage by the resident muscle stem cells in xenografts, as observed in FSHD muscle. Although muscle turnover in 17D3 was not observed, macrophages and early B cells from both 17D3 and 17D4 HSC donors preferentially accumulated in FSHD muscle xenografts and expressed elevated C3, showing robust inflammatory responses. HSC donor dependent differences in FSHD muscle turnover may reflect quantitative differences in immune donor potency relative to the fixed end point of our assay. Studies are ongoing to establish live animal imaging reporter muscle stem cell lines to establish muscle xenografts. This will enable monitoring of the kinetics of muscle turnover of individual muscle xenografts during stem cell differentiation and maturation in response to different immune donors. Our data demonstrates that individual immune donors produce innate immune cells that have different capacities for immune responses, perhaps modeling aspects of the observed variability in disease progression in FSHD patients and families with multiple affected members. Innate immune responses vary in populations, so it is expected that we observe variability in the immune responses from our healthy immune donors in response to FSHD xenografts. Future studies of immune donor variability in this model will address these possibilities.

Our innate immune muscle xenograft model establishes a role for innate immunity in FSHD muscle pathology, but the mechanisms by which FSHD muscle is trophic for innate immune cells remain to be investigated. Our working hypothesis is that FSHD muscle attracts macrophages and early B cells through the DUX4-mediated production of DAMPs to stimulate production of complement factor C3 and early complement classical and alternative pathway convertases for processing C3 to C3b. By this mechanism, C3b would bind to and opsonize FSHD muscle for recognition and turnover by macrophage engulfment. Current studies are focused on investigations of the functions of DUX4 and C3 in FSHD muscle turnover using DUX4 siRNA therapeutics and early complement pathway-specific immunotherapeutics, with the goal of developing combinatorial therapeutics to treat FSHD disease initiation and progression.

We have generated a humanized innate immune-FSHD muscle xenograft model using NSG-SGM3-W41 mice to investigate the innate immune response to FSHD muscle. In standardizing our model using muscle biopsy-derived myoblasts from three FSHD patients and paired healthy controls, and HSCs from seven healthy donors, we found that in all cohorts, human B cells and macrophages preferentially infiltrate FSHD muscle. While immune cells infiltrated FSHD xenograft from all cohorts, FSHD muscle turnover was only observed in four of the seven cohorts suggesting that the response is immune donor dependent. Finally, we observed higher expression of complement genes from both the classic and alternative pathways in FSHD engrafted muscles than to control engrafted muscles, suggesting a potential mechanism and novel druggable pathway to ameliorate FSHD muscle pathology.

### Methods

Mouse Model Generation NOD.Cg-Kit^{em1Mvw} Prkdc^{scid} IL2rg^{tm1Wjl} Tg(CMV-IL3,CSF2,KITL)Eav/MloySzJ (NSG-SGM3-W41) mice were developed as described above in Table 1. NSG-SGM3. The W41 mutation in the mouse Kit gene, consisting of a G to A point mutation in the kinase domain (V831M), was made directly in NSG zygotes using CRISPR-Cas9 and oligo-mediated homology directed repair, as described previously. To reduce the potential for off-target mutations, a truncated guide was used to target the sequence: GCACGACTGCCCGTGAAG (SEQ ID NO: 1) and the NSG-Kit^{W41} allele was generated using the donor oligonucleotide template: AGGGGAGGTGGCTGGAGGTCACAAGGTTTAAGGTCCTCGTCTATCGCTGTCTTCATT AGCTGCTTGAATTTGCTGTGTTCCGTTCTAGGCACGACTGCCCATGAAGTGGATGGC ACCAGAGAGCATTTTCAGCTGCGTGTACACATTTGAAAGTGATGTCTGGTCCTATGG GATTTTCCTCTGGGAGCTCTTCTCCTTAG (SEQ ID NO: 2). NSG-SGM3 mice were intercrossed with NSG-Kit^{W41} mice and further crosses were made to fix all genes to homozygosity in NSG-SGM3 Kit^{W41} mice.
Isolation of Human Umbilical Cord Blood (UCB)-HSC and Engraftment into Mice

Human UCB was obtained in accordance with the Committee for the Protection of Human Subjects in Research guidelines of the University of Massachusetts Chan Medical School. UCB was provided by the University of Massachusetts Memorial Umbilical Cord Blood Donation Program. Groups of 4- to 8-week-old male and female NSG-SGM3-W41 mice were injected IV with CD3-depleted (Miltenyi Biotech) human UCB containing 1×105 CD34+ HSCs. At the indicated time points, flow cytometry analyses of the blood of engrafted mice quantified the engraftment of the human immune system. For experimental studies, mice with >10% peripheral human CD45+ cells and >5% human CD3+ T cells were used.

### Flow Cytometry

For analysis of human immune system development in HSC engrafted NSG-SGM3-W41 mice, the following monoclonal antibodies specific for human antigens were used: human CD45 (2D1), CD3 (UCHT1), CD20 (2H7) and CD33 (WM53). Anti-mouse CD45 (30F-11) was used to exclude mouse leukocytes. The antibodies were purchased from BD Biosciences, Inc. (CA) or BioLegend (CA). Single-cell suspensions of the spleens were prepared from engrafted mice, and whole blood was collected in heparin. Single cell suspensions of 5×10⁵ splenic cells in 50 μl or 100 μl of whole blood was washed with FACS buffer (PBS supplemented with 2% fetal bovine serum, (HyClone, UT) and 0.02% sodium azide (Sigma, MO) and then pre-incubated with rat anti-mouse FcR11b (clone 2.4G2, BD Biosciences, CA) to block Fc binding. Specific antibodies against cell surface antigens were then added to the samples and incubated for 30 min at 4° C. Stained samples were then washed and fixed with 2% paraformaldehyde for cell suspensions or treated with BD FACS lysing solution for whole blood. At least 100,000 events were acquired on LSRII instrument (BD Biosciences, CA) or Aurora (Cytek Biosciences, CA). Data analysis was performed with FlowJo software (Tree Star, Inc., OR).

### Cell Culture

CD56+ FAC sorted FSHD and control myoblasts from families 12, 15 and 17 were cultured on 0.1% gelatin (sigma G9391) coated 15 cm dishes in HMP medium (Ham's F10 (Cellgro 10-070-CV) supplemented with 20% FBS (Hyclone SH30071.03), and 1% chick embryo extract (made in house)) and passaged using TrypLE (ThermoFisher) when 70% confluence was reached.

### Muscle Xenografts

NSG-SGM3-W41 mice were used in accordance with the Institutional Animal Care and Use Committee (IACUC) at the UMass Chan Medical School. Mice were anesthetized with ketamine/xylazine and their hindlimbs were subjected to 18 Gy of irradiation using a Faxitron CellRad X-ray cabinet (Faxitron Bioptics LLC) to ablate the host mouse satellite cell population. Lead shields were used to limit radiation exposure to hindlimbs only. One day after irradiation, mice were anesthetized using an isoflurane vaporizer (SurgiVet model 100) and Tibialis Anterior (TA) muscles were injected with 50 μl of 1.2% Barium Chloride (Sigma) bilaterally to degenerate mouse muscle. Three days after muscle injury, 1×10⁶ CD56+ biopsy-derived myoblasts were resuspended in 50 μl 1 mg/mL laminin (Sigma, L2020) in phosphate buffered saline (PBS) and injected bilaterally into the body of TA muscles. Xenoengrafted mice were euthanized 3-4 weeks post engraftment by C02 asphyxiation followed by cervical dislocation. For immunohistology experiments, TA muscles were embedded in Tissue-Tek O.C.T compound (Sakura) and frozen on liquid nitrogen cooled isopentane and kept at −80° C. until cryosectioning. For RNA isolation, xenoengrafted TA muscles were snap frozen in liquid nitrogen and kept at −80° C. until RNA isolation.

RNA isolation for NanoString

RNA was isolated from xenoengrafted TA muscles using Aurum Total RNA Fatty and Fibrous Tissue kit (Bio-Rad) per the manufacturer's specifications. For NanoString digital RNA quantification, 150 ng of total RNA was used for each xenografted TA muscle. A custom inflammation NanoString panel with human-specific probes for muscle protein genes (MEF2C, MYH8 and MYL2), DUX4 target genes (LEUTX and MBD3L2), inflammation genes and multiple housekeeping genes was used for all analysis on an nCounter Sprint profiler (NanoString Technologies, Seattle, WA). Raw mRNA counts for each TA sample were normalized to a panel of housekeeping genes (RPL13A, GAPDH, GUSB, HRPT1, PGK1, TUBB, and VCP) using nSolver software (NanoString Technologies, Seattle, WA).

### TA Sectioning and Immunohistology

Frozen TA muscles embedded in Tissue-Tek OCT compound (Sakura) were cryosectioned using a Leica CM3050 S Cryostat. Tissue sections 10 μm thick were mounted onto Superfrost Plus glass microscope slides (Fisher Scientific) and kept at −20° C. When thawed, the sections were fixed with ice cold acetone for 10 minutes at −20° C. For lamin A/C (mab636) and spectrin β1 (NCL-SPEC1) co-staining, we employed the “mouse-on-mouse” (M.O.M) kit (Vector Laboratories) to reduce non-specific antibody staining per the manufacturer's specifications. Antibodies were used sequentially then slides were incubated with Hoechst block for 10 minutes. For human CD45 (Dako, M0701), CD19 (Abcam, ab134114), CD68 (Agilent, clone PG-M1) or human specific C3 (ThermoFisher, JF10-30) immunostaining, primary antibodies were incubated with slides overnight at 4° C. followed by 2×5 min PBS washes. The corresponding secondary antibodies were added and incubated at room temperature for one hour followed by 2×5 min PBS washes. Slides were incubated with Hoechst for 10 mins at room temperature then dried and coverslips were mounted with Fluorogel. Fluorescent images were taken using a Leica DMR fluorescence microscope with IKona monochrome high sensitivity 6MP camera with Sony sensor.

### Statistics

NanoString, flow cytometry and immunostaining quantification data are shown as the mean±SEM. Statistical differences for NanoString RNA expression data and immunofluorescence quantification were evaluated using Welch's t test and were considered significant when the P value was less than 0.05 (*=P<0.05, **=P<0.01, ***=P<0.001, ****=P<0.0001). Statistical analyses were performed using Prism V9 (Graphpad Software LLC).

### Example 7—Engraftment of Unfractionated Human Umbilical Cord Blood (UCB) into NSG Mouse Strains

8-12 week old NSG-MHC DKO Tg(Hu-IL15), NSG-MHC DKO, and NSG-Tg(Hu-IL7)(Hu-IL15) mice (Strains 7, 2, and 5, respectively, in Table 1 above) were irradiated with 200 cGy radiation and then engrafted with unfractionated human umbilical cord blood (UCB) containing 5×10⁴ CD34⁺ human hematopoietic stem cells (HSCs) and 1.9×10⁶ human CD3⁺ T-cells. 2/10, 7/8, and 0/10 of the NSG-MHC DKO Tg(Hu-IL15), NSG-MHC DKO, and NSG-Tg(Hu-IL7)(Hu-IL15) mice, respectively, survive to at least 6 weeks after irradiation and engraftment. These survival numbers suggest that engraftment of unfractionated human UCB in NSG mice expressing Hu-IL15 decreases overall survival. To increase survival to at least 6 weeks, the amount of irradiation can be decreased, the number human T cells engrafted can be decreased, human interleukin-4 can be expressed (e.g., using any expression vector provided herein), or some combination thereof can be performed.

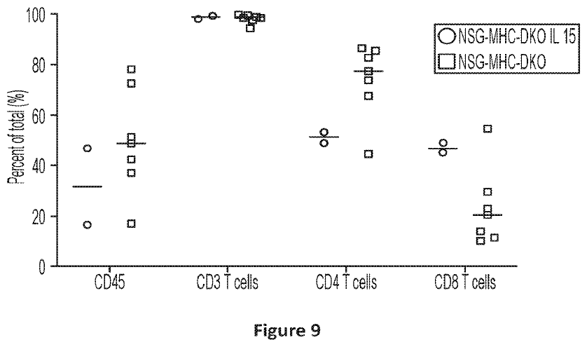

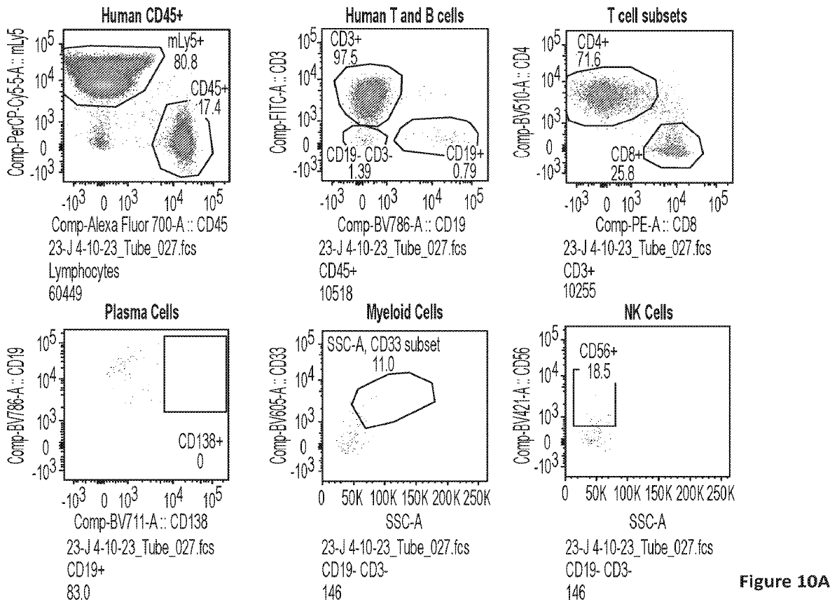

The mean percentage of human immune cells being expressed in the surviving NSG-MHC DKO Tg(Hu-IL15) and NSG-MHC DKO mice was assessed 6 weeks after irradiation and engraftment (FIG. 9). The mean percentage of human CD45⁺ monocyte cells was ˜30% and ˜50% of total CD45+ cells in NSG-MHC DKO Tg(Hu-IL15) and NSG-MHC DKO mice, respectively. The mean percentage of human CD3⁺ T cells was ˜100% of total CD3⁺ T cells in NSG-MHC DKO Tg(Hu-IL15) and NSG-MHC DKO mice. The mean percentage of CD4⁺ T cells was ˜55% and ˜80% of total CD4⁺ T cells in NSG-MHC DKO Tg(Hu-IL15) and NSG-MHC DKO mice. The mean percentage of human CD8⁺ T cells was ˜45% and ˜20% of total CD8⁺ T cells in NSG-MHC DKO Tg(Hu-IL15) and NSG-MHC DKO mice.

These results demonstrate that transgenic expression of human IL15 in NSG-MHC DKO mice decreases human leukocyte cell (CD45⁺) and CD4⁺ T cell proliferation relative to NSG-MHC DKO mice. These results further demonstrate that transgenic expression of human IL15 in NSG-MHC DKO mice increases CD8⁺ T cell proliferation relative to NSG-MHC DKO mice. Taken together, these results suggest that engraftment of unfractionated UCB as described above in mice expressing human IL15 decreases the expression of human CD45⁺ monocyte cells and favors the expression of CD8⁺ T cells at the expense of CD4⁺ T cells.

### Example 8—Engraftment of Unfractionated Human UCB into NSG Mouse Strains with Decreased Irradiation and T-Cell Engraftment

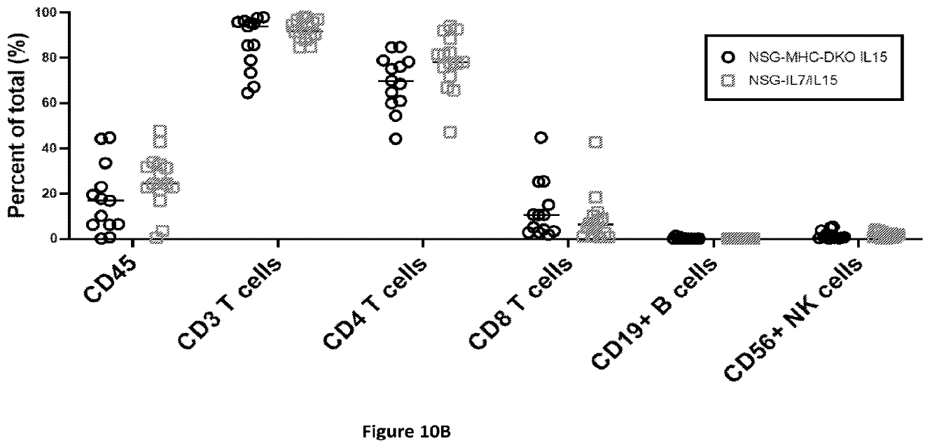

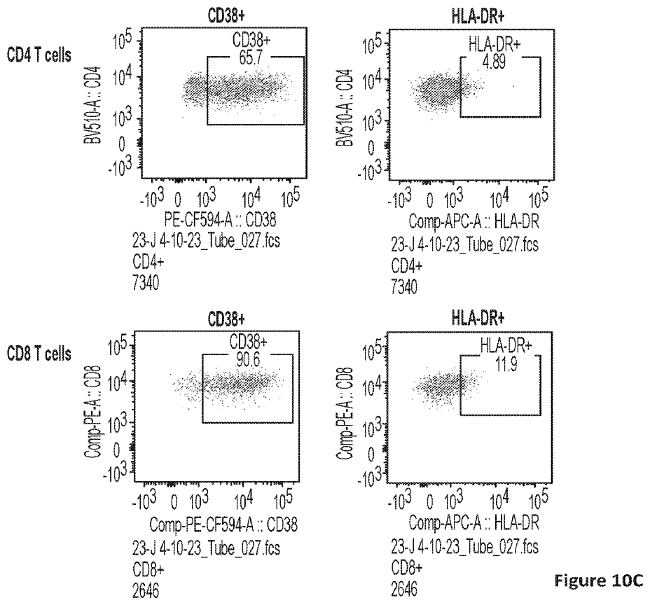

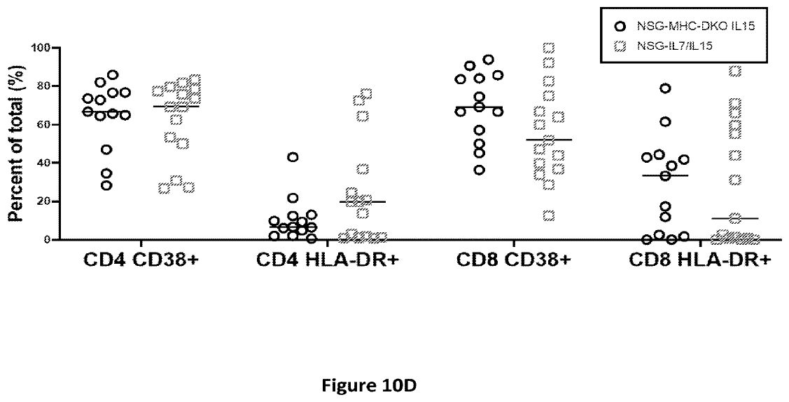

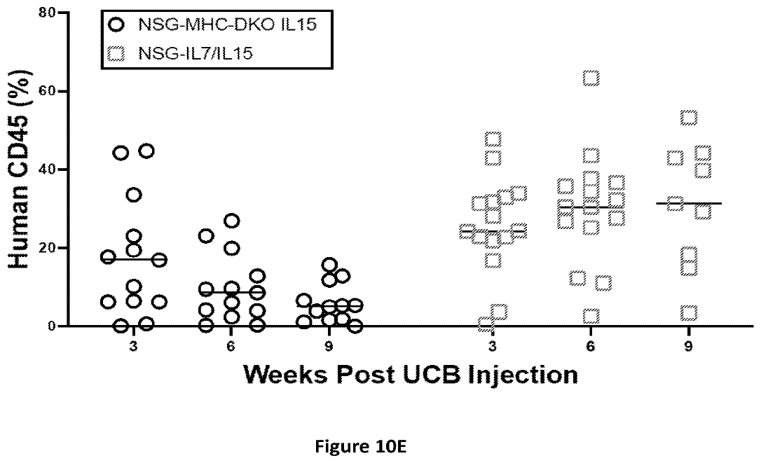

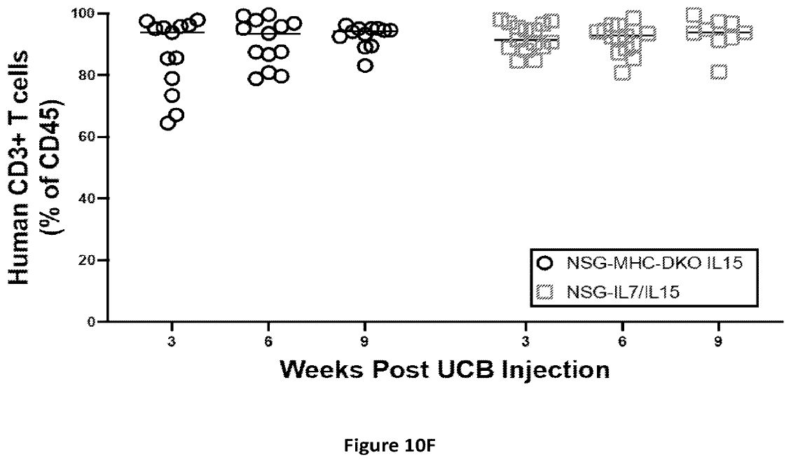

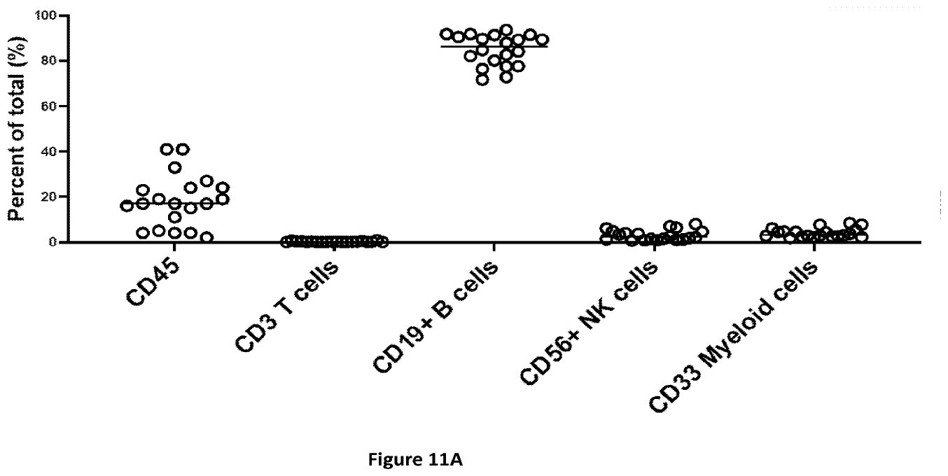

8-12 week old NSG-MHC DKO Tg(Hu-IL15) and NSG-Tg(Hu-IL7)(Hu-IL15) mice (Strains 7 and 5, respectively, in Table 1 above) were irradiated with 100 cGy radiation and then engrafted with unfractionated human umbilical cord blood (UCB) containing 5×10⁴ CD34⁺ human hematopoietic stem cells (HSCs) and 1.0×10⁶ human CD3⁺ T-cells. An AAV-IL4 vector was also introduced into the mice to express IL4. The amount of radiation and the number of human CD3+ T cells engrafted in the unfractionated UCB were roughly halved compared with the experiment in Example 7. 13/13 and 14/14 NSG-MHC DKO Tg(Hu-IL15) and NSG-Tg(Hu-IL7)(Hu-IL15) mice survived to 3 weeks after irradiation and engraftment (FIG. 10B), and 12/13 and 9/14 NSG-MHC DKO Tg(Hu-IL15) and NSG-Tg(Hu-IL7)(Hu-IL15) mice survived to 3 weeks after irradiation and engraftment (FIG. 10E). These results indicate that decreased radiation, decreased human CD3+ T cell engraftment, and/or expression of AAV-IL4 increase the survival of NSG mice expressing human-IL15.

The percentage of human immune cells being expressed in the NSG-MHC DKO Tg(Hu-IL15) and NSG-Tg(Hu-IL7)(Hu-IL15) mice was assessed at 3 weeks after irradiation and engraftment (FIGS. 10A, 10B). The mean percentage of human CD45⁺ leukocytes was ˜18% and ˜25% in NSG-MHC DKO Tg(Hu-IL15) and NSG-Tg(Hu-IL7)(Hu-IL15) mice, respectively. The mean percentage of human CD3⁺ T cells was ˜97% of total CD3⁺ T cells in NSG-MHC DKO Tg(Hu-IL15) and NSG-MHC DKO mice. The mean percentage of human CD19⁺ B cells was ˜1% of total CD45⁺ cells in NSG-MHC DKO Tg(Hu-IL15) and NSG-Tg(Hu-IL7)(Hu-IL15) mice, respectively. The mean percentage of human CD4⁺ T cells was ˜72% and ˜80% of total CD4⁺ T cells in NSG-MHC DKO Tg(Hu-IL15) and NSG-Tg(Hu-IL7)(Hu-IL15) mice, respectively. The mean percentage of human CD8⁺ T cells was ˜15% and ˜10% of total CD8⁺ T cells in NSG-MHC DKO Tg(Hu-IL15) and NSG-Tg(Hu-IL7)(Hu-IL15) mice, respectively. The mean expression of human CD56⁺ natural killer cells was ˜1% of total CD45⁺ cells in NSG-MHC DKO Tg(Hu-IL15) and NSG-Tg(Hu-IL7)(Hu-IL15) mice. Taken together, these results suggest that expression of MHC DKO alleles and/or expression of Hu-IL7 alters human CD45⁺ leukocyte expression, human CD4⁺ T cell expression, and human CD8⁺ T cell expression relative in NSG mice engrafted with unfractionated UCB as described above.

The mean percentage of numerous human T cell populations being expressed in the NSG-MHC DKO Tg(Hu-IL15) and NSG-Tg(Hu-IL7)(Hu-IL15) mice was also assessed at 3 weeks after irradiation and engraftment (FIGS. 10C, 10D). The mean percentage of human CD4+CD38⁺ T cells was ˜65% of total CD4+CD38⁺ T cells in NSG-MHC DKO Tg(Hu-IL15) and NSG-Tg(Hu-IL7)(Hu-IL15) mice, respectively. The mean percentage of human CD4⁺ HLA-DR⁺ T cells was ˜5% and ˜20% of total CD4⁺ HLA-DR⁺ T cells, respectively. The mean percentage of human CD8⁺ CD38⁺ T cells was ˜70% and ˜50% of total CD8⁺ CD38⁺ T cells, respectively. The mean percentage of human CD8⁺ HLA-DR⁺ T cells was ˜35% and ˜10% of total CD8⁺ HLA-DR⁺ T cells, respectively. Taken together, these results suggest that expression of MHC DKO alleles and/or expression of Hu-IL7 alters human CD4⁺ HLA-DR⁺ T cell expression, CD8⁺ CD38⁺ T cell expression, and CD8⁺ HLA-DR⁺ T cell expression in NSG mice engrafted with unfractionated UCB as described above.

The percentage of human CD45⁺ cells in total CD45⁺ cells being expressed in the NSG-MHC DKO Tg(Hu-IL15) and NSG-Tg(Hu-IL7)(Hu-IL15) mice was also assessed at 3, 6, and 9 weeks after irradiation and engraftment (FIG. 10E). The mean percentage of human CD45⁺ cells decreases from ˜18% to ˜10% to ˜8% in the NSG-MHC DKO Tg(Hu-IL15) mice at 3, 6, and 9 weeks after irradiation and engraftment. The mean percentage of human CD45⁺ cells is ˜25%, ˜30%, and ˜32% in the NSG-Tg(Hu-IL7)(Hu-IL15) mice at 3, 6, and 9 weeks after irradiation and engraftment. Of these human CD45⁺ cells, ˜92% were CD3⁺ T cells in the NSG-MHC DKO Tg(Hu-IL15) mice and NSG-Tg(Hu-IL7)(Hu-IL15) mice (FIG. 10F). Taken together, these results suggest that expression of MHC DKO alleles decreases the percentage of human CD45⁺ cells from 3 weeks to 9 weeks in NSG mice engrafted with unfractionated UCB as described above relative to NSG mice that do not express MHC DKO alleles and do express Hu-IL7.

### Example 9—Split Cell Injections of Unfractionated UCB into NSG Mouse Strains

NSG-MHC DKO Tg(Hu-IL15) mice (Strain 7) were treated with split cell injections for human engraftment. In these split cell injections, CD34+ hematopoietic stem cells (HSCs) are selected (e.g., isolated) from human UCB and engrafted (injected) into NSG-MHC DKO Tg(Hu-IL15) mice. The flow-through from this selection, which contains at least T cells, B cells, macrophages, dendritic cells, and natural killer (NK) cells is referred to as the negative fraction. The negative fraction is cryo-preserved and engrafted (injected) into the NSG-MHC DKO Tg(Hu-IL15) mice 6 weeks after these mice are engrafted with CD34⁺ HSCs.

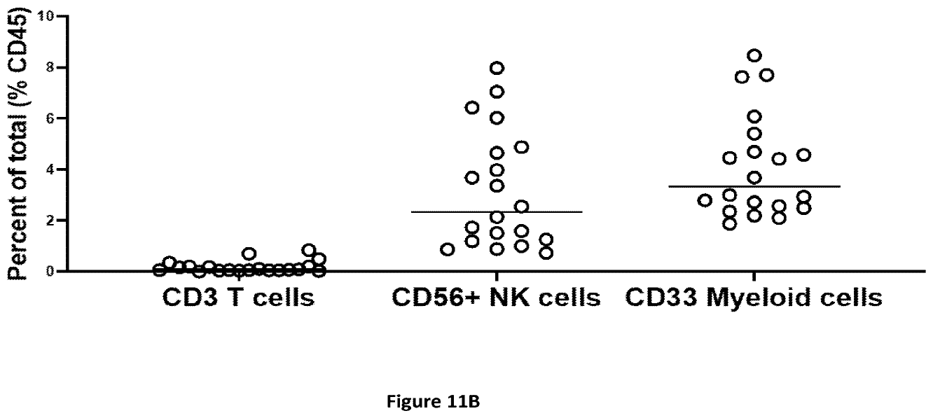

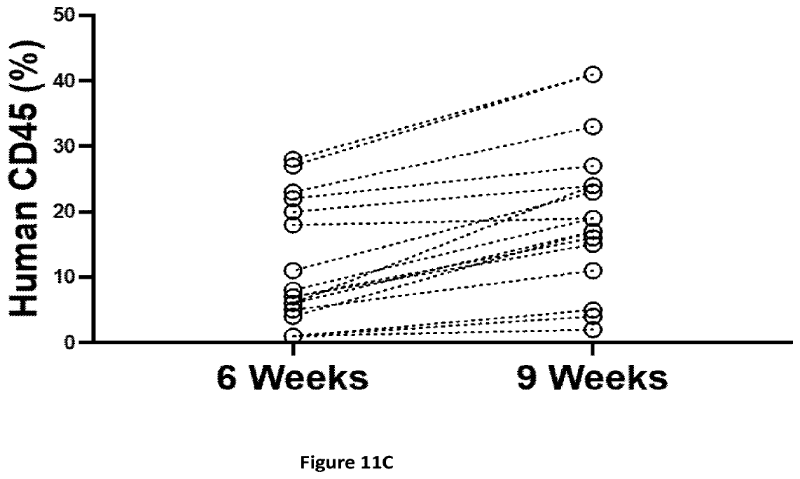

The mean percentage of human immune cells being expressed in the NSG-MHC DKO Tg(Hu-IL15) mice was assessed at 6 and 9 weeks after human UCB split cell engraftment (FIGS. 11A-11C). The mean percentage of human CD45+ leukocytes was ˜18% of total in NSG-MHC DKO Tg(Hu-IL15). This percentage of human CD45⁺ cells was increased from ˜1%-25% to ˜2%-40% from 6 weeks to 9 weeks after split cell engraftment in the NSG-MHC DKO Tg(Hu-IL15) mice (FIG. 11C). The mean percentage of human CD3⁺ T cells was ˜1% of total CD3⁺ T cells in NSG-MHC DKO Tg(Hu-IL15) mice. The mean percentage of human CD19⁺ B cells was ˜90% of total CD45⁺ cells in NSG-MHC DKO Tg(Hu-IL15) mice. The mean percentage of human CD56+NK cells was ˜2% of total CD56⁺ cells in NSG-MHC DKO Tg(Hu-IL15) mice. The mean percentage of human CD33+ myeloid cells was ˜3% of total CD33⁺ cells in NSG-MHC DKO Tg(Hu-IL15) mice (FIGS. 11A, 11B). Taken together, these results suggest that the percentage of human CD3⁺ T cells decreases with split cell engraftment as described above (comparing FIG. 10F above to FIGS. 11A and 11B). Additionally, human CD45⁺ cell expression increases from 6 weeks to 9 weeks.

All references, patents and patent applications disclosed herein are incorporated by reference with respect to the subject matter for which each is cited, which in some cases may encompass the entirety of the document.

The indefinite articles “a” and “an,” as used herein the specification and in the claims, unless clearly indicated to the contrary, should be understood to mean “at least one.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The terms “about” and “substantially” preceding a numerical value mean±10% of the recited numerical value.

Where a range of values is provided, each value between the upper and lower ends of the range are specifically contemplated and described herein.

What is claimed is:

1. An immunodeficient non-obese diabetic (NOD) mouse comprising:
(a) an endogenous Il2rg allele comprising a null mutation, an endogenous Prkdc allele comprising a null mutation, and an endogenous Kit allele comprising a null mutation; and
(b) a transgene encoding human interleukin 3 (huIL3), a transgene human granulocyte-macrophage colony-stimulating factor (huGM-CSF), and a transgene human Steel factor (huSCF).

2. The immunodeficient mouse of claim 1, wherein the immunodeficient mouse has been engrafted with human hematopoietic stem cells.

3. The immunodeficient mouse of claim 1, wherein the immunodeficient mouse has been engrafted with human peripheral blood mononuclear cells.

4. The immunodeficient mouse of any one of claims 1-3, wherein the immunodeficient mouse has been engrafted with diseased human cells.

5. The immunodeficient mouse of claim 4, wherein the diseased human cells are obtained from a subject having a genetic disorder.

6. The immunodeficient mouse of claim 5, wherein the genetic disorder is facioscapulohumeral muscular dystrophy (FSHD).

7. The immunodeficient mouse of claim 6, wherein the diseased human cells are human muscle cells.

8. The immunodeficient mouse of claim 7, wherein the human muscle cells are CD56+ muscle stem cells.

9. A method comprising:
administering human hematopoietic stem cells to a non-irradiated immunodeficient mouse of claim 1, wherein the human HSCs develop into innate immune cells; and
administering human diseased cells to the non-irradiated immunodeficient mouse.

10. The method of claim 9, wherein about 10⁴ to about 10⁶ human HSCs are administered.

11. The method of claim 9 or 10, wherein the human diseased cells are administered about 4 to about 10 weeks post administration of the human HSCs.

12. The method of any one of claims 9-11, wherein about 10⁴ to about 10⁶ human diseased cells, optionally muscle cells, further optionally CD56+ muscle stem cells, are administered.

13. The method of claim 12, wherein the human diseased cells are obtained from a subject having facioscapulohumeral muscular dystrophy (FSHD).

14. The method of any one of claims 9-13, further comprising administering a therapeutic modality to the immunodeficient mouse.

15. The method of any one of claims 9-14 further comprising assaying for a response of the innate immune cells to the human diseased cells.

16. The method of claim 15, wherein the response is an inflammatory response.

17. An immunodeficient non-obese diabetic (NOD) mouse comprising:
(a) an endogenous Il2rg allele comprising a null mutation, an endogenous Prkdc allele comprising a null mutation, an endogenous H2-K allele comprising a null mutation (H2-K^{null}); an endogenous H2-D allele comprising a null mutation (H2-D^{null}); an endogenous H2-A allele comprising a null mutation (H2-A^{null}); and
(b) a transgene encoding human interleukin 15 (huIL15).

18. An immunodeficient non-obese diabetic (NOD) mouse comprising:
(a) an endogenous Il2rg allele comprising a null mutation and an endogenous Prkdc allele comprising a null mutation; and
(b) a transgene encoding human interleukin 15 (huIL15) and a transgene encoding human interleukin 7 (huIL7).

19. A method comprising:
administering human hematopoietic stem cells (HSCs) or human peripheral blood mononuclear cells (PBMCs) to an immunodeficient mouse of claim 17 or 18, wherein the human HSCs develop into innate immune cells, optionally wherein the mouse is non-irradiated; and
administering human diseased cells to the immunodeficient mouse.

20. The method of claim 19, wherein about 10⁴ to about 10⁶ human HSCs or human PBMCs are administered.

21. The method of claim 19 or 20, wherein the human diseased cells are administered about 4 to about 10 weeks post administration of the human HSCs or human PBMCs.

22. The method of any one of claims 19-21, wherein about 10⁴ to about 10⁶ human diseased cells are administered.

23. The method of any one of claims 19-22, further comprising administering a therapeutic modality to the immunodeficient mouse.

24. The method of any one of claims 19-23 further comprising assaying for a response of the innate immune cells to the human diseased cells.

25. The method of claim 24, wherein the response is an inflammatory response.


SEQUENCE LISTING (2 sequences)

SEQ ID NO: 1 - DNA, 18 bp, synthetic construct
  source 1..18: mol_type = other DNA
gcacgactgcccgtgaag

SEQ ID NO: 2 - DNA, 200 bp, synthetic construct
  source 1..200: mol_type = other DNA
aggggaggtggctggaggtcacaaggtttaaggtcctcgtctatcgctgtcttcattagctgcttgaatttgctgtgttccgttctaggcacgactgcccatgaagtggatggcaccagagagcattttcagctgcgtgtacacatttgaaagtgatgtctggtcctatgggattttcctctgggagctcttctccttag